(19) United States
(12) Patent Application Publication
(10) Pub. No.: US 2025/0018040 A1
(43) Pub. Date: Jan. 16, 2025

(54) ANTI-MESOTHELIN POLYPEPTIDES, PROTEINS, AND CHIMERIC ANTIGEN RECEPTORS

(71) Applicant: The United States of America, as represented by the Secretary, Department of Health and Human Servic, Bethesda, MD (US)

(72) Inventors: Ira H. Pastan, Chevy Chase, MD (US); Masanori Onda, Germantown, MD (US); Mitchell Ho, Urbana, MD (US); Xiu-fen Liu, Clarksville, MD (US); Tapan Bera, Frederick, MD (US); Anirban Chakraborty, Bethesda, MD (US)

(73) Assignee: The United States of America, as represented by the Secretary, Department of Health and Human Servic, Bethesda, MD (US)

(21) Appl. No.: 18/710,726

(22) PCT Filed: Dec. 16, 2022
(86) PCT No.: PCT/US2022/081766

§ 371 Date: May 16, 2024

Related U.S. Application Data
(60) Provisional application No. 63/290,761, filed on Dec. 17, 2021.

(51) Int. Cl. A61K 39/00; A61P 35/00; C07K 16/32

(52) U.S. Cl. CPC A61K 39/464468; A61K 39/4611; A61K 39/4631; A61P 35/00; C07K 16/32; C07K 2317/24; C07K 2317/31

(57) ABSTRACT

Polypeptides, proteins, and chimeric antigen receptors (CARs) that specifically bind to human mesothelin₅₈₂₋₅₉₈ (IP-NGYLVLDLSMQEALS) (SEQ ID NO: 1) are disclosed. Anti-mesothelin binding moieties, nucleic acids, recombinant expression vectors, host cells, populations of cells, pharmaceutical compositions, and conjugates relating to the poly peptides, proteins, and CARS are disclosed. Methods of reducing mesothelin shed from cell membranes, methods of detecting the presence of cancer, and methods of treating or preventing cancer are also disclosed.

### CROSS-REFERENCE TO RELATED APPLICATION

This patent application claims the benefit of U.S. Provisional Patent Application No. 63/290,761, filed Dec. 17, 2021, which is incorporated by reference in its entirety herein.

### STATEMENT REGARDING FEDERALLY SPONSORED RESEARCH OR DEVELOPMENT

This invention was made with Government support under project number IZIABC008753-35 by the National Institutes of Health, National Cancer Institute. The Government has certain rights in the invention.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 140,871 Byte XML file named “764980_ST25.XML,” dated Dec. 16, 2022.

### BACKGROUND OF THE INVENTION

Cancer is a public health concern. Despite advances in treatments such as chemotherapy, the prognosis for many cancers may be poor. Accordingly, there exists an unmet need for additional treatments for cancer.

### BRIEF SUMMARY OF THE INVENTION

An aspect of the invention provides a polypeptide which specifically binds to human mesothelin₅₈₂₋₅₉₈ (IPNGYLVLDLSMQEALS) (SEQ ID NO: 1) and which comprises:
(A) the light chain complementary determining region (VL CDR) 1 amino acid sequence of SEQ ID NO: 17;
the VL CDR2 amino acid sequence of SEQ ID NO: 19;
the VL CDR3 amino acid sequence of SEQ ID NO: 21;
the heavy chain complementary determining region (VH CDR) 1 amino acid sequence of SEQ ID NO: 24;
the VH CDR2 amino acid sequence of SEQ ID NO: 26; and
the VH CDR3 amino acid sequence of SEQ ID NO: 28;
(B) the VL CDR1 amino acid sequence of SEQ ID NO: 17;
the VL CDR2 amino acid sequence of SEQ ID NO: 19;
the VL CDR3 amino acid sequence of SEQ ID NO: 21;
the VH CDR1 amino acid sequence of SEQ ID NO: 31;
the VH CDR2 amino acid sequence of SEQ ID NO: 33; and
the VH CDR3 amino acid sequence of SEQ ID NO: 35;
(C) the VL CDR1 amino acid sequence of SEQ ID NO: 38;
the VL CDR2 amino acid sequence of SEQ ID NO: 40;
the VL CDR3 amino acid sequence of SEQ ID NO: 42;
the VH CDR1 amino acid sequence of SEQ ID NO: 24;
the VH CDR2 amino acid sequence of SEQ ID NO: 26; and
the VH CDR3 amino acid sequence of SEQ ID NO: 28; or
(D) the VL CDR1 amino acid sequence of SEQ ID NO: 38;
the VL CDR2 amino acid sequence of SEQ ID NO: 40;
the VL CDR3 amino acid sequence of SEQ ID NO: 42;
the VH CDR1 amino acid sequence of SEQ ID NO: 31;
the VH CDR2 amino acid sequence of SEQ ID NO: 33; and
the VH CDR3 amino acid sequence of SEQ ID NO: 35.

An aspect of the invention provides a protein which specifically binds to human mesothelin₅₈₂₋₅₉₈ (IPNGYLVLDLSMQEALS) (SEQ ID NO: 1) and which comprises:
(A) a first polypeptide chain comprising the light chain complementary determining region (VL CDR) 1 amino acid sequence of SEQ ID NO: 17; the VL CDR2 amino acid sequence of SEQ ID NO: 19; and the VL CDR3 amino acid sequence of SEQ ID NO: 21; and
a second polypeptide chain comprising the heavy chain complementary determining region (VH CDR) 1 amino acid sequence of SEQ ID NO: 24; the VH CDR2 amino acid sequence of SEQ ID NO: 26; and the VH CDR3 amino acid sequence of SEQ ID NO: 28;
(B) a first polypeptide chain comprising the VL CDR1 amino acid sequence of SEQ ID NO: 17; the VL CDR2 amino acid sequence of SEQ ID NO: 19; and the VL CDR3 amino acid sequence of SEQ ID NO: 21; and
a second polypeptide chain comprising the VH CDR1 amino acid sequence of SEQ ID NO: 31; the VH CDR2 amino acid sequence of SEQ ID NO: 33; and the VH CDR3 amino acid sequence of SEQ ID NO: 35;
(C) a first polypeptide chain comprising the VL CDR1 amino acid sequence of SEQ ID NO: 38; the VL CDR2 amino acid sequence of SEQ ID NO: 40; and the VL CDR3 amino acid sequence of SEQ ID NO: 42; and
a second polypeptide chain comprising the VH CDR1 amino acid sequence of SEQ ID NO: 24; the VH CDR2 amino acid sequence of SEQ ID NO: 26; and the VH CDR3 amino acid sequence of SEQ ID NO: 28; or

(D) a first polypeptide chain comprising the VL CDR1 amino acid sequence of SEQ ID NO: 38; the VL CDR2 amino acid sequence of SEQ ID NO: 40; and the VL CDR3 amino acid sequence of SEQ ID NO: 42; and
a second polypeptide chain comprising the VH CDR1 amino acid sequence of SEQ ID NO: 31; the VH CDR2 amino acid sequence of SEQ ID NO: 33; and the VH CDR3 amino acid sequence of SEQ ID NO: 35.

Another aspect of the invention provides a chimeric antigen receptor (CAR) comprising an antigen binding domain, a transmembrane (TM) domain, and an intracellular T cell signaling domain, wherein the antigen binding domain has antigen specificity for human mesothelin₅₈₂₋₅₉₈ (IPNGYLVLDLSMQEALS) (SEQ ID NO: 1), and wherein the antigen binding domain comprises:
(A) the light chain complementary determining region (VL CDR) 1 amino acid sequence of SEQ ID NO: 17; the VL CDR2 amino acid sequence of SEQ ID NO: 19; the VL CDR3 amino acid sequence of SEQ ID NO: 21; the heavy chain complementary determining region (VH CDR) 1 amino acid sequence of SEQ ID NO: 24; the VH CDR2 amino acid sequence of SEQ ID NO: 26; and the VH CDR3 amino acid sequence of SEQ ID NO: 28;
(B) the VL CDR1 amino acid sequence of SEQ ID NO: 17; the VL CDR2 amino acid sequence of SEQ ID NO: 19; the VL CDR3 amino acid sequence of SEQ ID NO: 21; the VH CDR1 amino acid sequence of SEQ ID NO: 31; the VH CDR2 amino acid sequence of SEQ ID NO: 33; and the VH CDR3 amino acid sequence of SEQ ID NO: 35;
(C) the VL CDR1 amino acid sequence of SEQ ID NO: 38; the VL CDR2 amino acid sequence of SEQ ID NO: 40; the VL CDR3 amino acid sequence of SEQ ID NO: 42; the VH CDR1 amino acid sequence of SEQ ID NO: 24; the VH CDR2 amino acid sequence of SEQ ID NO: 26; and the VH CDR3 amino acid sequence of SEQ ID NO: 28;
(D) the VL CDR1 amino acid sequence of SEQ ID NO: 38; the VL CDR2 amino acid sequence of SEQ ID NO: 40; the VL CDR3 amino acid sequence of SEQ ID NO: 42; the VH CDR1 amino acid sequence of SEQ ID NO: 31; the VH CDR2 amino acid sequence of SEQ ID NO: 33; and the VH CDR3 amino acid sequence of SEQ ID NO: 35; or
(E) the VL CDR1 amino acid sequence of SEQ ID NO: 3; the VL CDR2 amino acid sequence of SEQ ID NO: 5; the VL CDR3 amino acid sequence of SEQ ID NO: 7; the VH CDR1 amino acid sequence of SEQ ID NO: 10; the VH CDR2 amino acid sequence of SEQ ID NO: 12; and the VH CDR3 amino acid sequence of SEQ ID NO: 14.

Another aspect of the invention provides a bispecific, biparatopic CAR comprising a first antigen binding domain comprising any of the inventive polypeptides. proteins, or anti-mesothelin binding moieties described herein with respect to other aspects of the invention, and a second antigen binding domain having antigen specificity for a human mesothelin epitope other than human mesothelin₅₈₂₋₅₉₈ (IPNGYLVLDLSMQEALS) (SEQ ID NO: 1).

Another aspect of the invention provides a nucleic acid comprising a nucleotide sequence encoding a CAR construct comprising: (a) a first CAR, wherein the first CAR is any of the inventive CARs described herein: (b) a second CAR comprising a second antigen binding domain, a second TM domain, and a second intracellular T cell signaling domain: and (c) a cleavage sequence: wherein the cleavage sequence is positioned between the first and second CARs, and wherein the second CAR specifically binds to a human mesothelin epitope other than human mesothelin₅₈₂₋₅₉₈ (IPNGYLVLDLSMQEALS) (SEQ ID NO: 1).

Another aspect of the invention provides a nucleic acid comprising a nucleotide sequence encoding: (a) any of the inventive polypeptides described herein: (b) a CAR comprising an antigen binding domain, a TM domain, and an intracellular T cell signaling domain: and (c) a cleavage sequence: wherein the cleavage sequence is positioned between the polypeptide and the CAR, and wherein the CAR specifically binds to a human mesothelin epitope other than human mesothelin₅₈₂₋₅₉₈ (IPNGYLVLDLSMQEALS) (SEQ ID NO: 1).

Further aspects of the invention provide related anti-mesothelin binding moieties, nucleic acids, recombinant expression vectors, host cells, populations of cells, conjugates, and pharmaceutical compositions relating to the polypeptides, proteins, and CARs of the aspects of the invention.

Another aspect of the invention provides a method of reducing mesothelin shed from cell membranes, the method comprising administering to the cells any of the inventive polypeptides, proteins, anti-mesothelin binding moieties, conjugates, CARs, nucleic acids, recombinant expression vectors, host cells, populations of cells, or pharmaceutical compositions described herein, in an amount effective to reduce mesothelin shed from the cell membranes of the cells.

Still another aspect of the invention provides a method of detecting the presence of cancer in a mammal, the method comprising: (a) contacting a sample comprising one or more cells from the mammal with any of the inventive polypeptides, proteins, anti-mesothelin binding moieties, conjugates, CARs, nucleic acids, recombinant expression vectors, host cells, populations of cells, or pharmaceutical compositions described herein, thereby forming a complex, and (b) detecting the complex, wherein detection of the complex is indicative of the presence of cancer.

Still another aspect of the invention provides a method of treating or preventing cancer in a mammal, the method comprising administering to the mammal any of the inventive polypeptides, proteins, anti-mesothelin binding moieties, conjugates, CARs, nucleic acids, recombinant expression vectors, host cells, populations of cells, or pharmaceutical compositions described herein, in an amount effective to treat or prevent cancer in the mammal.

Another aspect of the invention provides a method of treating or preventing cancer in a mammal, the method comprising administering to the mammal: (a) any of the inventive polypeptides, proteins, anti-mesothelin binding moieties, conjugates, CARs, nucleic acids, recombinant expression vectors, host cells, populations of cells, or pharmaceutical compositions described herein, and (b) a further agent that specifically binds to a human mesothelin epitope other than human mesothelin₅₈₂₋₅₉₈ (IPNGYLVLDLSMQEALS) (SEQ ID NO: 1) and inhibits the growth of mesothelin-expressing cells.

### BRIEF DESCRIPTION OF THE SEVERAL VIEWS OF THE DRAWING(S)

FIG. 1 shows an image of gel showing the results of a Western blot assay testing the ability of mAb 15B6 or mAb MN to bind to full length Fc-mesothelin (Fc-MSLN) or shed mesothelin (Shed MSLN).

FIG. 2A shows images of the results of an immunohistochemistry assay testing the ability of mAb 15B6 to bind to mesothelin-expressing tumor samples of mesothelioma, esophagus, lung, and PDAC.

FIGS. 2B-2C show the results of flow cytometry studies that were carried out to test the ability of mAb 15B6 to bind to mesothelin-expressing tumor cell lines KLM1 (2B), OVCAR8 (2B), RH29 (2B), RH16 (2C), CT26-M (2C), T3M4 (2C), and KB31 (2C) and to mesothelin-negative control tumor cell line KLM1_E10 (2B).

FIGS. 3A-3B are graphs showing the concentration of mesothelin (MLN) (ng/mL) detected in the medium following incubation of mAb 15B6 or control mAb MPC11with cancer cell line OVCAR8, KLM1, T3M4, RH16, or RH29 (3A) or cancer cell line A431/M18 or CT26-M (3B).

FIGS. 3CA-3E are graphs showing the concentration of mesothelin (MLN) (ng/mL) detected in the medium following incubation of various concentrations of mAb 15B6 with cancer cell line OVCAR8 (3C), CT26-M (3D), or A431/M18 (3E).

FIGS. 4A-4D are graphs showing the percentage of target cells killed following co-culture with effector cells at the indicated effector to target (E:T) ratios. Effector cells were 15B6 CAR-T cells or SS1 CAR-T cells. CAR-T cells with mock transfection was used as a control. Target cells were cancer cell line OVCAR8 (4A), RH29 (4B), KLM1 (4C), or A431 (4D).

FIGS. 4E-4G are graphs showing the concentration (pg/mL) of TNF-alpha (E), IFN-gamma (F), and IL-2 (G) secreted following co-culture of the effector cells described for

FIGS. 4A-4D with target cell OVCAR8, RH29, KLM1, or control (mesothelin-negative) cancer cell line KLM1E10.

FIGS. 5A-5B are graphs showing the percentage of OVCAR8 cells killed following co-culture with 15B6 CAR-T cells or SS1 CAR-T cells in the presence of the indicated concentrations of truncated (5A) or full-length (5B) mesothelin (μg/mL).

FIG. 5C is a graph showing the percentage of KLM1 cells killed following co-culture with 15B6 CAR-T cells or SS1 CAR-T cells in the presence (+) or absence (−) of ascites from mesothelioma patient (RH16).

FIGS. 6A-6B are graphs showing tumor growth as measured by bioluminescent imaging (radiance: photons/second (bioluminescence generated by the reaction of luciferase transfected into OVCAR-8 cells, which were injected into mice)) over a time period of 35 days. Mice were treated with 1×10⁷ (i) SS1 CAR-T cells, (ii) 15B6 CAR-T cells, or (iii) control CAR T cells or were left untreated. Higher total flux indicates greater tumor burden. Squares indicate non-targeting control CAR-T cells. 6A presents the results from imaging the mice from the back (dorsal). 6B presents the results from imaging the mice from the front (ventral).

FIGS. 7A-7B are graphs showing the percentage of target cells killed following co-culture with effector cells at the indicated effector to target (E:T) ratios. Effector cells were 15B6 CAR-T cells or CAR-T cells prepared with the Fv of Construct No. 9 (L1H1), Construct No. 10 (L1H2), Construct No. 11 (L2H1), or Construct No. 12 (L2H2). Target cells were cancer cell line OVCAR8 (7A) or RH29 (7B).

FIGS. 8A-8C are schematics illustrating the general structures of CARs according to aspects of the invention: CD8HTM CAR (8A), CD28HTM CAR (8B), and IgG4H/CD28TM CAR (8C).

FIGS. 9A-9B are schematics illustrating the general structures of bicistronic vectors according to aspects of the invention: a bicistronic vector encoding a first CAR with the Fv of mAb 15B6 and a second CAR with the Fy of mAb YP218 (9A) and a bicistronic vector encoding a CAR with the Fy of mAb YP218 and the Fy of mAb 15B6 (9B).

FIGS. 10-11 are graphs showing tumor volume measured at various time points (day's post-tumor transplantation) in tumor-bearing mice treated with saline, control cells, or 15B6 CAR-T cells. Data re plotted up to about Day 100 for mice treated with 15B6 CAR-T cells and up to about Day 52 for mice treated with control cells (FIG. 11), up to about Day 65 for mice treated with control cells (FIG. 10), up to about Day 57 for mice treated with PBS (FIG. 11), or up to about Day 81 for mice treated with PBS (FIG. 10).

FIGS. 12A-12F are graphs showing the tumor size measured at various time points (days post-tumor transplantation) in each one of six individual tumor-bearing mice (A-F, respectively) treated in FIG. 11. The arrows indicate the day that treatment was administered.

FIGS. 13A-13D are schematics illustrating the structures of BiTEs according to aspects of the invention: BiTE 1: 15B6 scFv and anti-CD3 scFv in tandem (A), BiTE 4: Diabody format including 15B6 scFv and anti-CD3 scFv in tandem and linked to hulgG1 Fc (B), BITE 5: 15B6 Fv and anti-CD3 Fv knobs-into holes (KiH) (D), and BiTE 6: 15B6 scFv, ALB1, and anti-CD3 scFv in tandem (C). ALB1 is a single domain antibody against human serum albumin (C).

FIGS. 13E and 13F are schematics illustrating the structures of BiTEs comprising anti-mesothelin humanized SS1 (huSS1) Fv or anti-CD19 Fv: BiTE 7 (E): huSS1 Fv and anti-CD3 Fv KiH. BiTE 8 (F): anti-CD19 Fv and anti-CD3 Fv KiH.

FIGS. 14A-14E are graphs illustrating the cytotoxicity (% of target cells killed) following co-culture of PBMCs and various doses of BiTE 5 (four replicates per dose) with target mesothelin-expressing cancer cell lines OVCAR-8 (A), A431/H9 (B), KLM-1 (C), RH29 (D), and HeLa (E).

FIG. 14F is a graph illustrating the cytotoxicity (% of target cells killed) following co-culture of T cells and various doses of BiTE 5 (2 replicates per dose) with target mesothelin-expressing cancer cell line KB31.

FIGS. 15A-15B are graphs illustrating the cytotoxicity (% of target cells killed) following co-culture of PBMCs and various doses of BiTE 5 (4 replicates per dose) with target cells mesothelin-positive pancreatic cancer cell line KLM-1 (A) or mesothelin-negative knock-out cell line KLM-1 KO #2 (B).

FIG. 16 is a graph showing tumor volume measured at various time points after KB31 and T cells from a healthy donor were co-implanted into NSG mice treated with BiTE 5 (2.5 mg/kg, closed circles) or PBS (open circles) (n=5). Upward pointing arrows indicate dosing.

### DETAILED DESCRIPTION OF THE INVENTION

Aspects of the invention provide polypeptides, proteins, and CARs which specifically recognize and bind to human mesothelin₅₈₂₋₅₉₈ (IPNGYLVLDLSMQEALS) (SEQ ID NO: 1). Mesothelin is expressed by normal, non-tumor, or non-cancerous mesothelial cells lining the pleura, peritoneum, and pericardium and is over-expressed by tumor or cancer cells from a variety of different cancers such as, e.g., ovarian cancer, pancreatic cancer, lung cancer (e.g., lung adenocarcinoma), esophageal cancer, gastric cancer, synovial sarcoma, and mesothelioma. The expression of mesothelin by normal, non-tumor, or non-cancerous cells is not as robust as the expression by tumor or cancer cells. In this regard, the tumor or cancer cells can over-express mesothelin or express mesothelin at a significantly higher level as compared to the expression of mesothelin by normal, non-tumor, or non-cancerous cells.

A challenge to the development of effective cancer immunotherapies targeting mesothelin is that mesothelin is shed from cancer cells in large amounts. For example, in mesothelioma and ovarian cancer, shed mesothelin levels may often be over about 0.1 μg/ml in blood and over about 1 μg/ml (e.g., as high as about 10 μg/ml) in ascites or pleural fluid. Without being bound to a particular theory or mechanism, it is also believed that high levels of shed mesothelin also exist inside solid tumors. The shedding of mesothelin from the cancer cells may remove anti-mesothelin immunotherapeutic agents that bind to the mesothelin on the cancer cells (such as, e.g., antibodies and CAR-T cells) and reduce the effectiveness of these and other anti-mesothelin immunotherapeutic agents. The shed mesothelin may also act as a decoy and reduce or prevent anti-mesothelin immunotherapeutic agents from binding to the cancer cells.

Monoclonal antibody (mAb) 15B6 specifically binds close to the plasma membrane at the C-terminus of mesothelin and blocks mesothelin shedding. Accordingly, mAb 15B6 may ameliorate some or all of the challenges associated with mesothelin shedding described above. However, mAb 15B6 is a murine antibody, which may be undesirably immunogenic when administered to a human patient.

The inventive polypeptides, proteins, and CARs advantageously provide humanized mAb 15B6 (hul5B6) antigen binding domains which may, advantageously, be less immunogenic when administered to a human patient and may also block mesothelin shedding. The inventive polypeptides and proteins were discovered after many common humanization strategies did not produce functional antigen binding domains. It was also unexpectedly discovered that only CARs comprising an antigen binding domain in the following format kills target cells: in order from the amino terminus to the carboxyl terminus, the VL CDR1 amino acid sequence, the VL CDR2 amino acid sequence, the VL CDR3 amino acid sequence, the VH CDR1 amino acid sequence, the VH CDR2 amino acid sequence, and the VH CDR3 amino acid sequence. In contrast, CARs comprising an antigen binding domain in the following format did not provide useful target cell killing ability: in order from the amino terminus to the carboxyl terminus, the VH CDR1 amino acid sequence, the VH CDR2 amino acid sequence, the VH CDR3 amino acid sequence, the VL CDR1 amino acid sequence, the VL CDR2 amino acid sequence, and the VL CDR3 amino acid sequence.

Without being bound to a particular theory or mechanism, it is believed that by specifically recognizing and binding to human mesothelin₅₈₂₋₅₉₈ (IPNGYLVLDLSMQEALS) (SEQ ID NO: 1), the inventive polypeptides, proteins, and CARs may, advantageously, reduce or prevent mesothelin shedding and improve the effectiveness of other anti-mesothelin immunotherapeutic agents. In an aspect of the invention, the inventive polypeptides, proteins, and CARs may elicit an antigen-specific response against mesothelin. Accordingly, without being bound to a particular theory or mechanism, it is believed that by specifically recognizing and binding mesothelin, the inventive polypeptides, proteins, and CARs may provide for one or more of the following: detecting mesothelin-expressing cancer cells, targeting and destroying mesothelin-expressing cancer cells, reducing or eliminating cancer cells, facilitating infiltration of immune cells and/or effector molecules to tumor site(s), and enhancing/extending anti-cancer responses.

The antigen binding domain of the inventive polypeptides, proteins, and CARs can be a whole antibody or an antibody fragment. A whole antibody typically consists of four polypeptides: two identical copies of a heavy (H) chain polypeptide and two identical copies of a light (L) chain polypeptide. Each of the heavy chains contains one N-terminal variable (VH) region and three C-terminal constant (CH1, CH2 and CH3) regions, and each light chain contains one N-terminal variable (VL) region and one C-terminal constant (CL) region. The variable regions of each pair of light and heavy chains form the antigen binding site of an antibody. The VH and VL regions have the same general structure, with each region comprising four framework (FR) regions, whose sequences are relatively conserved. The four VH FRs are referred to as VH FR1, VH FR2, VH FR3, and VH FR4. The four VL FRs are referred to as VL FR1, VL FR2, VL FR3, and VL FR4. The framework regions of each chain are connected by three complementarity determining regions (CDRs). The three VH CDRs are referred to as VH CDR1, VH CDR2, and VH CDR3. The three VL CDRs are referred to as VL CDR1, VL CDR2, and VL CDR3. The six CDRs form the “hypervariable region” of an antibody, which is responsible for antigen binding.

The terms “fragment of an antibody,” “antibody fragment.” “antigen binding domain,” and “antigen binding portion” are used interchangeably herein to mean one or more fragments or portions of an antibody that retain the ability to specifically bind to an antigen. The antigen binding domain of the inventive polypeptides, proteins, and CARs can contain any mesothelin-binding antibody fragment. The antibody fragment desirably comprises, for example, one or more CDRs, the variable region (or portions thereof), the constant region (or portions thereof), or combinations thereof.

The term “polypeptide.” as used herein, includes oligopeptides and refers to a single chain of amino acids connected by one or more peptide bonds. The polypeptide may comprise one or more variable regions (e.g., two variable regions) of an antigen binding domain of an anti-mesothelin antibody, each variable region comprising a CDR1, a CDR2,and a CDR3. In an aspect of the invention, the polypeptide comprises the CDR sequences of mAb 15B6 or humanized mAb 15B6. The CDR binding sequences may be determined by methods known in the art such as, for example, the methodology of the international ImMunoGeneTics information system (IMGT) or Kabat (Wu and Kabat J. Exp. Med., 132:211-250 (1970)).

An aspect of the invention provides a polypeptide which specifically binds to human mesothelin₅₈₂₋₅₉₈ (IPNGYLVLDLSMQEALS) (SEQ ID NO: 1) and which comprises:
(A) the light chain complementary determining region (VL CDR) 1 amino acid sequence of SEQ ID NO: 17 (hul5B6 Construct 9);
the VL CDR2 amino acid sequence of SEQ ID NO: 19 (hul5B6 Construct 9);
the VL CDR3 amino acid sequence of SEQ ID NO: 21 (hul5B6 Construct 9);
the heavy chain complementary determining region (VH CDR) 1 amino acid sequence of SEQ ID NO: 24 (hul5B6 Construct 9);
the VH CDR2 amino acid sequence of SEQ ID NO: 26 (hu15B6 Construct 9); and
the VH CDR3 amino acid sequence of SEQ ID NO: 28 (hu15B6 Construct 9);
(B) the VL CDR1 amino acid sequence of SEQ ID NO: 17 (hul5B6 Construct 10);
the VL CDR2 amino acid sequence of SEQ ID NO: 19 (hul5B6 Construct 10);
the VL CDR3 amino acid sequence of SEQ ID NO: 21 (hul5B6 Construct 10);
the VH CDR1 amino acid sequence of SEQ ID NO: 31 (hul5B6 Construct 10);
the VH CDR2 amino acid sequence of SEQ ID NO: 33 (hul5B6 Construct 10); and
the VH CDR3 amino acid sequence of SEQ ID NO: 35 (hu15B6 Construct 10);
(C) the VL CDR1 amino acid sequence of SEQ ID NO: 38 (hul5B6 Construct 11);
the VL CDR2 amino acid sequence of SEQ ID NO: 40 (hul5B6 Construct 11);
the VL CDR3 amino acid sequence of SEQ ID NO: 42 (hul5B6 Construct 11);
the VH CDR1 amino acid sequence of SEQ ID NO: 24 (hul5B6 Construct 11);
the VH CDR2 amino acid sequence of SEQ ID NO: 26 (hul5B6 Construct 11); and
the VH CDR3 amino acid sequence of SEQ ID NO: 28 (hul5B6 Construct 11); or
(D) the VL CDR1 amino acid sequence of SEQ ID NO: 38 (hu15B6 Construct 12);
the VL CDR2 amino acid sequence of SEQ ID NO: 40 (hul5B6 Construct 12);
the VL CDR3 amino acid sequence of SEQ ID NO: 42 (hul5B6 Construct 12);
the VH CDR1 amino acid sequence of SEQ ID NO: 31 (hul5B6 Construct 12);
the VH CDR2 amino acid sequence of SEQ ID NO: 33 (hul5B6 Construct 12); and
the VH CDR3 amino acid sequence of SEQ ID NO: 35 (hu15B6 Construct 12). The construct numbers in the foregoing paragraph refer to the Fv Construct Nos. of Table 5.

In an aspect of the invention, the polypeptide comprises the framework regions of each of the heavy and light chains, in addition to the CDRs of the heavy and light chains. In this regard, the polypeptide may comprise:
(A) the VL FR1 amino acid sequence of SEQ ID NO: 16 (hul5B6 Construct 9);
the VL CDR1 amino acid sequence of SEQ ID NO: 17 (hul5B6 Construct 9);
the VL FR2 amino acid sequence of SEQ ID NO: 18 (hu15B6 Construct 9);
the VL CDR2 amino acid sequence of SEQ ID NO: 19 (hul5B6 Construct 9);
the VL FR3 amino acid sequence of SEQ ID NO: 20 (hu15B6 Construct 9);
the VL CDR3 amino acid sequence of SEQ ID NO: 21 (hul5B6 Construct 9);
the VL FR4 amino acid sequence of SEQ ID NO: 22 (hu15B6 Construct 9);
the VH FR1 amino acid sequence of SEQ ID NO: 23 (hul5B6 Construct 9);
the VH CDR1 amino acid sequence of SEQ ID NO: 24 (hu15B6 Construct 9);
the VH FR2 amino acid sequence of SEQ ID NO: 25 (hu15B6 Construct 9);
the VH CDR2 amino acid sequence of SEQ ID NO: 26 (hu15B6 Construct 9);
the VH FR3 amino acid sequence of SEQ ID NO: 27 (hu15B6 Construct 9);
the VH CDR3 amino acid sequence of SEQ ID NO: 28 (hu15B6 Construct 9); and
the VH FR4 amino acid sequence of SEQ ID NO: 29 (hu15B6 Construct 9);
(B) the VL FR1 amino acid sequence of SEQ ID NO: 16 (hul5B6 Construct 10);
the VL CDR1 amino acid sequence of SEQ ID NO: 17 (hu15B6 Construct 10);
the VL FR2 amino acid sequence of SEQ ID NO: 18 (hu15B6 Construct 10);
the VL CDR2 amino acid sequence of SEQ ID NO: 19 (hul5B6 Construct 10);
the VL FR3 amino acid sequence of SEQ ID NO: 20 (hu15B6 Construct 10);
the VL CDR3 amino acid sequence of SEQ ID NO: 21 (hul5B6 Construct 10);
the VL FR4 amino acid sequence of SEQ ID NO: 22 (hu15B6 Construct 10);
the VH FR1 amino acid sequence of SEQ ID NO: 30 (hu15B6 Construct 10);
the VH CDR1 amino acid sequence of SEQ ID NO: 31 (hul5B6 Construct 10);
the VH FR2 amino acid sequence of SEQ ID NO: 32 (hu15B6 Construct 10);
the VH CDR2 amino acid sequence of SEQ ID NO: 33 (hul5B6 Construct 10);
the VH FR3 amino acid sequence of SEQ ID NO: 34 (hu15B6 Construct 10);
the VH CDR3 amino acid sequence of SEQ ID NO: 35 (hul5B6 Construct 10); and
the VH FR4 amino acid sequence of SEQ ID NO: 36 (hu15B6 Construct 10);
(C) the VL FR1 amino acid sequence of SEQ ID NO: 37 (hu15B6 Construct 11);
the VL CDR1 amino acid sequence of SEQ ID NO: 38 (hul5B6 Construct 11);
the VL FR2 amino acid sequence of SEQ ID NO: 39 (hu15B6 Construct 11);
the VL CDR2 amino acid sequence of SEQ ID NO: 40 (hul5B6 Construct 11);
the VL FR3 amino acid sequence of SEQ ID NO: 41 (hu15B6 Construct 11);
the VL CDR3 amino acid sequence of SEQ ID NO: 42 (hul5B6 Construct 11);
the VL FR4 amino acid sequence of SEQ ID NO: 43 (hu15B6 Construct 11);
the VH FR1 amino acid sequence of SEQ ID NO: 23 (hu15B6 Construct 11);
the VH CDR1 amino acid sequence of SEQ ID NO: 24 (hu15B6 Construct 11);
the VH FR2 amino acid sequence of SEQ ID NO: 25 (hu15B6 Construct 11);
the VH CDR2 amino acid sequence of SEQ ID NO: 26 (hu15B6 Construct 11);
the VH FR3 amino acid sequence of SEQ ID NO: 27 (hu15B6 Construct 11);
the VH CDR3 amino acid sequence of SEQ ID NO: 28 (hul5B6 Construct 11); and
the VH FR4 amino acid sequence of SEQ ID NO: 29 (hu15B6 Construct 11); or
(D) the VL FR1 amino acid sequence of SEQ ID NO: 37 (hul5B6 Construct 12);
the VL CDR1 amino acid sequence of SEQ ID NO: 38 (hul5B6 Construct 12);
the VL FR2 amino acid sequence of SEQ ID NO: 39 (hu15B6 Construct 12);
the VL CDR2 amino acid sequence of SEQ ID NO: 40 (hul5B6 Construct 12);
the VL FR3 amino acid sequence of SEQ ID NO: 41 (hu15B6 Construct 12);
the VL CDR3 amino acid sequence of SEQ ID NO: 42 (hul5B6 Construct 12);
the VL FR4 amino acid sequence of SEQ ID NO: 43 (hu15B6 Construct 12);
the VH FR1 amino acid sequence of SEQ ID NO: 30 (hu15B6 Construct 12);
the VH CDR1 amino acid sequence of SEQ ID NO: 31 (hu15B6 Construct 12);
the VH FR2 amino acid sequence of SEQ ID NO: 32 (hu15B6 Construct 12);
the VH CDR2 amino acid sequence of SEQ ID NO: 33 (hul5B6 Construct 12);
the VH FR3 amino acid sequence of SEQ ID NO: 34 (hu15B6 Construct 12);
the VH CDR3 amino acid sequence of SEQ ID NO: 35 (hul5B6 Construct 12); and
the VH FR4 amino acid sequence of SEQ ID NO: 36 (hul5B6 Construct 12). The construct numbers in the foregoing paragraph refer to the Fv Construct Nos. of Table 5.

In an aspect of the invention, the polypeptide may comprise a full length VH amino acid sequence comprising the VH CDRs and VH FRs described above and/or a full-length VL amino acid sequence comprising the VL CDRs and VL FRs described above. In this regard, the polypeptide may comprise:
(A) the VH amino acid sequence of SEQ ID NO: 46 and the VL amino acid sequence of SEQ ID NO: 48 (hu15B6 Construct 9);
(B) the VH amino acid sequence of SEQ ID NO: 47 and the VL amino acid sequence of SEQ ID NO: 48 (hu15B6 Construct 10);
(C) the VH amino acid sequence of SEQ ID NO: 46 and the VL amino acid sequence of SEQ ID NO: 49 (hu15B6 Construct 11); or
(D) the VH amino acid sequence of SEQ ID NO: 47 and the VL amino acid sequence of SEQ ID NO: 49 (hul5B6 Construct 12). The construct numbers in the foregoing paragraph refer to the Fv Construct Nos. of Table 5.

In some aspects, the polypeptide may comprise, in order from the amino terminus to the carboxyl terminus, the VH CDR1 amino acid sequence, the VH CDR2 amino acid sequence, the VH CDR3 amino acid sequence, the VL CDR1 amino acid sequence, the VL CDR2 amino acid sequence, and the VL CDR3 amino acid sequence. However, in a preferred aspect, the polypeptide comprises, in order from the amino terminus to the carboxyl terminus, the VL CDR1 amino acid sequence, the VL CDR2 amino acid sequence, the VL CDR3 amino acid sequence, the VH CDR1 amino acid sequence, the VH CDR2 amino acid sequence, and the VH CDR3 amino acid sequence.

In some aspects, the polypeptide may comprise, in order from the amino terminus to the carboxyl terminus, the VH amino acid sequence and the VL amino acid sequence. However, in a preferred aspect, the polypeptide comprises, in order from the amino terminus to the carboxyl terminus, the VL amino acid sequence and the VH amino acid sequence.

In an aspect of the invention, the variable regions of the polypeptide may be joined by a linker. The linker may comprise any suitable amino acid sequence. In an aspect of the invention, the linker is a Gly/Ser linker from about 1 to about 100, from about 3 to about 20, from about 5 to about 30, from about 5 to about 18, or from about 3 to about 8 amino acids in length and consists of glycine and/or serine residues in sequence. Accordingly, the Gly/Ser linker may consist of glycine and/or serine residues. In some aspects, the Gly/Ser linker is a peptide of the formula: (Xaa1)ₙ wherein each amino acid residue Xaa1 is selected independently from glycine and serine and n is an integer from 3 to 15.

An aspect of the invention further provides a protein comprising at least one of the polypeptides described herein. By “protein” is meant a molecule comprising one or more polypeptide chains.

An aspect of the invention provides a protein which specifically binds to human mesothelin₅₈₂₋₅₉₈ (IPNGYLVLDLSMQEALS) (SEQ ID NO: 1) and which comprises:
(A) a first polypeptide chain comprising the VL CDR1 amino acid sequence of SEQ ID NO: 17 (hu15B6 Construct 9), the VL CDR2 amino acid sequence of SEQ ID NO: 19 (hu15B6 Construct 9), and the VL CDR3 amino acid sequence of SEQ ID NO: 21 (hu15B6 Construct 9); and
a second polypeptide chain comprising the VH CDR1 amino acid sequence of SEQ ID NO: 24 (hu15B6 Construct 9), the VH CDR2 amino acid sequence of SEQ ID NO: 26 (hu15B6 Construct 9), and the VH CDR3 amino acid sequence of SEQ ID NO: 28 (hul5B6 Construct 9);
(B) a first polypeptide chain comprising the VL CDR1 amino acid sequence of SEQ ID NO: 17 (hul5B6 Construct 10), the VL CDR2 amino acid sequence of SEQ ID NO: 19 (hu15B6 Construct 10), the VL CDR3 amino acid sequence of SEQ ID NO: 21 (hu15B6 Construct 10), and
a second polypeptide chain comprising the VH CDR1 amino acid sequence of SEQ ID NO: 31 (hul5B6 Construct 10), the VH CDR2 amino acid sequence of SEQ ID NO: 33 (hu15B6 Construct 10), and the VH CDR3 amino acid sequence of SEQ ID NO: 35 (hul5B6 Construct 10);
(C) a first polypeptide chain comprising the VL CDR1 amino acid sequence of SEQ ID NO: 38 (hul5B6 Construct 11), the VL CDR2 amino acid sequence of SEQ ID NO: 40 (hu15B6 Construct 11), the VL CDR3 amino acid sequence of SEQ ID NO: 42 (hu15B6 Construct 11), and
a second polypeptide chain comprising the VH CDR1 amino acid sequence of SEQ ID NO: 24 (hul5B6 Construct 11), the VH CDR2 amino acid sequence of SEQ ID NO: 26 (hu15B6 Construct 11), and the VH CDR3 amino acid sequence of SEQ ID NO: 28 (hul5B6 Construct 11); or
(D) a first polypeptide chain comprising the VL CDR1 amino acid sequence of SEQ ID NO: 38 (hul5B6 Construct 12), the VL CDR2 amino acid sequence of SEQ ID NO: 40 (hu15B6 Construct 12), the VL CDR3 amino acid sequence of SEQ ID NO: 42 (hu15B6 Construct 12), and
a second polypeptide chain comprising the VH CDR1 amino acid sequence of SEQ ID NO: 31 (hul5B6 Construct 12), the VH CDR2 amino acid sequence of SEQ ID NO: 33 (hu15B6 Construct 12), and the VH CDR3 amino acid sequence of SEQ ID NO: 35 (hul5B6 Construct 12). The construct numbers in the foregoing paragraph refer to the Fv Construct Nos. of Table 5.

In an aspect of the invention, the protein comprises the framework regions of each of the heavy and light chains, in addition to the CDRs of the heavy and light chains. In this regard, the polypeptide may comprise:
(A) a first polypeptide chain comprising the VL FR1 amino acid sequence of SEQ ID NO: 16 (hul5B6 Construct 9), the VL CDR1 amino acid sequence of SEQ ID NO: 17 (hu15B6 Construct 9), the VL FR2 amino acid sequence of SEQ ID NO: 18 (hu15B6 Construct 9), the VL CDR2 amino acid sequence of SEQ ID NO: 19 (hul5B6 Construct 9), the VL FR3 amino acid sequence of SEQ ID NO: 20 (hu15B6 Construct 9), the VL CDR3 amino acid sequence of SEQ ID NO: 21 (hu15B6 Construct 9), the VL FR4 amino acid sequence of SEQ ID NO: 22 (hul5B6 Construct 9), and
a second polypeptide chain comprising the VH FR1 amino acid sequence of SEQ ID NO: 23 (hul5B6 Construct 9), the VH CDR1 amino acid sequence of SEQ ID NO: 24 (hu15B6 Construct 9), the VH FR2 amino acid sequence of SEQ ID NO: 25 (hu15B6 Construct 9), the VH CDR2 amino acid sequence of SEQ ID NO: 26 (hul5B6 Construct 9), the VH FR3 amino acid sequence of SEQ ID NO: 27 (hul5B6 Construct 9), the VH CDR3 amino acid sequence of SEQ ID NO: 28 (hul5B6 Construct 9), and the VH FR4 amino acid sequence of SEQ ID NO: 29 (hul5B6 Construct 9);
(B) a first polypeptide chain comprising the VL FR1 amino acid sequence of SEQ ID NO: 16 (hul5B6 Construct 10), the VL CDR1 amino acid sequence of SEQ ID NO: 17 (hu15B6 Construct 10), the VL FR2 amino acid sequence of SEQ ID NO: 18 (hu15B6 Construct 10), the VL CDR2 amino acid sequence of SEQ ID NO: 19 (hu15B6 Construct 10), the VL FR3 amino acid sequence of SEQ ID NO: 20 (hul5B6 Construct 10), the VL CDR3 amino acid sequence of SEQ ID NO: 21 (hul5B6 Construct 10), the VL FR4 amino acid sequence of SEQ ID NO: 22 (hul5B6 Construct 10); and
a second polypeptide chain comprising the VH FR1 amino acid sequence of SEQ ID NO: 30 (hul5B6 Construct 10), the VH CDR1 amino acid sequence of SEQ ID NO: 31 (hul5B6 Construct 10), the VH FR2 amino acid sequence of SEQ ID NO: 32 (hu15B6 Construct 10), the VH CDR2 amino acid sequence of SEQ ID NO: 33 (hu15B6 Construct 10), the VH FR3 amino acid sequence of SEQ ID NO: 34 (hul5B6 Construct 10), the VH CDR3 amino acid sequence of SEQ ID NO: 35 (hul5B6 Construct 10), and the VH FR4 amino acid sequence of SEQ ID NO: 36 (hu15B6 Construct 10);
(C) a first polypeptide chain comprising the VL FR1 amino acid sequence of SEQ ID NO: 37 (hul5B6 Construct 11), the VL CDR1 amino acid sequence of SEQ ID NO: 38 (hu15B6 Construct 11), the VL FR2 amino acid sequence of SEQ ID NO: 39 (hu15B6 Construct 11), the VL CDR2 amino acid sequence of SEQ ID NO: 40 (hu15B6 Construct 11), the VL FR3 amino acid sequence of SEQ ID NO: 41 (hul5B6 Construct 11), the VL CDR3 amino acid sequence of SEQ ID NO: 42 (hul5B6 Construct 11), the VL FR4 amino acid sequence of SEQ ID NO: 43 (hul5B6 Construct 11), and
a second polypeptide chain comprising the VH FR1 amino acid sequence of SEQ ID NO: 23 (hul5B6 Construct 11), the VH CDR1 amino acid sequence of SEQ ID NO: 24 (hu15B6 Construct 11), the VH FR2 amino acid sequence of SEQ ID NO: 25 (hu15B6 Construct 11), the VH CDR2 amino acid sequence of SEQ ID NO: 26 (hu15B6 Construct 11), the VH FR3 amino acid sequence of SEQ ID NO: 27 (hu15B6 Construct 11), the VH CDR3 amino acid sequence of SEQ ID NO: 28 (hul5B6 Construct 11), and the VH FR4 amino acid sequence of SEQ ID NO: 29 (hul5B6 Construct 11); or
(D) a first polypeptide chain comprising the VL FRI amino acid sequence of SEQ ID NO: 37 (hul5B6 Construct 12), the VL CDR1 amino acid sequence of SEQ ID NO: 38 (hu15B6 Construct 12), the VL FR2 amino acid sequence of SEQ ID NO: 39 (hu15B6 Construct 12), the VL CDR2 amino acid sequence of SEQ ID NO: 40 (hu15B6 Construct 12), the VL FR3 amino acid sequence of SEQ ID NO: 41 (hul5B6 Construct 12), the VL CDR3 amino acid sequence of SEQ ID NO: 42 (hul5B6 Construct 12), the VL FR4 amino acid sequence of SEQ ID NO: 43 (hul5B6 Construct 12), and
a second polypeptide chain comprising the VH FRI amino acid sequence of SEQ ID NO: 30 (hul5B6 Construct 12), the VH CDR1 amino acid sequence of SEQ ID NO: 31 (hu15B6 Construct 12), the VH FR2 amino acid sequence of SEQ ID NO: 32 (hu15B6 Construct 12), the VH CDR2 amino acid sequence of SEQ ID NO: 33 (hu15B6 Construct 12), the VH FR3 amino acid sequence of SEQ ID NO: 34 (hul5B6 Construct 12), the VH CDR3 amino acid sequence of SEQ ID NO: 35 (hul5B6 Construct 12), and the VH FR4 amino acid sequence of SEQ ID NO: 36 (hul5B6 Construct 12). The construct numbers in the foregoing paragraph refer to the Fv Construct Nos. of Table 5.

In an aspect of the invention, the protein may comprise a full length VH amino acid sequence comprising the VH CDRs and VH FRs described above and/or a full-length VL amino acid sequence comprising the VL CDRs and VL FRs described above. In this regard, the protein may comprise:
(A) a first polypeptide chain comprising the VH amino acid sequence of SEQ ID NO: 46 and a second polypeptide chain comprising the VL amino acid sequence of SEQ ID NO: 48 (hul5B6 Construct 9);
(B) a first polypeptide chain comprising the VH amino acid sequence of SEQ ID NO: 47 and a second polypeptide chain comprising the VL amino acid sequence of SEQ ID NO: 48 (hul5B6 Construct 10);
(C) a first polypeptide chain comprising the VH amino acid sequence of SEQ ID NO: 46 and a second polypeptide chain comprising the VL amino acid sequence of SEQ ID NO: 49 (hul5B6 Construct 11); or
(D) a first polypeptide chain comprising the VH amino acid sequence of SEQ ID NO: 47 and a second polypeptide chain comprising the VL amino acid sequence of SEQ ID NO: 49 (hu15B6 Construct 12). The construct numbers in the foregoing paragraph refer to the Fv Construct Nos. of Table 5.

The protein may further comprise a linker as described herein with respect to other aspects of the invention.

It is contemplated that the polypeptides and proteins of the invention may be useful as anti-mesothelin binding moieties. In this regard, an aspect of the invention provides an anti-mesothelin binding moiety comprising any of the polypeptides or proteins described herein. In an aspect of the invention, the anti-mesothelin binding moiety comprises an antigen binding portion of any of the polypeptides or proteins described herein. The antigen binding portion can be any portion that has at least one antigen binding site. In an aspect, the anti-mesothelin binding moiety is an antibody, Fab fragment (Fab), F(ab′)₂ fragment, diabody, triabody, tetrabody, multispecific antibody, single-chain variable region fragment (scFv), or disulfide-stabilized variable region fragment (dsFv). Preferably, the anti-mesothelin binding moiety is a scFv. In an aspect of the invention, the scFv comprises the amino acid sequence of any one of SEQ ID NOs: 50-51 and 58-61.

In an aspect, the anti-mesothelin binding moiety is an antibody. The antibody may be a monospecific antibody that has antigen specificity for only mesothelin or a multispecific antibody having antigen specificity for mesothelin and one or more other antigen(s) other than mesothelin. For example, the antibody may be a bispecific or trispecific antibody having antigen specificity for mesothelin and one or two other antigens other than mesothelin, respectively. The antibody may be, for example, a recombinant antibody comprising at least one of the inventive polypeptides described herein. As used herein, “recombinant antibody” refers to a recombinant (e.g., genetically engineered) protein comprising at least one of the polypeptides or proteins of the invention and one or more polypeptide chains of an antibody, or a portion thereof. The polypeptide of an antibody, or portion thereof, can be, for example, a constant region of a heavy or light chain, or an Fc fragment of an antibody, etc. The polypeptide chain of an antibody, or portion thereof, can exist as a separate polypeptide of the recombinant antibody. Alternatively, the polypeptide chain of an antibody, or portion thereof, can exist as a polypeptide, which is expressed in frame (in tandem) with the polypeptide or protein of the invention. The polypeptide of an antibody, or portion thereof, can be a polypeptide of any antibody or any antibody fragment.

The antibody of the invention can be any type of immunoglobulin that is known in the art. For instance, the anti-mesothelin binding moiety can be an antibody of any isotype, e.g., IgA, IgD, IgE, IgG (e.g., IgG1, IgG2, IgG3, or IgG4), IgM, etc. The antibody can be monoclonal or polyclonal. The antibody can be a naturally-occurring antibody, e.g., an antibody isolated and/or purified from a mammal, e.g., mouse, rabbit, goat, horse, chicken, hamster, human, etc. Alternatively, the antibody can be a genetically-engineered antibody, e.g., a humanized antibody or a chimeric antibody. The antibody can be in monomeric or polymeric form. Also, the antibody can have any level of affinity or avidity for mesothelin. Preferably, the antibody is a humanized antibody.

Methods of testing antibodies for the ability to bind to mesothelin are known in the art and include any antibody-antigen binding assay, such as, for example, radioimmunoassay (RIA), enzyme-linked immunosorbent assay (ELISA), Western blot, immunoprecipitation, and competitive inhibition assays.

Suitable methods of making antibodies are known in the art and include, for example, standard hybridoma methods, Epstein-Barr virus (EBV)-hybridoma methods, and bacteriophage vector expression systems. Antibodies may be produced in non-human animals.

In a preferred aspect, the anti-mesothelin binding moiety is an scFv. An scFv antibody fragment, which is a truncated Fab fragment including the variable (V) domain of an antibody heavy chain linked to a V domain of a light antibody chain via a synthetic peptide, can be generated using routine recombinant DNA technology techniques. Similarly, dsFvs can be prepared by recombinant DNA technology. The anti-mesothelin binding moieties of the invention, however, are not limited to these exemplary types of antibody fragments.

Also, the anti-mesothelin binding moiety can be modified to comprise a detectable label, such as, for instance, a radioisotope, a fluorophore (e.g., fluorescein isothiocyanate (FITC), phycoerythrin (PE)), an enzyme (e.g., alkaline phosphatase, horseradish peroxidase), and element particles (e.g., gold particles).

In an aspect of the invention, the anti-mesothelin binding moiety further comprises an agent which specifically binds to an immune cell. The immune cell may be a T cell or an NK cell. In this regard, the agent which specifically binds to the immune cell may be a T cell engager or an NK cell engager. Examples of such agents include, but are not limited to, a bispecific T cell engager, a bispecific NK cell engager, a trispecific T cell engager, or a trispecific NK cell engager. In an aspect of the invention, the anti-mesothelin binding moiety may comprise knobs-into-holes (KiH) mutations, e.g., in the Fc region.

Bispecific NK cell engagers and trispecific NK cell engagers (BiKEs and TriKEs, respectively) target NK cells to the tumor synapse and induce activation of the NK cell at that site. BiKEs and TriKEs are small molecules containing an antigen binding domain of an antibody that specifically binds to an NK cell marker (e.g., CD16) linked to one (BiKE) or two (TriKE) antigen binding domain(s) from other antibodies of different specificity (e.g., for cancer specific antigens). For example, a BiKE or TriKE may bind to NK cells and the target cancer cell(s), resulting in the formation of an immunological synapse, which triggers NK killing of the target cancer cell(s) through activation of the low affinity Fc receptor, CD16, on NK cells. An example of a trispecific NK cell engager is HER2 tri-specific NK cell engager DF1001. HER2 tri-specific NK cell engager DF1001 targets and binds to HER2 on tumor cells and simultaneously binds to NK cells, thereby bringing HER2-expressing tumor cells and NK cells together, which stimulates the NK cells and results in the selective NK cell-mediated tumor cell lysis of HER2-expressing tumor cells.

Bispecific T cell engagers (BiTEs) and trispecific T cell engagers are fusion proteins with two (BiTE) or three (trispecific T cell engager) antigen binding domains of different antibodies on a single peptide chain. One of the antigen binding domains specifically binds to T cells via a T-cell-specific marker (e.g., the CD3 receptor), and the other one or two antigen binding domain(s) specifically bind(s) to cancer cell(s) via cancer specific antigen(s). BiTEs and trispecific T cell engagers form a link between T cells and cancer cells. The T cells then exert cytotoxic activity on cancer cells, e.g., by initiating the cancer cell's apoptosis. In an aspect of the invention, the anti-mesothelin binding moiety is a BiTE. In an aspect of the invention, the BiTE comprises (i) the amino acid sequence of SEQ ID NO: 97, (ii) the amino acid sequence of SEQ ID NO: 98, (iii) the amino acid sequence of SEQ ID NO: 99, or (iv) the amino acid sequences of all of SEQ ID NO: 100, SEQ ID NO: 101, SEQ ID NO: 102, and SEQ ID NO: 103.

Included in the scope of the aspects of the invention are conjugates, e.g., bioconjugates, comprising any of the inventive polypeptides, proteins, anti-mesothelin binding moieties, functional portions, or functional variants thereof. Conjugates, as well as methods of synthesizing conjugates in general, are known in the art. In this regard, an aspect of the invention provides a conjugate comprising (a) any of the polypeptides, proteins, or anti-mesothelin binding moieties described herein conjugated or fused to (b) an effector molecule. The effector molecule may be any therapeutic molecule or a molecule that facilitates the detection of the conjugate such as a drug, toxin, label (e.g., any of the detectable labels described herein), small molecule, or another antibody. For example, the toxin may be Pseudomonas exotoxin A (“PE”) or variants thereof such as, e.g., any of PE24, PE4E, PE40, PE38, PE25, PE38QQR, PE38KDEL, PE-LR, and PE35, as described in, e.g., U.S. Pat. Nos. 4,892,827; 5,512,658; 5,602,095; 5,608,039; 5,821,238; 5,854,044; and 8,871,906, each of which is incorporated herein by reference. PE variants include PE which has been modified to remove B cell and/or T-cell epitopes to reduce the immunogenicity of the PE as described in, for example, any of U.S. Pat. Nos. 9,206,240; 9,346,859; 9,388,222; 8,907,060; 8,871,906; 8,936,792; and 10,683,362, each of which is incorporated herein by reference. PE is a bacterial toxin with cytotoxic activity that may be effective for destroying or inhibiting the growth of undesirable cells, e.g., cancer cells. Accordingly, PE may be useful for treating or preventing diseases such as cancer.

Examples of drugs that may be suitable in the inventive conjugates include, but are not limited to, pyrrolobenzodiazepine (PBD) dimer, tubulin-binders such as, for example, dolastatin 10, monomethyl dolastatin 10, auristain E, monomethyl auristain E (MMAE), auristatin F, monomethyl auristatin F, HTI-286, tubulysin M, maytansinoid AP-3, cryptophycin, Boc-Val-Dil-Dap-OH, tubulysin IM-1, Boc-Val-Dil-Dap-Phe-OMe, tubulysin IM-2, Boc-Nme-Val-Val-Dil-Dap-OH, tubulysin IM-3, and colchicine DA: DNA-alkylators(duocarmycin analogs) such as, for example, duocarmycin SA, duocarmycin CN, duocarmycin DMG, duocarmycin DMA, duocarmycin MA, duocarmycin TM, duocarmycin MB, duocarmycin GA; tomaymycin DM; SJG-136; illudin S; irofulven; apaziquone; triptolide; staurosporine; camptothecin; methotrexate; and other anti-cancer drugs such as, for example, kinase inhibitors, histone deacetylase (HDAC) inhibitors, proteasome inhibitors, and matrix metalloproteinase (MMP) inhibitors. In an aspect, the drug is MMAE or PBD dimer.

The polypeptides, proteins, or anti-mesothelin binding moieties described herein may be conjugated or fused to (b) an effector molecule (such as a drug, toxin, label, small molecule, or an antibody) directly or indirectly, e.g., via a linking moiety. The linking moiety may be any suitable linking moiety known in the art. In an aspect, the linking moiety is a comprises a cleavage sequence that may be cleaved upon administration of the conjugate to a mammal. Cleavage sequences are known in the art and include, but are not limited to, 2A self-cleaving peptides. Examples of 2A self-cleaving peptides include those derived from P2A, E2A, F2A, and T2A.

Another aspect of the invention provides CARs comprising: (a) an antigen binding domain comprising any of the polypeptides, proteins, or anti-mesothelin binding moieties described herein, wherein the antigen binding domain has antigen specificity for human mesothelin₅₈₂₋₅₉₈ (IPNGYLVLDLSMQEALS) (SEQ ID NO: 1), (b) a TM domain, and (c) an intracellular T cell signaling domain.

A CAR is an artificially constructed hybrid protein or polypeptide containing the antigen binding domains of an antibody (e.g., scFv) linked to T-cell signaling domains. Characteristics of CARs include their ability to redirect T-cell specificity and reactivity toward a selected target in a non-MHC-restricted manner, exploiting the antigen-binding properties of monoclonal antibodies. The non-MHC-restricted antigen recognition gives cells expressing CARs the ability to recognize antigen independent of antigen processing, thus bypassing a major mechanism of tumor escape. Moreover, when expressed in T-cells, CARs advantageously do not dimerize with endogenous T cell receptor (TCR) alpha and beta chains.

The phrases “have antigen specificity” and “elicit antigen-specific response,” as used herein, means that the CAR can specifically bind to and immunologically recognize an antigen, such that binding of the CAR to the antigen elicits an immune response.

The CARs of the invention have antigen specificity for mesothelin. Without being bound to a particular theory or mechanism, it is believed that by eliciting an antigen-specific response against mesothelin, the inventive CARs provide for one or more of the following: targeting and destroying mesothelin-expressing cancer cells, reducing or eliminating cancer cells, facilitating infiltration of immune cells to tumor site(s), and enhancing/extending anti-cancer responses.

An aspect of the invention provides a CAR comprising an antigen binding domain of an anti-mesothelin antibody. The antigen binding domain of the anti-mesothelin antibody specifically binds to mesothelin. The antigen binding domain of the CARs may comprise any of the polypeptides, proteins, or anti-mesothelin binding moieties described herein. In an aspect of the invention, the CAR comprises an anti-mesothelin scFv. In this regard, a preferred aspect of the invention provides a CAR comprising an antigen-binding domain comprising an scFv that comprises any of the polypeptides or proteins described herein.

In an aspect of the invention, the antigen binding domain of the CAR comprises:
(A) the VL CDR1 amino acid sequence of SEQ ID NO: 17 (hu15B6 Construct 9);
the VL CDR2 amino acid sequence of SEQ ID NO: 19 (hul5B6 Construct 9);
the VL CDR3 amino acid sequence of SEQ ID NO: 21 (hul5B6 Construct 9);
the VH CDR1 amino acid sequence of SEQ ID NO: 24 (hu15B6 Construct 9);
the VH CDR2 amino acid sequence of SEQ ID NO: 26 (hu15B6 Construct 9); and
the VH CDR3 amino acid sequence of SEQ ID NO: 28 (hu15B6 Construct 9);
(B) the VL CDR1 amino acid sequence of SEQ ID NO: 17 (hul5B6 Construct 10);
the VL CDR2 amino acid sequence of SEQ ID NO: 19 (hul5B6 Construct 10);
the VL CDR3 amino acid sequence of SEQ ID NO: 21 (hul5B6 Construct 10);
the VH CDR1 amino acid sequence of SEQ ID NO: 31 (hul5B6 Construct 10);
the VH CDR2 amino acid sequence of SEQ ID NO: 33 (hul5B6 Construct 10); and
the VH CDR3 amino acid sequence of SEQ ID NO: 35 (hu15B6 Construct 10);
(C) the VL CDR1 amino acid sequence of SEQ ID NO: 38 (hul5B6 Construct 11);
the VL CDR2 amino acid sequence of SEQ ID NO: 40 (hul5B6 Construct 11);
the VL CDR3 amino acid sequence of SEQ ID NO: 42 (hul5B6 Construct 11);
the VH CDR1 amino acid sequence of SEQ ID NO: 24 (hul5B6 Construct 11);
the VH CDR2 amino acid sequence of SEQ ID NO: 26 (hul5B6 Construct 11); and
the VH CDR3 amino acid sequence of SEQ ID NO: 28 (hul5B6 Construct 11);
(D) the VL CDR1 amino acid sequence of SEQ ID NO: 38 (hu15B6 Construct 12);
the VL CDR2 amino acid sequence of SEQ ID NO: 40 (hul5B6 Construct 12);
the VL CDR3 amino acid sequence of SEQ ID NO: 42 (hul5B6 Construct 12);
the VH CDR1 amino acid sequence of SEQ ID NO: 31 (hul5B6 Construct 12);
the VH CDR2 amino acid sequence of SEQ ID NO: 33 (hul5B6 Construct 12); and
the VH CDR3 amino acid sequence of SEQ ID NO: 35 (hul5B6 Construct 12); or
(E) the VL CDR1 amino acid sequence of SEQ ID NO: 3 (15B6 scFv Constructs 1 and 2);
the VL CDR2 amino acid sequence of SEQ ID NO: 5 (15B6 scFv Constructs 1 and 2);
the VL CDR3 amino acid sequence of SEQ ID NO: 7 (15B6 scFv Constructs 1 and 2);
the VH CDR1 amino acid sequence of SEQ ID NO: 10 (15B6 scFv Constructs 1 and 2);
the VH CDR2 amino acid sequence of SEQ ID NO: 12 (15B6 scFv Constructs 1 and 2); and
the VH CDR3 amino acid sequence of SEQ ID NO: 14 (15B6 scFv Constructs 1 and 2). The construct numbers in the foregoing paragraph refer to the Fv Construct Nos. of Tables 2 and 5.

In an aspect of the invention, the antigen binding domain of the CAR comprises the framework regions of each of the heavy and light chains, in addition to the CDRs of the heavy and light chains. In this regard, the antigen binding domain of the CAR may comprise:
(A) the VL FRI amino acid sequence of SEQ ID NO: 16 (hul5B6 Construct 9);
the VL CDR1 amino acid sequence of SEQ ID NO: 17 (hul5B6 Construct 9);
the VL FR2 amino acid sequence of SEQ ID NO: 18 (hu15B6 Construct 9);
the VL CDR2 amino acid sequence of SEQ ID NO: 19 (hul5B6 Construct 9);
the VL FR3 amino acid sequence of SEQ ID NO: 20 (hu15B6 Construct 9);
the VL CDR3 amino acid sequence of SEQ ID NO: 21 (hul5B6 Construct 9);
the VL FR4 amino acid sequence of SEQ ID NO: 22 (hu15B6 Construct 9);
the VH FR1 amino acid sequence of SEQ ID NO: 23 (hu15B6 Construct 9);
the VH CDR1 amino acid sequence of SEQ ID NO: 24 (hu15B6 Construct 9);
the VH FR2 amino acid sequence of SEQ ID NO: 25 (hu15B6 Construct 9);
the VH CDR2 amino acid sequence of SEQ ID NO: 26 (hu15B6 Construct 9);
the VH FR3 amino acid sequence of SEQ ID NO: 27 (hu15B6 Construct 9);
the VH CDR3 amino acid sequence of SEQ ID NO: 28 (hu15B6 Construct 9); and
the VH FR4 amino acid sequence of SEQ ID NO: 29 (hu15B6 Construct 9);
(B) the VL FR1 amino acid sequence of SEQ ID NO: 16 (hu15B6 Construct 10);
the VL CDR1 amino acid sequence of SEQ ID NO: 17 (hul5B6 Construct 10);
the VL FR2 amino acid sequence of SEQ ID NO: 18 (hu15B6 Construct 10);
the VL CDR2 amino acid sequence of SEQ ID NO: 19 (hul5B6 Construct 10);
the VL FR3 amino acid sequence of SEQ ID NO: 20 (hu15B6 Construct 10);
the VL CDR3 amino acid sequence of SEQ ID NO: 21 (hul5B6 Construct 10);
the VL FR4 amino acid sequence of SEQ ID NO: 22 (hu15B6 Construct 10);
the VH FR1 amino acid sequence of SEQ ID NO: 30 (hu15B6 Construct 10);
the VH CDR1 amino acid sequence of SEQ ID NO: 31 (hul5B6 Construct 10);
the VH FR2 amino acid sequence of SEQ ID NO: 32 (hu15B6 Construct 10);
the VH CDR2 amino acid sequence of SEQ ID NO: 33 (hul5B6 Construct 10);
the VH FR3 amino acid sequence of SEQ ID NO: 34 (hu15B6 Construct 10);
the VH CDR3 amino acid sequence of SEQ ID NO: 35 (hul5B6 Construct 10); and
the VH FR4 amino acid sequence of SEQ ID NO: 36 (hu15B6 Construct 10);
(C) the VL FR1 amino acid sequence of SEQ ID NO: 37 (hu15B6 Construct 11);
the VL CDR1 amino acid sequence of SEQ ID NO: 38 (hul5B6 Construct 11);
the VL FR2 amino acid sequence of SEQ ID NO: 39 (hu15B6 Construct 11);
the VL CDR2 amino acid sequence of SEQ ID NO: 40 (hul5B6 Construct 11);
the VL FR3 amino acid sequence of SEQ ID NO: 41 (hu15B6 Construct 11);
the VL CDR3 amino acid sequence of SEQ ID NO: 42 (hul5B6 Construct 11);
the VL FR4 amino acid sequence of SEQ ID NO: 43 (hu15B6 Construct 11);
the VH FR1 amino acid sequence of SEQ ID NO: 23 (hu15B6 Construct 11);
the VH CDR1 amino acid sequence of SEQ ID NO: 24 (hul5B6 Construct 11);
the VH FR2 amino acid sequence of SEQ ID NO: 25 (hu15B6 Construct 11);
the VH CDR2 amino acid sequence of SEQ ID NO: 26 (hul5B6 Construct 11);
the VH FR3 amino acid sequence of SEQ ID NO: 27 (hu15B6 Construct 11);
the VH CDR3 amino acid sequence of SEQ ID NO: 28 (hul5B6 Construct 11); and
the VH FR4 amino acid sequence of SEQ ID NO: 29 (hu15B6 Construct 11);
(D) the VL FR1 amino acid sequence of SEQ ID NO: 37 (hul5B6 Construct 12);
the VL CDR1 amino acid sequence of SEQ ID NO: 38 (hul5B6 Construct 12);
the VL FR2 amino acid sequence of SEQ ID NO: 39 (hu15B6 Construct 12);
the VL CDR2 amino acid sequence of SEQ ID NO: 40 (hul5B6 Construct 12);
the VL FR3 amino acid sequence of SEQ ID NO: 41 (hu15B6 Construct 12);
the VL CDR3 amino acid sequence of SEQ ID NO: 42 (hul5B6 Construct 12);
the VL FR4 amino acid sequence of SEQ ID NO: 43 (hu15B6 Construct 12);
the VH FR1 amino acid sequence of SEQ ID NO: 30 (hu15B6 Construct 12);
the VH CDR1 amino acid sequence of SEQ ID NO: 31 (hul5B6 Construct 12);
the VH FR2 amino acid sequence of SEQ ID NO: 32 (hu15B6 Construct 12);
the VH CDR2 amino acid sequence of SEQ ID NO: 33 (hul5B6 Construct 12);
the VH FR3 amino acid sequence of SEQ ID NO: 34 (hu15B6 Construct 12);
the VH CDR3 amino acid sequence of SEQ ID NO: 35 (hul5B6 Construct 12); and
the VH FR4 amino acid sequence of SEQ ID NO: 36 (hu15B6 Construct 12); or
(E) the VL FF1 amino acid sequence of SEQ ID NO: 2 (15B6 scFv Constructs 1 and 2);
the VL CDR1 amino acid sequence of SEQ ID NO: 3 (15B6 scFv Constructs 1 and 2);
the VL FR2 amino acid sequence of SEQ ID NO: 4 (15B6 scFv Constructs 1 and 2);
the VL CDR2 amino acid sequence of SEQ ID NO: 5 (15B6 scFv Constructs 1 and 2);
the VL FR3 amino acid sequence of SEQ ID NO: 6 (15B6 scFv Constructs 1 and 2);
the VL CDR3 amino acid sequence of SEQ ID NO: 7 (15B6 scFv Constructs 1 and 2);
the VL FR4 amino acid sequence of SEQ ID NO: 8 (15B6 scFv Constructs 1 and 2);
the VH FR1 amino acid sequence of SEQ ID NO: 9 (15B6 scFv Constructs 1 and 2);
the VH CDR1 amino acid sequence of SEQ ID NO: 10 (15B6 scFv Constructs 1 and 2);
the VH FR2 amino acid sequence of SEQ ID NO: 11 (15B6 scFv Constructs 1 and 2);
the VH CDR2 amino acid sequence of SEQ ID NO: 12 (15B6 scFv Constructs 1 and 2);
the VH FR3 amino acid sequence of SEQ ID NO: 13 (15B6 scFv Constructs 1 and 2);
the VH CDR3 amino acid sequence of SEQ ID NO: 14 (15B6 scFv Constructs 1 and 2); and
the VH FR4 amino acid sequence of SEQ ID NO: 15 (15B6 scFv Constructs 1 and 2). The construct numbers in the foregoing paragraph refer to the Fv Construct Nos. of Tables 2 and 5.

In an aspect of the invention, the antigen binding domain of the CAR may comprise a full length VH amino acid sequence comprising the VH CDRs and VH FRs described above and/or a full-length VL amino acid sequence comprising the VL CDRs and VL FRs described above. In this regard, the antigen binding domain of the CAR may comprise:
(A) the VH amino acid sequence of SEQ ID NO: 46 and the VL amino acid sequence of SEQ ID NO: 48 (hu15B6 Construct 9);
(B) the VH amino acid sequence of SEQ ID NO: 47 and the VL amino acid sequence of SEQ ID NO: 48 (hu15B6 Construct 10);
(C) the VH amino acid sequence of SEQ ID NO: 46 and the VL amino acid sequence of SEQ ID NO: 49 (hu15B6 Construct 11);
(D) the VH amino acid sequence of SEQ ID NO: 47 and the VL amino acid sequence of SEQ ID NO: 49 (hul5B6 Construct 12); or
(E) the VH amino acid sequence of SEQ ID NO: 44 and the VL amino acid sequence of SEQ ID NO: 45. The construct numbers in the foregoing paragraph refer to the Fv Construct Nos. of Tables 2 and 5.

In some aspects, the antigen binding domain of the CAR may comprise, in order from the amino terminus to the carboxyl terminus, the VH CDR1 amino acid sequence, the VH CDR2 amino acid sequence, the VH CDR3 amino acid sequence, the VL CDR1 amino acid sequence, the VL CDR2 amino acid sequence, and the VL CDR3 amino acid sequence. However, in a preferred aspect, the antigen binding domain of the CAR comprises, in order from the amino terminus to the carboxyl terminus, the VL CDR1 amino acid sequence, the VL CDR2 amino acid sequence, the VL CDR3 amino acid sequence, the VH CDR1 amino acid sequence, the VH CDR2 amino acid sequence, and the VH CDR3 amino acid sequence.

In some aspects, the antigen binding domain of the CAR may comprise, in order from the amino terminus to the carboxyl terminus, the VH amino acid sequence and the VL amino acid sequence. However, in a preferred aspect, the antigen binding domain of the CAR comprises, in order from the amino terminus to the carboxyl terminus, the VL amino acid sequence and the VH amino acid sequence.

In an aspect of the invention, the antigen binding domain of the CAR comprises the amino acid sequence of any one of SEQ ID NOs: 50-51 and 58-61.

In an aspect, the antigen binding domain of the CAR comprises a leader (signal) sequence. In an aspect of the invention, while the leader sequence may facilitate expression of the CAR on the surface of the cell, the presence of the leader sequence in an expressed CAR is not necessary in order for the CAR to function. In an aspect of the invention, upon expression of the CAR on the cell surface, the leader sequence may be cleaved off of the CAR. Accordingly, in an aspect of the invention, the CAR lacks a leader sequence.

In an aspect, the CAR comprises a hinge domain. The hinge domain may comprise, for example, a hinge domain of any one of the following proteins: a CD8 protein, a CD28 protein, a IgG1 protein, or a IgG4 protein. Without being bound to a particular theory, it is believed that the hinge domain extends the binding motif of the antigen binding domain away from the membrane of the CAR-expressing cells and may more accurately mimic the size and domain structure of a native TCR. In some aspects, the CAR may lack a hinge domain. In an aspect of the invention, the hinge domain comprises the hinge domain of a human protein.

In an aspect of the invention, the CAR comprises a TM domain. In an aspect of the invention, the TM domain comprises any one of the following: a CD3 zeta TM domain, a CD4 TM domain, a CD8 TM domain, a CD28 TM domain, a ICOS TM domain, or any combination of the foregoing. In a preferred aspect, the TM domain is a human TM domain.

In an aspect of the invention, the CAR comprises an intracellular T cell signaling domain. The intracellular T cell signaling domain may comprise the intracellular T cell signaling domain of any one of the following proteins: a CD3-zeta protein, a CD27 protein, a CD28 protein, a CD40) protein, a FcRγ protein, an inducible T-cell costimulatory protein (ICOS), a killer cell immunoglobulin-like receptor 2DS2 protein (KIR2DS2), a MYD88 protein, a OX40 protein, a 4-1BB protein, or any combination of the foregoing. In a preferred aspect, the intracellular T cell signaling domain is human. CD28 is a T cell marker important in T cell co-stimulation. CD137, also known as 4-1BB, transmits a potent costimulatory signal to T cells, promoting differentiation and enhancing long-term survival of T lymphocytes. CD3ζ associates with TCRs to produce a signal and contains immunoreceptor tyrosine-based activation motifs (ITAMs).

In an aspect of the invention, the CAR comprises the amino acid sequence of any one of SEQ ID NOs: 70-77, 86-87, and 90-91.

In an aspect of the invention, any of the inventive polypeptides, proteins, anti-mesothelin binding moieties, conjugates, and CARs described herein may be biparatopic polypeptides, biparatopic proteins, biparatopic anti-mesothelin binding moieties, biparatopic conjugates, and biparatopic CARs, respectively. Biparatopic agents have two antigen binding domains, wherein each antigen binding domain recognizes unique, non-overlapping epitopes on the same target antigen. In an aspect of the invention, any of the polypeptides, proteins, anti-mesothelin binding moieties, conjugates, and CARs described herein may provide biparatopic polypeptides, biparatopic proteins, biparatopic anti-mesothelin binding moieties, biparatopic conjugates, and biparatopic CARs which specifically bind to human mesothelin₅₈₂₋₅₉₈ (IPNGYLVLDLSMQEALS) (SEQ ID NO: 1) and another human mesothelin epitope other than human mesothelin₅₈₂₋₅₉₈ (IPNGYLVLDLSMQEALS) (SEQ ID NO: 1). Such biparatopic agents may target and kill mesothelin-expressing cells more effectively by both blocking mesothelin shedding and also specifically binding to another mesothelin epitope.

An aspect of the invention provides a bispecific, biparatopic CAR comprising a first antigen binding domain comprising any of the inventive polypeptides, proteins, or anti-mesothelin binding moieties described herein with respect to other aspects of the invention, and a second antigen binding domain having antigen specificity for a human mesothelin epitope other than human mesothelin₅₈₂₋₅₉₈ (IPNGYLVLDLSMQEALS) (SEQ ID NO: 1).

The second antigen binding domain having antigen specificity for a human mesothelin epitope other than human mesothelin₅₈₂₋₅₉₈ (IPNGYLVLDLSMQEALS) (SEQ ID NO: 1) may comprise the antigen binding domain of any anti-mesothelin antibody known in the art having antigen specificity for a human mesothelin epitope other than human mesothelin₅₈₂₋₅₉₈ (IPNGYLVLDLSMQEALS) (SEQ ID NO: 1). Several mesothelin-specific monoclonal antibodies are known in the art, including, but not limited to YP218 disclosed in U.S. Pat. No. 9,803,022 and U.S. Patent Application Publication No. 2021/0371542; YP223, YP3, YP158 and YP187 disclosed in U.S. Pat. No. 9,803,022; SD1 disclosed in U.S. Pat. No. 9,416,190 and U.S. Patent Application Publication No. 2021/0371542; HN1 disclosed in U.S. Pat. No. 8,460,660; SS disclosed in U.S. Patent No. 6,809, 184; and SS1 disclosed in U.S. Pat. No. 7,081,518, each of which are incorporated herein by reference in their entirety. In an aspect of the invention, the second antigen binding domain of the bispecific, biparatopic CAR comprises the antigen binding domain of mAb YP218 or humanized mAb YP218. Antigen binding domains of mAb YP218 and humanized mAb YP218 are described, e.g., in U.S. Pat. No. 9,803,022 and U.S. Patent Application Publication No. 2021/0371542, each of which are incorporated herein by reference in their entirety.

Included in the scope of the invention are functional portions of the inventive polypeptides, proteins, and CARs described herein. The term “functional portion,” when used in reference to a polypeptide, protein, or CAR, refers to any part or fragment of the polypeptide, protein, or CAR of the invention, which part or fragment retains the biological activity of the polypeptide, protein, or CAR of which it is a part (the parent polypeptide, protein, or CAR). Functional portions encompass, for example, those parts of a polypeptide, protein, or CAR that retain the ability to recognize target cells, or detect, treat, or prevent cancer, to a similar extent, the same extent, or to a higher extent, as the parent polypeptide, protein, or CAR. In reference to the parent polypeptide, protein, or CAR, the functional portion can comprise, for instance, about 10%, 25%, 30%, 50%, 68%, 80%, 90%, 95%, or more, of the parent polypeptide, protein, or CAR.

The functional portion can comprise additional amino acids at the amino or carboxy terminus of the portion, or at both termini, which additional amino acids are not found in the amino acid sequence of the parent polypeptide, protein, or CAR. Desirably, the additional amino acids do not interfere with the biological function of the functional portion, e.g., recognize target cells, detect cancer, treat or prevent cancer, etc. More desirably, the additional amino acids enhance the biological activity, as compared to the biological activity of the parent polypeptide, protein, or CAR.

Included in the scope of the invention are functional variants of the inventive polypeptides, proteins, or CARs described herein. The term “functional variant.” as used herein, refers to a polypeptide, protein, or CAR having substantial or significant sequence identity or similarity to a parent polypeptide, protein, or CAR, which functional variant retains the biological activity of the polypeptide, protein, or CAR of which it is a variant. Functional variants encompass, for example, those variants of the polypeptide, protein, or CAR described herein (the parent polypeptide, protein, or CAR) that retain the ability to recognize target cells to a similar extent, the same extent, or to a higher extent, as the parent polypeptide, protein, or CAR. In reference to the parent polypeptide, protein, or CAR, the functional variant can, for instance, be at least about 30%, about 50%, about 75%, about 80%, about 85%, about 90%, about 91%, about 92%, about 93%, about 94%, about 95%, about 96%, about 97%, about 98%, about 99% or more identical in amino acid sequence to the parent polypeptide, protein, or CAR.

A functional variant can, for example, comprise the amino acid sequence of the parent polypeptide, protein, or CAR with at least one conservative amino acid substitution. Alternatively or additionally, the functional variants can comprise the amino acid sequence of the parent polypeptide, protein, or CAR with at least one non-conservative amino acid substitution. In this case, it is preferable for the non-conservative amino acid substitution to not interfere with or inhibit the biological activity of the functional variant. The non-conservative amino acid substitution may enhance the biological activity of the functional variant, such that the biological activity of the functional variant is increased as compared to the parent polypeptide, protein, or CAR.

Amino acid substitutions of the inventive polypeptides, proteins, or CARs are preferably conservative amino acid substitutions. Conservative amino acid substitutions are known in the art, and include amino acid substitutions in which one amino acid having certain physical and/or chemical properties is exchanged for another amino acid that has the same or similar chemical or physical properties. For instance, the conservative amino acid substitution can be an acidic/negatively charged polar amino acid substituted for another acidic/negatively charged polar amino acid (e.g., Asp or Glu), an amino acid with a nonpolar side chain substituted for another amino acid with a nonpolar side chain (e.g., Ala, Gly, Val, Ile, Leu, Met, Phe, Pro, Trp, Cys, Val, etc.), a basic/positively charged polar amino acid substituted for another basic/positively charged polar amino acid (e.g. Lys, His, Arg, etc.), an uncharged amino acid with a polar side chain substituted for another uncharged amino acid with a polar side chain (e.g., Asn, Gln, Ser, Thr, Tyr, etc.), an amino acid with a beta-branched side-chain substituted for another amino acid with a beta-branched side-chain (e.g., Ile, Thr, and Val), an amino acid with an aromatic side-chain substituted for another amino acid with an aromatic side chain (e.g., His, Phe, Trp, and Tyr), etc.

The polypeptide, protein, or CAR can consist essentially of the specified amino acid sequence or sequences described herein, such that other components, e.g., other amino acids, do not materially change the biological activity of the polypeptide, protein, CAR, functional portion, or functional variant.

The polypeptides, proteins, or CARs of aspects of the invention (including functional portions and functional variants) can be of any length, i.e., can comprise any number of amino acids, provided that the polypeptides, proteins, or CARs (or functional portions or functional variants thereof) retain their biological activity, e.g., the ability to specifically bind to antigen, detect cancer cells in a mammal, or treat or prevent cancer in a mammal, etc. For example, the polypeptide, protein, or CAR can be about 50 to about 5000 amino acids long, such as 50, 70, 75, 100, 125, 150, 175, 200, 300, 400, 500, 600, 700, 800, 900, 1000 or more amino acids in length.

The polypeptides, proteins, or CARs of aspects of the invention (including functional portions and functional variants of the invention) can comprise synthetic amino acids in place of one or more naturally-occurring amino acids. Such synthetic amino acids are known in the art, and include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbornane)-carboxylic acid, α,γ-diaminobutyric acid, α,β-diaminopropionic acid, homophenylalanine, and α-tert-butylglycine.

The polypeptides, proteins, or CARs of aspects of the invention (including functional portions and functional variants thereof) can be obtained by methods known in the art. The polypeptides, proteins, or CARs may be made by any suitable method of making polypeptides or proteins. Suitable methods of de novo synthesizing polypeptides and proteins are known in the art. Also, polypeptides and proteins can be recombinantly produced using nucleic acids and standard recombinant methods. Alternatively, the polypeptides, proteins, or CARs described herein (including functional portions and functional variants thereof) can be commercially synthesized by any of a variety of commercial entities.

An aspect of the invention provides a nucleic acid comprising a nucleotide sequence encoding any of the polypeptides, proteins, CARs, anti-mesothelin binding moieties, conjugates, or functional portions or functional variants thereof described herein.

“Nucleic acid,” as used herein, includes “polynucleotide,” “oligonucleotide,” and “nucleic acid molecule,” and generally means a polymer of DNA or RNA, which can be single-stranded or double-stranded, synthesized or obtained (e.g., isolated and/or purified) from natural sources, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide. In some aspects, the nucleic acid does not comprise any insertions, deletions, inversions, and/or substitutions. However, it may be suitable in some instances, as discussed herein, for the nucleic acid to comprise one or more insertions, deletions, inversions, and/or substitutions. In some aspects, the nucleic acid may encode additional amino acid sequences that do not affect the function of the polypeptide, protein, or CAR and which may or may not be translated upon expression of the nucleic acid by a host cell (e.g., AAA). In an aspect of the invention, the nucleic acid is complementary DNA (cDNA). In an aspect of the invention, the nucleic acid comprises a codon-optimized nucleotide sequence.

The nucleic acids of an aspect of the invention may be recombinant. As used herein, the term “recombinant” refers to (i) molecules that are constructed outside living cells by joining natural or synthetic nucleic acid segments to nucleic acid molecules that can replicate in a living cell, or (ii) molecules that result from the replication of those described in (i) above. For purposes herein, the replication can be in vitro replication or in vivo replication.

A recombinant nucleic acid may be one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, e.g., by genetic engineering techniques, such as those described in Green and Sambrook, Molecular Cloning: A Laboratory Manual, Cold Spring Harbor Laboratory Press, 4^{th} Ed. (2012). The nucleic acids can be constructed based on chemical synthesis and/or enzymatic ligation reactions using procedures known in the art. See, for example, Green et al., supra. For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of modified nucleotides that can be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxy hydroxymethyl) uracil, 5-carboxy methylaminomethyl-2-thiouridine, 5-carboxy methylaminomethyluracil, dihydrouracil, beta-D-galactosylqueosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, N⁶-substituted adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N⁶-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine. Alternatively, one or more of the nucleic acids of the invention can be synthesized by any of a variety of commercial entities.

In an aspect of the invention, the nucleic acid comprising a nucleotide sequence encoding any of the polypeptides, proteins, and CARs described herein may further comprise a nucleotide sequence encoding truncated human epidermal growth factor receptor (huEGFRt). Transduction of T cells with a nucleic acid construct (such as a lentivirus vector) encoding both huEGFRt and any of the polypeptides, proteins, and CARs described herein may allow for selection of transduced T cells using labelled EGFR monoclonal antibody cetuximab (ERBITUX™). For example, cetuximab can be labeled with biotin and transduced T cells can be selected using anti-biotin magnetic beads, which are commercially available (such as from Miltenyi Biotec). Co-expression of huEGFRt may also allow for in vivo tracking of adoptively transferred CAR-expressing T cells. Furthermore, binding of cetuximab to T cells expressing huEGFRt induces cytotoxicity of effector cells, thereby providing a mechanism to eliminate transduced T cells in vivo (Wang et al, Blood 118(5): 1255-1263, 2011), such as at the conclusion of therapy. Nucleic acids comprising a nucleotide sequence encoding huEGFRt are disclosed in U.S. Patent Application Publication No. 2021/0371542, which is incorporated by reference herein in its entirety.

It is also contemplated that the inventive polypeptides, proteins, and CARs may be useful for preparing bicistronic nucleic acid constructs encoding first and second CARs. In this regard, an aspect of the invention provides a nucleic acid comprising a nucleotide sequence encoding a CAR: (a) a first CAR, comprising a first antigen binding domain, a first TM domain, and a first intracellular T cell signaling domain: (b) a second CAR comprising a second antigen binding domain, a second TM domain, and a second intracellular T cell signaling domain: and (c) a cleavage sequence, wherein the cleavage sequence is positioned between the first and second CARs, and wherein the second CAR specifically binds to a human mesothelin epitope other than human mesothelin₅₈₂₋₅₉₈ (IPNGYLVLDLSMQEALS) (SEQ ID NO: 1).

Cleavage sequences are known in the art and include, but are not limited to, 2A self-cleaving peptides. Examples of 2A self-cleaving peptides include those derived from P2A, E2A, F2A, and T2A.

The first CAR has antigen specificity for human mesothelin₅₈₂₋₅₉₈ (IPNGYLVLDLSMQEALS) (SEQ ID NO: 1) and comprises an antigen binding domain as described herein with respect to other aspects of the invention.

The second CAR may comprise the antigen binding domain of any anti-mesothelin antibody known in the art having antigen specificity for a human mesothelin epitope other than human mesothelin₅₈₂₋₅₉₈ (IPNGYLVLDLSMQEALS) (SEQ ID NO: 1), as described herein with respect to other aspects of the invention. In an aspect of the invention, the antigen binding domain of the second CAR comprises the antigen binding domain of mAb YP218 or humanized mAb YP218, as described herein with respect to other aspects of the invention.

The first and second CARs may otherwise be as described herein with respect to other aspects of the invention.

In an aspect of the invention, the nucleic acid comprising a nucleotide sequence encoding first and second CARs encodes the amino acid sequence of SEQ ID NO: 93.

It is also contemplated that the inventive polypeptides, proteins, and CARs may be useful for preparing bicistronic nucleic acid constructs encoding a CAR and any of the polypeptides, proteins or anti-mesothelin binding moieties described herein with respect to other aspects of the invention. In this regard, an aspect of the invention provides a nucleic acid comprising a nucleotide sequence encoding: (a) any of the polypeptides, proteins or anti-mesothelin binding moieties described herein with respect to other aspects of the invention: (b) a CAR comprising an antigen binding domain, a TM domain, and an intracellular T cell signaling domain: and (c) a cleavage sequence, wherein the cleavage sequence is positioned between the polypeptide and the CAR, and wherein the CAR specifically binds to a human mesothelin epitope other than human mesothelin₅₈₂₋₅₉₈ (IPNGYLVLDLSMQEALS) (SEQ ID NO: 1).

The CAR of the bicistronic nucleic acid constructs may comprise the antigen binding domain of any anti-mesothelin antibody known in the art having antigen specificity for a human mesothelin epitope other than human mesothelin₅₈₂₋₅₉₈ (IPNGYLVLDLSMQEALS) (SEQ ID NO: 1), as described herein with respect to other aspects of the invention. In an aspect of the invention, the antigen binding domain of the second CAR comprises the antigen binding domain of mAb YP218 or humanized mAb YP218, as described herein with respect to other aspects of the invention. The CAR may otherwise be as described herein with respect to other aspects of the invention.

In an aspect of the invention, the bicistronic nucleic acid construct encoding a CAR and any of the polypeptides, proteins or anti-mesothelin binding moieties described herein comprises a nucleotide sequence encoding the amino acid sequence of SEQ ID NO: 96.

Another aspect of the invention provides a polypeptide encoded by any of the nucleic acids described herein.

In an aspect, the nucleic acids of the invention can be incorporated into a recombinant expression vector. In this regard, an aspect of the invention provides recombinant expression vectors comprising any of the nucleic acids of the invention. For purposes herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors of the invention are not naturally-occurring as a whole. However, parts of the vectors can be naturally-occurring. The inventive recombinant expression vectors can comprise any type of nucleotides, including, but not limited to DNA and RNA, which can be single-stranded or double-stranded, synthesized or obtained in part from natural sources, and which can contain natural, non-natural or altered nucleotides. The recombinant expression vectors can comprise naturally-occurring or non-naturally-occurring internucleotide linkages, or both types of linkages. Preferably, the non-naturally occurring or altered nucleotides or internucleotide linkages do not hinder the transcription or replication of the vector.

In an aspect, the recombinant expression vector of the invention can be any suitable recombinant expression vector, and can be used to transform or transfect any suitable host cell. Suitable vectors include those designed for propagation and expansion or for expression or both, such as plasmids and viruses. The recombinant expression vector may be a transposon or a viral vector, e.g., a retroviral vector or a lentiviral vector.

In an aspect, the recombinant expression vectors of the invention can be prepared using standard recombinant DNA techniques described in, for example, Green, supra. Constructs of expression vectors, which are circular or linear, can be prepared to contain a replication system functional in a prokaryotic or eukaryotic host cell.

The recombinant expression vector may comprise regulatory sequences, such as transcription and translation initiation and termination codons, which are specific to the type of host cell (e.g., bacterium, fungus, plant, or animal) into which the vector is to be introduced, as appropriate, and taking into consideration whether the vector is DNA- or RNA-based. The recombinant expression vector may comprise restriction sites to facilitate cloning.

The recombinant expression vector can include one or more marker genes, which allow for selection of transformed or transfected host cells. Marker genes include biocide resistance, e.g., resistance to antibiotics, heavy metals, etc., complementation in an auxotrophic host to provide prototrophy, and the like. Suitable marker genes for the inventive expression vectors include, for instance, neomycin/G418 resistance genes, hygromycin resistance genes, histidinol resistance genes, tetracycline resistance genes, and ampicillin resistance genes.

The recombinant expression vector can comprise a native or nonnative promoter operably linked to the nucleotide sequence encoding the polypeptides, proteins, CARs, anti-mesothelin binding moieties, conjugates, or functional portions or functional variants thereof. The selection of promoters, e.g., strong, weak, inducible, tissue-specific and developmental-specific, is within the ordinary skill of the artisan. Similarly, the combining of a nucleotide sequence with a promoter is also within the ordinary skill of the artisan. The promoter can be a non-viral promoter or a viral promoter, e.g., a cytomegalovirus (CMV) promoter, an SV40 promoter, an RSV promoter, or a promoter found in the long-terminal repeat of the murine stem cell virus.

The inventive recombinant expression vectors can be designed for either transient expression, for stable expression, or for both. Also, the recombinant expression vectors can be made for constitutive expression or for inducible expression.

An aspect of the invention further provides a host cell comprising any of the recombinant expression vectors described herein. As used herein, the term “host cell” refers to any type of cell that can contain the inventive recombinant expression vector. The host cell can be a eukaryotic cell, e.g., plant, animal, fungi, or algae, or can be a prokaryotic cell, e.g., bacteria or protozoa. The host cell can be a cultured cell or a primary cell, i.e., isolated directly from an organism, e.g., a human. The host cell can be an adherent cell or a suspended cell, i.e., a cell that grows in suspension. Suitable host cells are known in the art and include, for instance, DH5α E. coli cells, Chinese hamster ovarian cells, monkey VERO cells, COS cells, HEK293 cells, and the like. For purposes of amplifying or replicating the recombinant expression vector, the host cell may be a prokaryotic cell, e.g., a DH5α cell. For purposes of producing a recombinant polypeptide, protein, CAR, anti-mesothelin binding moiety, conjugate, or functional portion or functional variant thereof, the host cell may be a mammalian cell. The host cell may be a human cell. While the host cell can be of any cell type, can originate from any type of tissue, and can be of any developmental stage, the host cell may be a peripheral blood lymphocyte (PBL) or a peripheral blood mononuclear cell (PBMC). The host cell may be a B cell, a T cell, or an NK cell.

For purposes herein, the T cell can be any T cell, such as a cultured T cell, e.g., a primary T cell, or a T cell from a cultured T cell line, e.g., Jurkat, SupT1, etc., or a T cell obtained from a mammal. If obtained from a mammal, the T cell can be obtained from numerous sources, including but not limited to blood, bone marrow, lymph node, the thymus, or other tissues or fluids. T cells can also be enriched for or purified. The T cell may be a human T cell. The T cell may be a T cell isolated from a human. The T cell can be any type of T cell and can be of any developmental stage, including but not limited to, CD4⁺/CD8⁺ double positive T cells, CD4⁺ helper T cells, e.g., Th₁ and Th₂ cells, CD8⁺ T cells (e.g., cytotoxic T cells), tumor infiltrating cells, memory T cells, naïve T cells, and the like. The T cell may be a CD8⁺ T cell or a CD4⁺ T cell.

Also provided by an aspect of the invention is a population of cells comprising at least one host cell described herein. The population of cells can be a heterogeneous population comprising the host cell comprising any of the recombinant expression vectors described, in addition to at least one other cell, e.g., a host cell (e.g., a T cell), which does not comprise any of the recombinant expression vectors, or a cell other than a T cell, e.g., a B cell, a macrophage, a neutrophil, an erythrocyte, a hepatocyte, an endothelial cell, an epithelial cell, a muscle cell, a brain cell, etc. Alternatively, the population of cells can be a substantially homogeneous population, in which the population comprises mainly host cells (e.g., consisting essentially of) comprising the recombinant expression vector. The population also can be a clonal population of cells, in which all cells of the population are clones of a single host cell comprising a recombinant expression vector, such that all cells of the population comprise the recombinant expression vector. In one aspect of the invention, the population of cells is a clonal population comprising host cells comprising a recombinant expression vector as described herein.

The polypeptides, proteins, anti-mesothelin binding moieties, CARs (including functional portions and variants thereof), nucleic acids, recombinant expression vectors, host cells (including populations thereof), and conjugates, all of which are collectively referred to as “inventive anti-mesothelin materials” hereinafter, can be isolated and/or purified. The term “isolated” as used herein means having been removed from its natural environment. The term “purified” or “isolated” does not require absolute purity or isolation: rather, it is intended as a relative term. Thus, for example, a purified (or isolated) host cell preparation is one in which the host cell is more pure than cells in their natural environment within the body. Such host cells may be produced, for example, by standard purification techniques. In some aspects, a preparation of a host cell is purified such that the host cell represents at least about 50%, for example, at least about 70%, of the total cell content of the preparation. For example, the purity can be at least about 50%, can be greater than about 60%, about 70% or about 80%, or can be about 100%.

The inventive anti-mesothelin materials can be formulated into a composition, such as a pharmaceutical composition. In this regard, an aspect of the invention provides a pharmaceutical composition comprising any of the inventive anti-mesothelin materials described herein and a pharmaceutically acceptable carrier. The inventive pharmaceutical compositions containing any of the inventive anti-mesothelin materials can comprise more than one inventive anti-mesothelin material, e.g., a conjugate and a nucleic acid, or two or more different conjugates Alternatively, the pharmaceutical composition can comprise an inventive anti-mesothelin material in combination with other pharmaceutically active agents or drugs, such as chemotherapeutic agents, e.g., asparaginase, bortezomib (e.g., VELCADE bortezomib), busulfan, carboplatin, cisplatin, daunorubicin, dexamethasone, doxorubicin, fluorouracil, gemcitabine, hydroxyurea, lenalidomide, melphalan, methotrexate, paclitaxel (e.g., ABRAXANE paclitaxel), rituximab, vinblastine, vincristine, etc. Preferably, the other pharmaceutically active agent or drug is melphalan, bortezomib, lenalidomide, dexamethasone, or paclitaxel.

Preferably, the carrier is a pharmaceutically acceptable carrier. With respect to pharmaceutical compositions, the carrier can be any of those conventionally used for the particular inventive anti-mesothelin material under consideration. Methods for preparing administrable compositions are known or apparent to those skilled in the art and are described in more detail in, for example, Remington: The Science and Practice of Pharmacy, 22^{nd} Ed., Pharmaceutical Press (2012). It is preferred that the pharmaceutically acceptable carrier be one which has no detrimental side effects or toxicity under the conditions of use.

The choice of carrier will be determined in part by the particular inventive anti-mesothelin material, as well as by the particular method used to administer the inventive anti-mesothelin material. Accordingly, there are a variety of suitable formulations of the pharmaceutical composition of the invention. Suitable formulations may include any of those for parenteral, subcutaneous, intravenous, intramuscular, intraarterial, intrathecal, intratumoral, or interperitoneal administration. More than one route can be used to administer the inventive anti-mesothelin materials, and in certain instances, a particular route can provide a more immediate and more effective response than another route.

Preferably, inventive anti-mesothelin material is administered by injection, e.g., intravenously. When the inventive anti-mesothelin material is a host cell (or population thereof) expressing the inventive polypeptide, protein, CAR, or anti-mesothelin binding moiety, the pharmaceutically acceptable carrier for the cells for injection may include any isotonic carrier such as, for example, normal saline (about 0.90% w/v of NaCl in water, about 300 mOsm/L NaCl in water, or about 9.0 g NaCl per liter of water), NORMOSOL R electrolyte solution (Abbott, Chicago, IL), PLASMA-LYTE A (Baxter, Deerfield, IL), about 5% dextrose in water, or Ringer's lactate. In an aspect, the pharmaceutically acceptable carrier is supplemented with human serum albumen.

An “effective amount” or “an amount effective to treat” refers to a dose that is adequate to prevent or treat cancer in an individual. Amounts effective for a therapeutic or prophylactic use will depend on, for example, the stage and severity of the cancer being treated, the age, weight, and general state of health of the mammal, and the judgment of the prescribing physician. The size of the dose will also be determined by the active selected, method of administration, timing and frequency of administration, the existence, nature, and extent of any adverse side-effects that might accompany the administration of a particular active, and the desired physiological effect. It will be appreciated by one of skill in the art that various cancer could require prolonged treatment involving multiple administrations, perhaps using the inventive anti-mesothelin materials in each or various rounds of administration. By way of example and not intending to limit the invention, the dose of the inventive anti-mesothelin material can be about 0.001 to about 1000 mg/kg body weight of the mammal being treated/day, from about 0.01 to about 10 mg/kg body weight/day, about 0.01 mg to about 1 mg/kg body weight/day.

For purposes of the invention, the amount or dose of the inventive anti-mesothelin material administered should be sufficient to effect a therapeutic or prophylactic response in the mammal over a reasonable time frame. For example, the dose of the inventive anti-mesothelin material should be sufficient to bind to antigen or detect, treat or prevent cancer in a period of from about 2 hours or longer, e.g., about 12 to about 24 or more hours, from the time of administration. In certain aspects, the time period could be even longer. The dose will be determined by the efficacy of the particular inventive anti-mesothelin material and the condition of the animal (e.g., human), as well as the body weight of the animal (e.g., human) to be treated.

For purposes of the invention, an assay, which comprises, for example, comparing the extent to which target cells are killed upon administration of a given dose of the inventive anti-mesothelin material to a mammal, among a set of mammals of which is each given a different dose of the inventive anti-mesothelin material, could be used to determine a starting dose to be administered to a mammal. The extent to which target cells are killed upon administration of a certain dose can be assayed by methods known in the art.

It is contemplated that the inventive anti-mesothelin materials and pharmaceutical compositions can be used in methods of treating or preventing cancer in a mammal. Without being bound to a particular theory or mechanism, the inventive anti-mesothelin materials have biological activity, e.g., ability to recognize antigen, e.g., mesothelin, such that the anti-mesothelin material, can direct an effector molecule to a target cell or target tissue. In this regard, an aspect of the invention provides a method of treating or preventing cancer, comprising administering to the mammal any of the inventive anti-mesothelin materials or pharmaceutical compositions in an amount effective to treat or prevent cancer in the mammal.

In an aspect of the invention, the method comprises coadministering any of the inventive anti-mesothelin materials and one or more further agents. By “coadministering” is meant administering one or more additional agents and the inventive anti-mesothelin materials sufficiently close in time such that the inventive anti-mesothelin materials can enhance the effect of one or more additional agents, or vice versa.

In an aspect of the invention, the further agent specifically binds to a human mesothelin epitope other than human mesothelin₅₈₂₋₅₉₈ (IPNGYLVLDLSMQEALS) (SEQ ID NO: 1) and inhibits the growth of mesothelin-expressing cells. In this regard, the further agent may comprise the antigen binding domain of an anti-mesothelin antibody known in the art having antigen specificity for a human mesothelin epitope other than human mesothelin₅₈₂₋₅₉₈ (IPNGYLVLDLSMQEALS) (SEQ ID NO: 1), as described herein with respect to other aspects of the invention. In an aspect of the invention, the further agent comprises the antigen binding domain of mAb YP218 or humanized mAb YP218.

Examples of further agents which may be useful when co-administered with any of the inventive anti-mesothelin materials described herein include, but are not limited to, any one or more of the following: a polypeptide, a protein, a conjugate, and a CAR. In an aspect of the invention, the further agent is selected from one or more of the following: an antibody, Fab fragment (Fab), F(ab′)₂ fragment, diabody, triabody, tetrabody, multispecific antibody, scFv, and dsFv. The further agent may be a conjugate comprising (a) an anti-mesothelin binding moiety conjugated or fused to (b) an effector molecule, wherein the effector molecule is a drug, toxin, label, small molecule, or an antibody.

For example, an aspect of the invention provides a method of treating or preventing cancer in a mammal, the method comprising administering to the mammal: (a) any of the inventive polypeptides, proteins, or anti-mesothelin binding moieties described herein with respect to other aspects of the invention: and (b) a further agent that specifically binds to a human mesothelin epitope other than human mesothelin₅₈₂₋₅₉₈ (IPNGYLVLDLSMQEALS) (SEQ ID NO: 1) and inhibits the growth of mesothelin-expressing cells. The method may comprise administering the inventive polypeptide, protein, or anti-mesothelin binding moiety and the further agent sequentially or simultaneously. The method may comprise sequentially administering the inventive polypeptide, protein, or anti-mesothelin binding moiety to the mammal before administering the further agent to the mammal, or vice versa.

An aspect of the invention further comprises lymphodepleting the mammal prior to administering the inventive anti-mesothelin material or pharmaceutical composition. Examples of lymphodepletion include, but may not be limited to, nonmyeloablative lymphodepleting chemotherapy, myeloablative lymphodepleting chemotherapy, total body irradiation, etc.

An aspect of the invention provides a set for treating or preventing cancer in a mammal, wherein the set comprises: (a) any of the inventive polypeptides, proteins, or anti-mesothelin binding moieties described herein with respect to other aspects of the invention, and (b) a further agent that specifically binds to a human mesothelin epitope other than human mesothelin₅₈₂₋₅₉₈ (IPNGYLVLDLSMQEALS) (SEQ ID NO: 1) and inhibits the growth of mesothelin-expressing cells. The further agent that specifically binds to a human mesothelin epitope other than human mesothelin₅₈₂₋₅₉₈ (IPNGYLVLDLSMQEALS) (SEQ ID NO: 1) and inhibits the growth of mesothelin-expressing cells may be as described herein with respect to other aspects of the invention. In an aspect of the invention, the inventive polypeptide, protein, or anti-mesothelin binding moiety and the further agent are to be administered sequentially or simultaneously. In an aspect, the inventive polypeptide, protein, or anti-mesothelin binding moiety is to be sequentially administered to the mammal before the further agent is administered to the mammal, or vice versa.

The mammal referred to herein can be any mammal. As used herein, the term “mammal” refers to any mammal, including, but not limited to, mammals of the order Rodentia, such as mice and hamsters, and mammals of the order Lagomorpha, such as rabbits. The mammals may be from the order Carnivora, including Felines (cats) and Canines (dogs). The mammals may be from the order Artiodactyla, including Bovines (cows) and Swines (pigs) or of the order Perssodactyla, including Equines (horses). The mammals may be of the order Primates, Ceboids, or Simoids (monkeys) or of the order Anthropoids (humans and apes). Preferably, the mammal is a human.

With respect to the inventive methods, the cancer can be any cancer, including any of acute lymphocytic cancer, acute myeloid leukemia, rhabdomyosarcoma, bladder cancer (e.g., bladder carcinoma), bone cancer, brain cancer (e.g., medulloblastoma, neuroblastoma, and glioblastoma), breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the ovary, vulva, endometrium, or chronic lymphocytic leukemia, chronic myeloid cancer, colon cancer, Ewing's sarcoma, esophageal cancer, cervical cancer, fibrosarcoma, gastrointestinal carcinoid tumor, head and neck cancer (e.g., head and neck squamous cell carcinoma), Hodgkin lymphoma, hypopharynx cancer, kidney cancer, larynx cancer, leukemia, liquid tumors, liver cancer, lung cancer (e.g., non-small cell lung carcinoma, lung adenocarcinoma), lymphoma, malignant mesothelioma, mastocytoma, melanoma, multiple myeloma, nasopharynx cancer, neuroblastoma, non-Hodgkin lymphoma, B-chronic lymphocytic leukemia, hairy cell leukemia, acute lymphocytic leukemia (ALL), and Burkitt's lymphoma, ovarian cancer, pancreatic cancer, gastric cancer, peritoneum, omentum, and mesentery cancer, pharynx cancer, prostate cancer, rectal cancer, renal cancer, skin cancer, small intestine cancer, soft tissue cancer, solid tumors, stomach cancer, testicular cancer, thyroid cancer, synovial cancer, and ureter cancer. Preferably, the cancer is ovarian cancer, endometrial cancer, cervical cancer, colonic cancer, pancreatic cancer, lung cancer (e.g., non-small cell lung carcinoma, lung adenocarcinoma), esophageal cancer, gastric cancer, synovial sarcoma, and mesothelioma. In an aspect, the cancer is characterized by the expression or overexpression of mesothelin.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of treatment or prevention of cancer in a mammal. Furthermore, the treatment or prevention provided by the inventive methods can include treatment or prevention of one or more conditions or symptoms of the cancer being treated or prevented. Also, for purposes herein, “prevention” can encompass delaying the onset of the cancer, or a symptom or condition thereof.

Another aspect of the invention provides a method of reducing mesothelin shed from cell membranes, the method comprising administering to cells any of the inventive anti-mesothelin materials or pharmaceutical compositions described herein, in an amount effective to reduce mesothelin shed from the cell membranes of the cells.

As used herein, “reducing mesothelin shed from cell membranes” means a reduction in the quantity of mesothelin protein, or portion thereof, removed from a cell following administration of one or more of the inventive anti-mesothelin materials compared to the quantity that would have been removed from the cell without the administration of the inventive anti-mesothelin material to the cells.

Another aspect of the invention provides a method of detecting the presence of cancer in a mammal comprising: (a) contacting a sample comprising one or more cells from the mammal with any of the inventive anti-mesothelin materials described herein, thereby forming a complex, (b) and detecting the complex, wherein detection of the complex is indicative of the presence of cancer in the mammal.

The sample may be obtained by any suitable method, e.g., biopsy or necropsy. A biopsy is the removal of tissue and/or cells from a mammal. Such removal may be to collect tissue and/or cells from the mammal in order to perform experimentation on the removed tissue and/or cells. This experimentation may include experiments to determine if the mammal has and/or is suffering from cancer.

With respect to an aspect of the inventive method of detecting the presence of cancer in a mammal, the sample comprising cells of the mammal can be a sample comprising whole cells, lysates thereof, or a fraction of the whole cell lysates, e.g., a nuclear or cytoplasmic fraction, a whole protein fraction, or a nucleic acid fraction. If the sample comprises whole cells, the cells can be any cells of the mammal, e.g., the cells of any organ or tissue, including blood cells or endothelial cells.

For purposes of the inventive detecting method, the contacting can take place in vitro or in vivo with respect to the mammal. Preferably, the contacting is in vitro.

The following examples further illustrate the invention but, of course, should not be construed as in any way limiting its scope.

### EXAMPLE 1

This example demonstrates that mAb 15B6 does not bind to mesothelin shed by OVCAR-8 cells.

The amino acid sequences of the VH and VL of mAb 15B6 are shown in Table 1. In Table 1, the CDRs are underlined, and the framework regions are shown in italics.

TABLE 1

| Amino acid Sequence | mAb Name | Region |
|---|---|---|
| EVQLQQSGPVLVKPGASVKISCKASGYSFT | 15B6 | Vh |
| GYYMHWVRQSLVKRLEWIGRINPYTGVPSY | (IgG2b) | |
| KHNFKDKASLTVDKSSSTAYMELHSLTSED | | |
| SAVYYCARELGGYWGQGTTLTVSS | | |
| (SEQ ID NO: 44) | | |
| QAVVTQESALTTSPGETVTLTCRSSTGAVT | 15B6 | Vl |
| TGNYPNWVQEKPDHLFTGLIAGTNNRAPGV | (IgG2b) | |
| PARFSGSLIGDKAALTITGAQTEDEAIYFC | | |
| ALWFSSHWVFGGGTKLTVL | | |
| (SEQ ID NO: 45) | | |

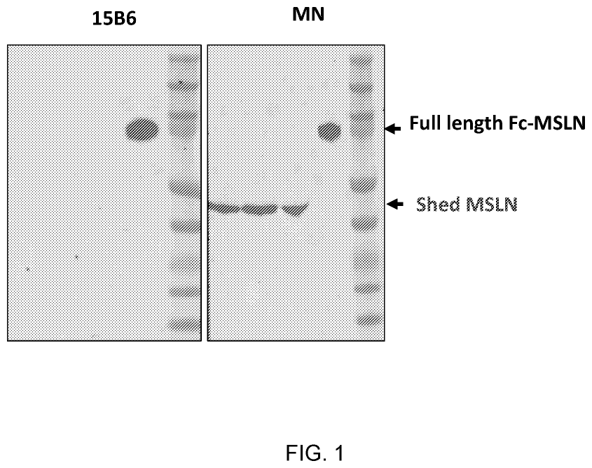

A Western blot assay was carried out to test the ability of mAb 15B6 or control mAb MN to bind to full length Fc-mesothelin and shed mesothelin, which had been shed by OVCAR-8 cells. Equal amounts of OVCAR-8 cell growth media were collected. The proteins were resolved by SDS gel electrophoresis. The bands were detected by Western blot using the antibodies MN and/or 15B6. Full length mesothelin attached to an Fc protein was used as positive control and reacts with both antibodies. The results are shown in FIG. 1.

As shown in FIG. 1, control mAb MN bound to full length Fc-mesothelin and shed mesothelin, while mAb 15B6 bound only to full length Fc-mesothelin and not to shed mesothelin.

### EXAMPLE 2

This example demonstrates that mAb 15B6 binds to mesothelin positive cells but not to mesothelin negative cells.

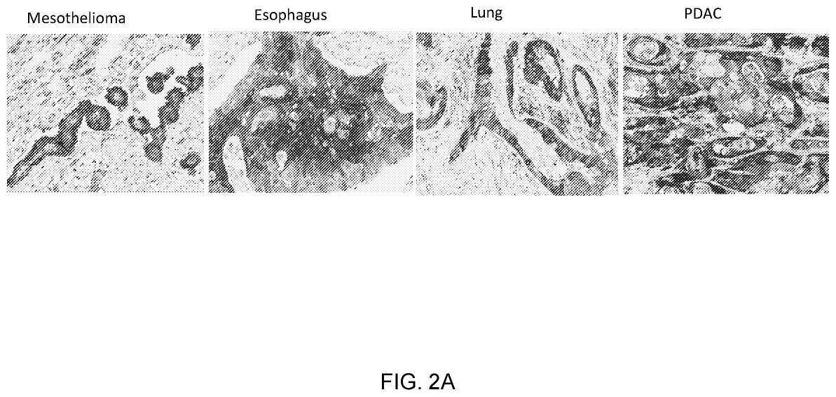

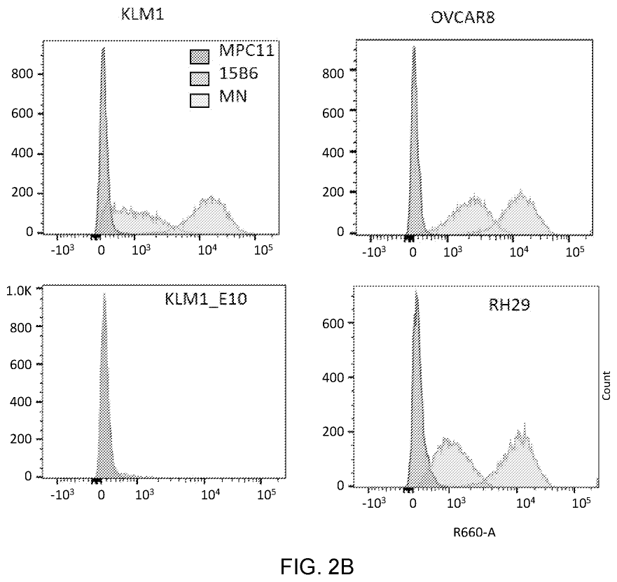

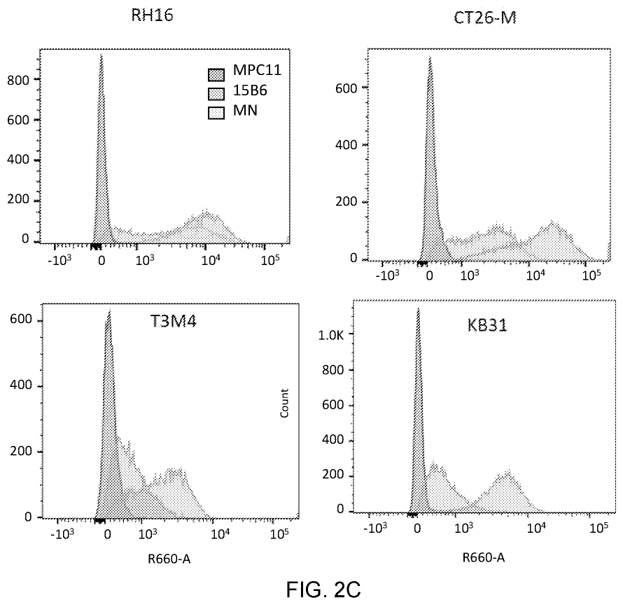

Immunohistochemistry (IHC) assays were carried out to test the ability of mAb 15B6 to bind to mesothelin-expressing tumor samples of mesothelioma, esophagus, lung, and pancreatic ductal adenocarcinoma (PDAC). Formalin-fixed tumor samples were exposed to mAb 15B6. Binding was detected by an anti-mouse IgG labelled with peroxidase. The results are shown in FIG. 2A. As shown in FIG. 2A, mAb 15B6 bound to the mesothelin-expressing tumor samples of all of mesothelioma, esophagus, lung, and PDAC.

Flow cytometry studies were carried out to test the ability of mAb 15B6 to bind to mesothelin-expressing tumor cell lines KLM1, OVCAR8, RH16, CT26-M, RH29, T3M4,and KB31 or to control tumor cell line KLM1_E10, which does not express mesothelin. The tumor cells were trypsinized from monolayer culture and incubated with either MN antibody conjugated to ALEXA FLUOR 647 fluorescent dye (Thermo Fisher Scientific, Waltham, MA) or 15B6 antibody conjugated to ALEXA FLUOR 647 fluorescent dye on ice for one hour. Cells were analyzed by flow cytometry after washing unbound antibodies. The results are shown in FIGS. 2B-2C. As shown in FIGS. 2B-2C, mAb 15B6 bound to all of the mesothelin-expressing tumor cell lines, but not to the mesothelin-negative tumor cell line, KLM1_E10.

Mesothelin is highly expressed by cancer cells and is not expressed by essential normal tissues. IHC assays were carried out to test the ability of mAb 15B6 to bind to normal tissues, including stomach, kidney, heart, liver, spleen, lung, brain, colon, testis, and ovary. The results showed that mAb 15B6 did not bind to any of the normal tissues.

### EXAMPLE 3

This example demonstrates that mAb 15B6 inhibits the shedding of mesothelin from cancer cell lines.

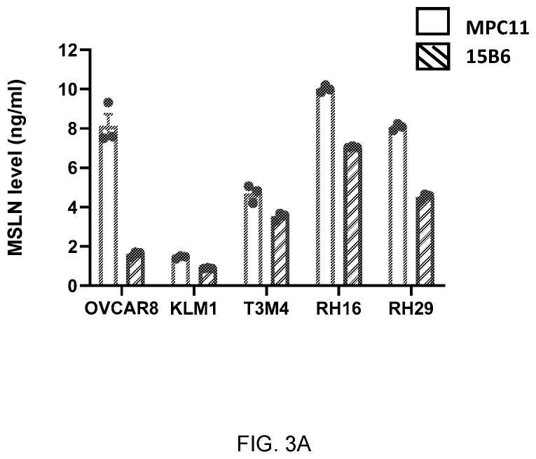

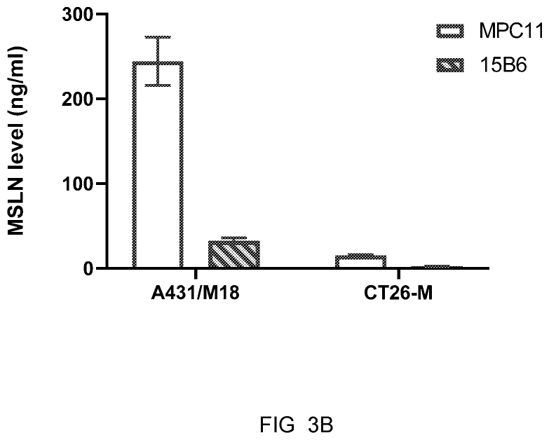

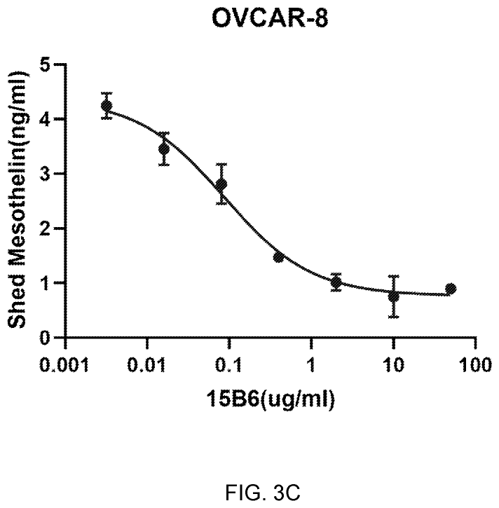

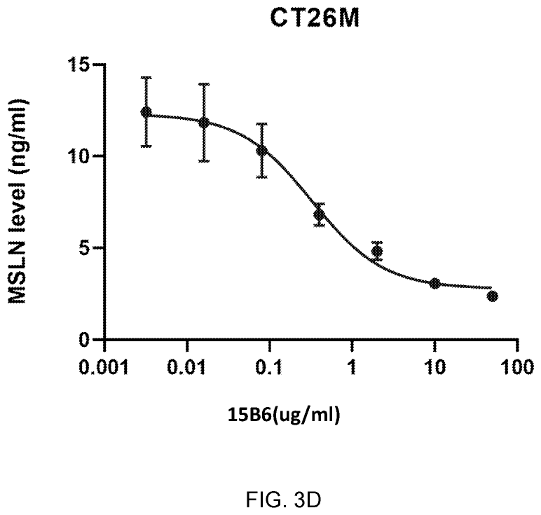

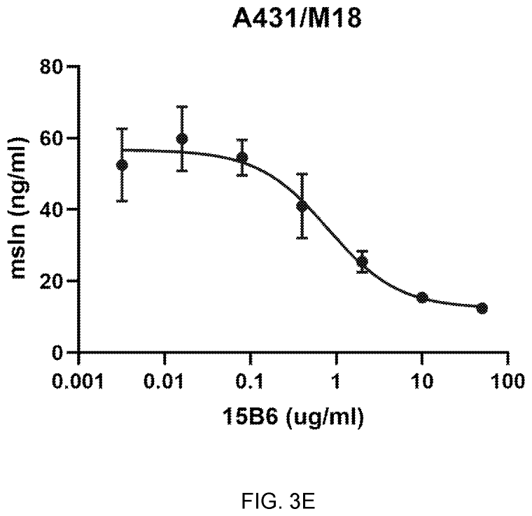

Assays were carried out to test the ability of mAb 15B6 or control mAb MPC11 to inhibit the shedding of mesothelin from cancer cell lines. Cell lines were incubated with mAb 15B6 or mAb MPC11 for two (2) days. The amount of shed mesothelin in the medium was detected by enzyme-linked immunoassay (ELISA). The results are shown in FIGS. 3A-3B. As shown in FIGS. 3A-3B, the amount of shed mesothelin detected in the medium was lower for those cancer lines incubated with mAb 15B6 as compared to those incubated with mAb MPC11.

Assays were carried out to test the ability of mAb 15B6 to inhibit the shedding of mesothelin from cancer cell lines. Cell lines were incubated with various concentrations of mAb 15B6 for two (2) days. The amount of shed mesothelin in the medium was detected by ELISA. The results are shown in FIGS. 3C-3E. These results show dose-dependent inhibition of mesothelin shedding in three different cell lines.

### EXAMPLE 4

This example demonstrates that T cells expressing a CAR comprising the antigen binding domain of mAb 15B6 are more cytotoxic toward cancer cells as compared to T cells expressing a CAR comprising the antigen binding domain of control mAb SS1.

CAR-T cells (effector cells) were produced by transducing healthy human donor PBMC with a retroviral vector encoding a CAR comprising the antigen binding domain of mAb 15B6 (CAR Construct No. 1 of Table 3) (15B6 CAR-T cells) or a CAR comprising the antigen binding domain of control anti-mesothelin mAb SS1 (SS1 CAR-T cells). Each CAR further comprised a CD8 hinge domain, a CD8 TM domain, a 4-1BB intracellular signaling domain, and a CD3 zeta intracellular signaling domain. Mock-transfected CAR-T cells (no scFv) was used as a control. The mAb SS1 binds at the amino terminus of mesothelin.

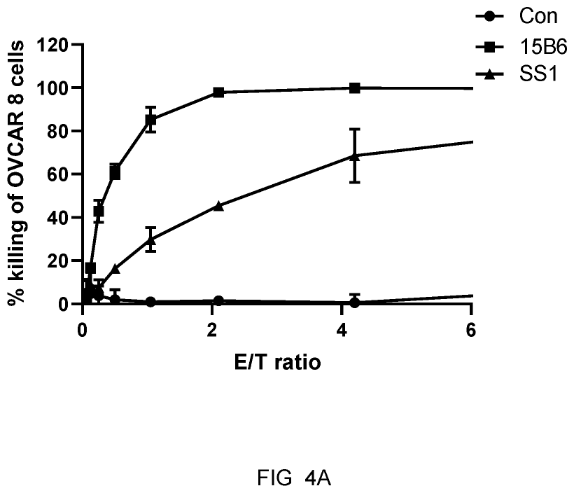

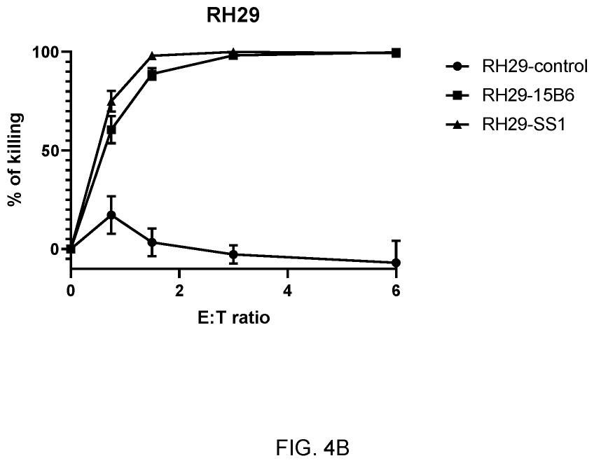

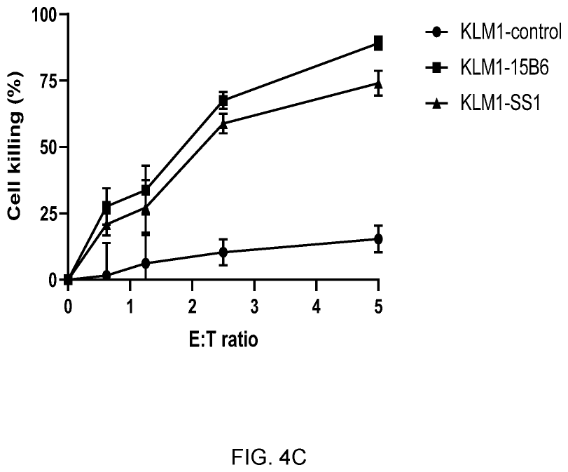

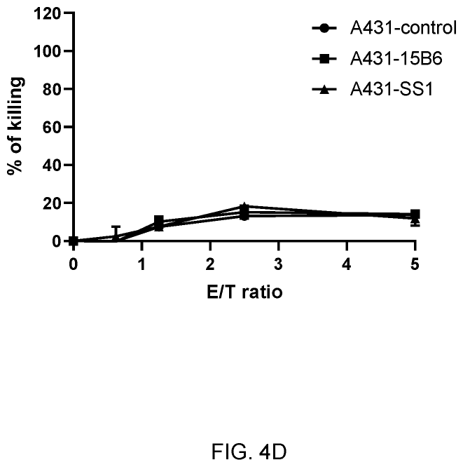

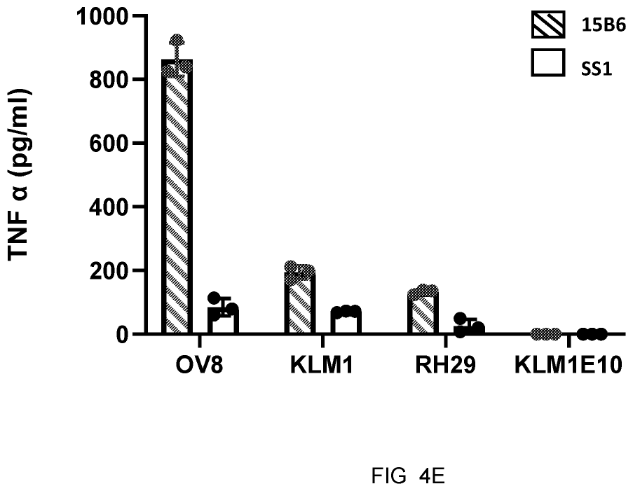

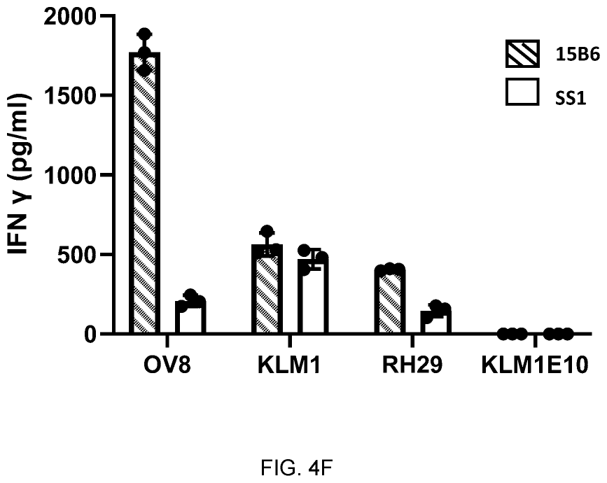

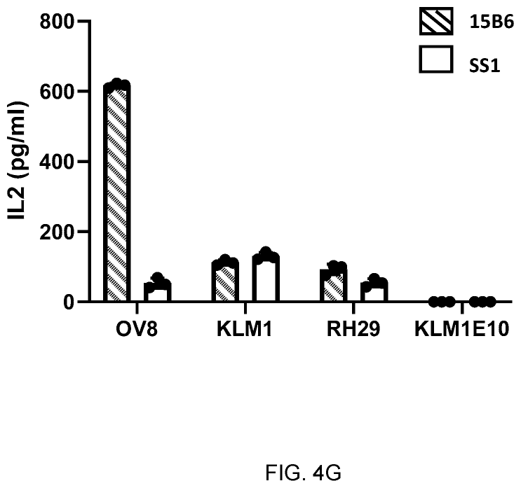

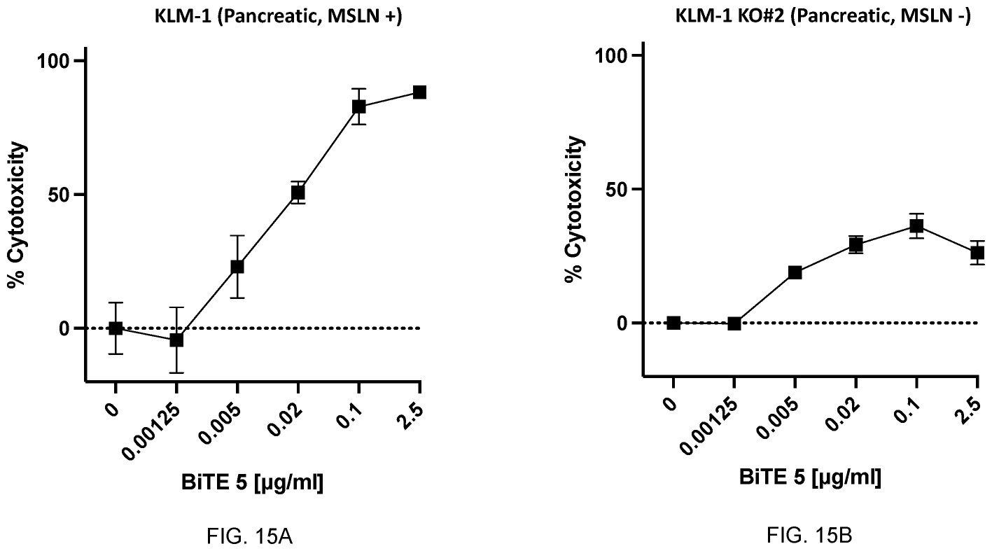

CAR-T cells were independently co-cultured with cancer cell lines OVCAR8, RH29, KLM1, and A431 (target cells) at various effector to target ratios. The percentage of target cells killed following co-culture was determined. The results are shown in FIGS. 4A-4D. As shown in FIGS. 4A-4D, 15B6 CAR-T cells were more active killing target cancer cells as compared to SS1 CAR-T cells.

The effector cells described for FIGS. 4A-4D were co-cultured with target cell OVCAR8, RH29, KLM1, or control (mesothelin-negative) cancer cell line KLM1E10. The concentration of TNF-alpha, IFN-gamma, and IL-2 secreted following the co-culture was measured. The results are shown in FIGS. 4E-4G. As shown in FIGS. 4E-4G, 15B6 CAR-T cells secreted more TNF-alpha, IFN-gamma, and IL-2 following co-culture with OVCAR8 as compared to SS1 CAR-T cells.

### EXAMPLE 5

This example demonstrates that the cytotoxic activity of 15B6 CAR-T cells is not blocked by shed mesothelin.

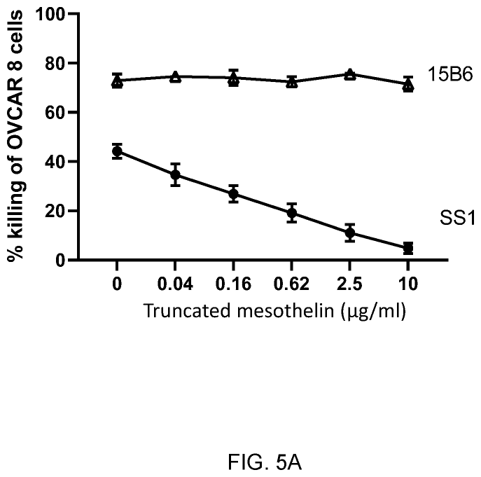

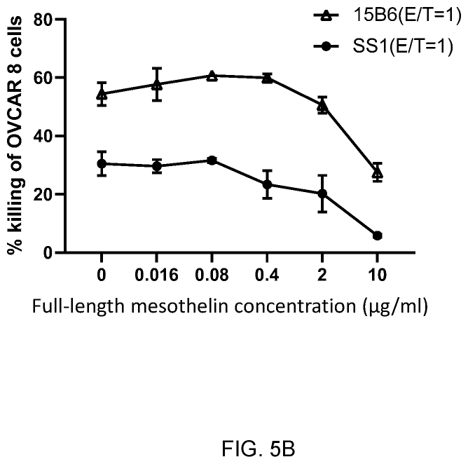

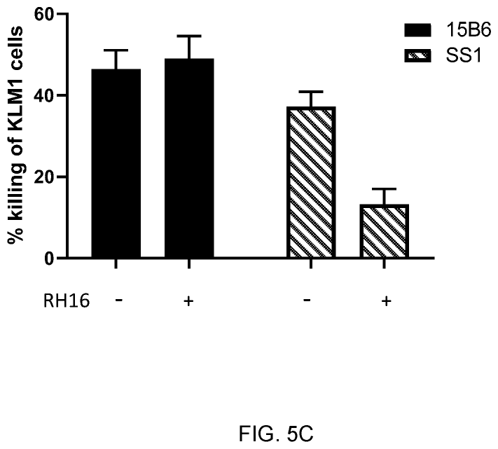

The effector cells described for Example 4 were co-cultured with target OVCAR8 cells in the presence of various concentrations of truncated mesothelin (amino acid residues 295-585) or full-length mesothelin (295-599) at an effector to target ratio of 1:1. The percentage of OVCAR8 cells killed following co-culture was determined. The results are shown in FIGS. 5A-5B.

As shown in FIG. 5A, the cytotoxic activity of SS1 CAR-T cells was blocked by truncated mesothelin corresponding to the size of mesothelin shed by OVCAR-8 cells. In contrast, the cytotoxic activity of 15B6 CAR-T cells was not blocked because it does not bind to shed mesothelin (FIG. 5A). Full length mesothelin (295-599) blocked the cytotoxic activity of both SS1 CAR-T cells and 15B6 CAR-T cells (FIG. 5B).

The effector cells described for Example 4 were co-cultured with target KLM1 cells in the presence or absence of ascites from mesothelioma patient RH16. The percentage of KLM1 cells killed following co-culture was determined. The results are shown in FIG. 5C. As shown in FIG. 5C, ascites from the mesothelioma patient blocked SS1 CAR-T cells but not 15B6 CAR-T cells.

### EXAMPLE 6

This example demonstrates that 15B6 CAR T cells are more active than SS1 CAR-T cells in reducing OVCAR-8 tumor burden in mice.

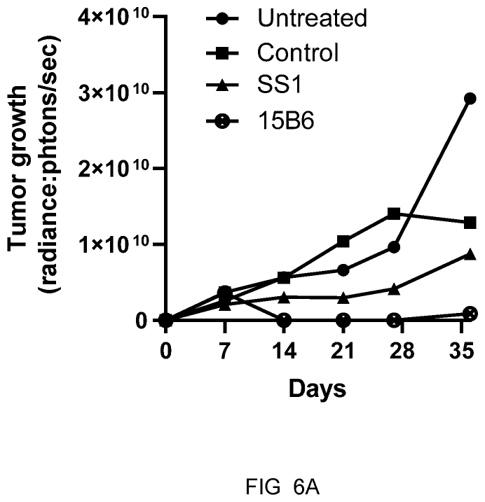

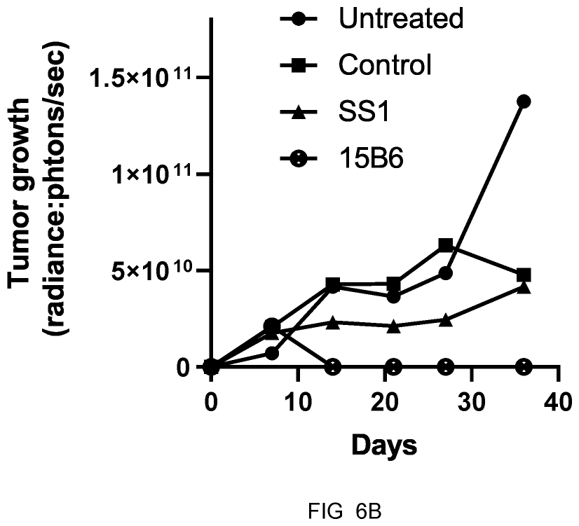

Mice were injected on day 0 with OVCAR-8 cells transfected with luciferase. On Day 8, mice were administered 1×10⁷ (i) SS1 CAR-T cells, (ii) 15B6 CAR-T cells, or (iii) control CAR T cell intravenously (IV) (dorsal or ventral) or were left untreated. The SS1 CAR-T cells and the 15B6 CAR-T cells were as described for Example 4. Luciferin was injected into the mice, and tumor burden was measured in terms of bioluminescent signals per each mouse as radiance: photons/second with bioluminescent imaging from day 0 to day 35. The results are shown in FIGS. 6A-6B. The 15B6 CAR T cells were more active in reducing OVCAR-8 tumor burden as compared to SS1 CAR-T cells.

### EXAMPLE 7

This example demonstrates the ability of various mAb 15B6-based CAR constructs to be expressed by T cells.

CAR-T vectors encoding CARs comprising any one of six different mAb 15B6-based single chain constructs (Fv) were prepared. The amino acid sequences of the six mAb 15B6-based Fv constructs used in the CARs are shown in Table 2. In the amino acid sequences shown in Table 2, cysteine-substituted amino acid residues (if any) are in bold font, the CDRs are underlined, and the framework regions are in italics.

Each CAR further comprised a CD8 hinge domain, a CD8 TM domain, a 4-1BB intracellular signaling domain, and a CD3 zeta intracellular signaling domain. The full-length amino acid sequences of the CARs of Construct Nos. 1 and 2 are shown in Table 3. In Table 3, the Fv of the CAR is in bold font, and the TM and T-cell activation domain are italicized.

293T cells were independently transduced with the CAR-T vectors. Expression of each Fv by the 293T cells was tested. The CAR-T vectors were transfected into 293T cells. After two days of culture, Fc-mesothelin was bound to the cells. The bound Fc protein was detected by a secondary anti-Fc-antibody conjugated to a fluorescent dye using flow cytometry. The results are shown in Table 4. As shown in Table 4, only the CARs comprising the Fv of Construct Nos. 1 or 2 were expressed by 293T cells.

TABLE 2

| Fv | Fv | | |
|---|---|---|---|
| Construct | Construct | | |
| No. | Name | Fv Description | Fv Sequence |
| 1 | 15B6 scFv | scFv with the mAb 15B6 | QAVVTQESALTTSPGETVTLTCRSS |
| | (Vl-lin-Vh) | VL at the N-terminus, the | TGAVTTGNYPNWVQEKPDHLFTGLI |
| | | mAb 15B6 VH at the C- | AGTNNRAPGVPARFSGSLIGDKAAL |
| | | terminus, and a [G₄S]₃ | TITGAQTEDEAIYFCALWFSSHWVF |
| | | linker positioned in | GGGTKLTVLGGGGSGGGGSGGGGSE |
| | | between the Vl and the | VQLQQSGPVLVKPGASVKISCKASG |
| | | Vh | YSFTGYYMHWVRQSLVKRLEWIGRI |
| | | | NPYTGVPSYKHNFKDKASLTVDKSS |
| | | | STAYMELHSLTSEDSAVYYCARELG |
| | | | GYWGQGTTLTVSS |
| | | | (SEQ ID NO: 50) |
| 2 | 15B6 scFv | scFv with the mAb 15B6 | MEVQLQQSGPVLVKPGASVKISCKA |
| | (Vh-lin-Vl) | Vh at the N-terminus, the | SGYSFTGYYMHWVRQSLVKRLEWIG |
| | | mAb 15B6 Vl at the C- | RINPYTGVPSYKHNFKDKASLTVDK |
| | | terminus, and a [G₄S]₃ | SSSTAYMELHSLTSEDSAVYYCARE |
| | | linker positioned in | LGGYWGQGTTLTVSSGGGGSGGGGS |
| | | between the Vl and the | GGGGSQAVVTQESALTTSPGETVTL |
| | | Vh | TCRSSTGAVTTGNYPNWVQEKPDHL |
| | | | FTGLIAGTNNRAPGVPARFSGSLIG |
| | | | DKAALTITGAQTEDEAIYFCALWFS |
| | | | SHWVFGGGTKLTVL |
| | | | (SEQ ID NO: 51) |
| 3 | 15B6 | disulfide-stabilized scFv | QAVVTQESALTTSPGETVTLTCRSS |
| | scdsFv (Vl- | with Cys-substituted mAb | TGAVTTGNYPNWVQEKPDHLFTGLI |
| | lin-Vh) | 15B6 Vl at the N- | AGTNNRAPGVPARFSGSLIGDKAAL |
| | | terminus, the Cys- | TITGAQTEDEAIYFCALWFSSHWVF |
| | | substituted mAb 15B6 Vh | GCGTKLTVLGGGGSGGGGSGGGGSE |
| | | at the C-terminus, and a | VQLQQSGPVLVKPGASVKISCKASG |
| | | [G₄S]₃ linker positioned in | YSFTGYYMHWVRQSLVKCLEWIGRI |
| | | between the Vl and the | NPYTGVPSYKHNFKDKASLTVDKSS |
| | | Vh | STAYMELHSLTSEDSAVYYCARELG |
| | | | GYWGQGTTLTVSS |
| | | | (SEQ ID NO: 52) |
| 4 | 15B6 | disulfide-stabilized scFv | MEVQLQQSGPVLVKPGASVKISCKA |
| | scdsFv (Vh- | with Cys-substituted mAb | SGYSFTGYYMHWVRQSLVKCLEWIG |
| | lin-Vl) | 15B6 Vh at the N- | RINPYTGVPSYKHNFKDKASLTVDK |
| | | terminus, the Cys- | SSSTAYMELHSLTSEDSAVYYCARE |
| | | substituted mAb 15B6 Vl | LGGYWGQGTTLTVSSGGGGSGGGGS |
| | | at the C-terminus, and a | GGGGSQAVVTQESALTTSPGETVTL |
| | | [G₄S]₃ linker positioned in | TCRSSTGAVTTGNYPNWVQEKPDHL |
| | | between the Vl and the | FTGLIAGTNNRAPGVPARFSGSLIG |
| | | Vh | DKAALTITGAQTEDEAIYFCALWFS |
| | | | SHWVFGCGTKLTVL |
| | | | (SEQ ID NO: 53) |
| 5 | 15B6 | scFv with the framework | QVQLQQSGPELEKPGASVKISCKAS |
| | scdsFv (Vh- | region of the Fv of mAb | GYSFTGYYMHWVKQSHGKCLEWIGR |
| | lin-Vl) SS1 | SS1, wherein the CDR | INPYTGVPSYKHNFKDKATLTVDKS |
| | Fr | regions were replaced | SSTAYMDLLSLTSEDSAVYFCAREL |
| | | with the CDRs of mAb | GGYWGQGTTVTVSSGGGGSGGGGSG |
| | | 15B6, with the Vh at the | GGGSDIELTQSPAIMSASPGEKVTM |
| | | N-terminus, the Vl at the | TCRSSTGAVTTGNYPNWYQQKSGTS |
| | | C-terminus, and a [G₄S]₃ | PKGLIAGTNNRAPGVPGRFSGSGSG |
| | | linker positioned in | NSYSLTISSVEAEDDATYYCALWFS |
| | | between the Vl and the | SHWVFGCGTKLEIK |
| | | Vh | (SEQ ID NO: 54) |
| 6 | 15B6 Vh | mAb 15B6 Vh only | EVQLQQSGPVLVKPGASVKISCKAS |
| | | | GYSFTGYYMHWVRQSLVKRLEWIGR |
| | | | INPYTGVPSYKHNFKDKASLTVDKS |
| | | | SSTAYMELHSLTSEDSAVYYCAREL |
| | | | GGYWGQGTTLTVSS |
| | | | (SEQ ID NO: 55) |

TABLE 3

| CAR | CAR | | |
|---|---|---|---|
| Construct | Construct | CAR | |
| No. | Name | Description | CAR Sequence |
| 1 | 15B6 scFv | 15B6 scFv | MQAVVTQESALTTSPGETVTLTCRS |
| | (Vl-lin-Vh) | (Vl-lin-Vh), a | STGAVTTGNYPNWVQEKPDHLFTGL |
| | | CD8 hinge | IAGTNNRAPGVPARFSGSLIGDKAA |
| | | domain, a | LTITGAQTEDEAIYFCALWFSSHWV |
| | | CD8 TM (TM) | FGGGTKLTVLGGGGSGGGGSGGGGS |
| | | domain, a | EVQLQQSGPVLVKPGASVKISCKAS |
| | | 4-1BB | GYSFTGYYMHWVRQSLVKRLEWIGR |
| | | intracellular | INPYTGVPSYKHNFKDKASLTVDKS |
| | | signaling | SSTAYMELHSLTSEDSAVYYCAREL |
| | | domain, and a | GGYWGQGTTLTVSSTSTTTPAPRP |
| | | CD3 zeta | PTPAPTIASQPLSLRPEACRPAAGG |
| | | intracellular | AVHTRGLDFACDIYIWAPLAGTCGV |
| | | signaling | LLLSLVITKRGRKKLLYIFKQPFMR |
| | | domain | PVQTTQEEDGCSCRFPEEEEGGCEL |
| | | | RVKFSRSADAPAYQQGQNQLYNELN |
| | | | LGRREEYDVLDKRRGRDPEMGGKPR |
| | | | RKNPQEGLYNELQKDKMAEAYSEIG |
| | | | MKGERRRGKGHDGLYQGLSTATKDT |
| | | | YDALHMQALPPR |
| | | | (SEQ ID NO: 70) |
| 2 | 15B6 scFv | 15B6 scFv | MEVQLQQSGPVLVKPGASVKISCKA |
| | (Vh-lin-Vl) | (Vh-lin-Vl), a | SGYSFTGYYMHWVRQSLVKRLEWIG |
| | | CD8 hinge | RINPYTGVPSYKHNFKDKASLTVDK |
| | | domain, a | SSSTAYMELHSLTSEDSAVYYCARE |
| | | CD8 TM domain, | LGGYWGQGTTLTVSSGGGGSGGGGS |
| | | a 4-1BB | GGGGSQAVVTQESALTTSPGETVTL |
| | | intracellular | TCRSSTGAVTTGNYPNWVQEKPDHL |
| | | signaling | FTGLIAGTNNRAPGVPARFSGSLIG |
| | | domain, and | DKAALTITGAQTEDEAIYFCALWFS |
| | | a CD3 zeta | SHWVFGGGTKLTVLTSTTTPAPRP |
| | | intracellular | PTPAPTIASQPLSLRPEACRPAAGG |
| | | signaling | AVHTRGLDFACDIYIWAPLAGTCGV |
| | | domain | LLLSLVITKRGRKKLLYIFKQPFMR |
| | | | PVQTTQEEDGCSCRFPEEEEGGCEL |
| | | | RVKFSRSADAPAYQQGQNQLYNELN |
| | | | LGRREEYDVLDKRRGRDPEMGGKPR |
| | | | RKNPQEGLYNELQKDKMAEAYSEIG |
| | | | MKGERRRGKGHDGLYQGLSTATKDT |
| | | | YDALHMQALPPR |
| | | | (SEQ ID NO: 71) |

TABLE 4

| Construct | | Expressed by |
|---|---|---|
| No. | Construct Name | 293T cells? |
| 1 | 15B6 scFv (Vl-lin-Vh) | Yes |
| 2 | 15B6 scFv (Vh-lin-Vl) | Yes |
| 3 | 15B6 scdsFv (Vl-lin-Vh) | Weakly |
| 4 | 15B6 scdsFv (Vh-lin-Vl) | No |
| 5 | 15B6 scdsFv (Vh-lin-Vl) SS1 Fr | No |
| 6 | 15B6 Vh | No |

### EXAMPLE 8

This example demonstrates that a CAR comprising an Fv with the Vl positioned at the N-terminus and the Vh positioned at the C-terminus was more effective in killing KLM1 target cells as compared to the CAR comprising an Fv with the Vh positioned at the N-terminus and the Vl positioned at the C-terminus.

Retroviral vectors encoding CARs comprising the 15B6 scFv (Vl-lin-Vh) construct of Example 7 (Construct No. 1) or the 15B6 scFv (Vh-lin-VI) construct of Example 7 (Construct No. 2) were prepared. Healthy donor PBL were independently transduced with the retroviral vectors to produce CAR-T cells. CAR T-cells were co-cultured with target KLM1 cells.

CAR-T cells including the 15B6 scFv (Vl-lin-Vh) construct of Example 7 were effective in killing KLM1 target cells. The target cell killing provided by the CAR-T cells of including the 15B6 scFv (Vh-lin-Vl) construct of Example 7 was far inferior to that provided by the CAR-T cells including the 15B6 scFv (Vl-lin-Vh) construct and was not useful for killing target cells.

This example demonstrated that the order of the light and heavy chains is important for cytotoxic activity. Only the CAR-T cells prepared with the Vl positioned at the N-terminus and the Vh positioned at the C-terminus provided useful cytotoxicity toward mesothelin expressing cells.

### EXAMPLE 9

This example demonstrates the ability of various humanized mAb 15B6-based single chain constructs to be expressed by T cells.

The Fv regions of the CARs of Examples 4-8 were murine. To be a useful therapeutic agent, the murine Fv region must be humanized. Various humanization strategies were explored. Using various prediction methods, the variable regions of mAb 15B6 were mutated to make the amino acid sequence closer to human. One strategy was to graft the mAb 15B6 CDRs to a humanized framework used to make immunotoxin LMB-100. Another strategy was to mutate amino acid residues in the Fv to match the human germline sequence.

CAR-T vectors encoding CARs comprising any one of 14 different humanized mAb 15B6-based Fv constructs were prepared. The amino acid sequences of the 14 humanized mAb 15B6-based Fv constructs used in the CARs are shown in Table 5. In Table 5, the CDRs are underlined, and the framework regions are in italics.

Each CAR further comprised a CD8 hinge domain, a CD8 TM domain, a 4-1BB intracellular signaling domain, and a CD3 zeta intracellular signaling domain. The full-length amino acid sequences of the CARs of Construct Nos. 9-12 are shown in Table 6. In Table 6, the Fv of the CAR is in bold font, and the TM and T-cell activation domain are italicized.

293T cells were independently transduced with the CAR-T vectors. Expression of the Fv by the 293T cells was tested by a mesothelin binding assay, as described in Example 7.

The results are shown in Table 7. As shown in Table 7, grafting the mAb 15B6 CDRs to a humanized framework used to make immunotoxin LMB-100 (Construct Nos. 7 and 8) were not expressed by 293T cells. Another strategy was to mutate amino acid residues in the Fv to match the human germline sequence (Construct Nos. 9-20). Many of these designs were not expressed by 293T cells (Construct Nos. 13-20), but a few designs were expressed by 293T cells (Construct Nos 9-12). Only the CARs comprising the Fv of Construct Nos. 9, 10, 11, or 12 were expressed by 293T cells.

TABLE 5

| Fv | Fv | | |
|---|---|---|---|
| Construct | Construct | | |
| No. | Name | Fv Description | Fv Sequence |
| 7 | hu15B6 scFv | scFv with the | DIQMTQSPSSLSASVGDRVTITCRSSTGAVTTGNYPNWYQQKSGKAPKG |
| | (Vl-lin- | framework | LIAGTNNRAPGVPSRFSGSGSGTDFTLTISSLQPEDFATYYCALWFSSHW |
| | Vh) | region of | VFGQGTKLEIKGGGGSGGGGSGGGGSQVQLVQSGAEVKKPGASVKVS |
| | (LMB100 Fr) | the Fv of | CKASGYSFTGYYMHWVRQAPGQGLEWIGRINPYTGVPSYKHNFKDKAT |
| | | LMB-100, | MTVDTSTSTVYMELSSLRSEDTAVYYCARELGGYWGQGTLVTVSS |
| | | wherein the | (SEQ ID NO: 56) |
| | | CDR regions were | |
| | | replaced | |
| | | with the | |
| | | CDRs of mAb 15B6, | |
| | | with the Vl at | |
| | | the N-terminus, | |
| | | the Vh at the C- | |
| | | terminus, and a | |
| | | [G₄S]₃ linker | |
| | | positioned in | |
| | | between the | |
| | | Vl and | |
| | | the Vh | |
| 8 | hu15B6 scFv | scFv with the | QVQLVQSGAEVKKPGASVKVSCKASGYSFTGYYMHWVRQAPGQGLEWI |
| | (Vh-lin-Vl) | framework | GRINPYTGVPSYKHNFKDKATMTVDTSTSTVYMELSSLRSEDTAVYYCAR |
| | (LMB100 Fr) | region of | ELGGYWGQGTLVTVSSGGGGSGGGGSGGGGSDIQMTQSPSSLSASVG |
| | | the Fv of | DRVTITCRSSTGAVTTGNYPNWYQQKSGKAPKGLIAGTNNRAPGVPSRF |
| | | LMB-100, | SGSGSGTDFTLTISSLQPEDFATYYCALWFSSHWVFGQGTKLEIK |
| | | wherein the | (SEQ ID NO: 57) |
| | | CDR regions were | |
| | | replaced | |
| | | with the | |
| | | CDRs of mAb 15B6, | |
| | | with the Vh at | |
| | | the N-terminus, | |
| | | the Vl at the C- | |
| | | terminus, and a | |
| | | [G₄S]₃ linker | |
| | | positioned in | |
| | | between the | |
| | | Vl and | |
| | | the Vh | |
| 9 | hu15B6 scFv | scFv with | QAVVTQEPSLTVSPGGTVTLTCASSTGAVTTGNYPNWFQEKPGQAFRGL |
| | (huVL1- | humanized mAb | IAGTNNRAPWVPARFSGSLIGDKAALTLSGVQPEDEAEYFCALWFSSHW |
| | lin-huVH1) | 15B6 Vl | VFGGGTKLTVLGGGGSGGGGSGGGGSEVQLVQSGAEVKKPGASVKVS |
| | | at the | CKASGYSFTGYYMHWVRQAPGQGLEWIGRINPYTGVPSYKHKFQGRVT |
| | | N-terminus, | LTVDKSTSTAYMELSSLRSEDTAVYYCARELGGYWGQGTTVTVSS |
| | | humanized mAb | (SEQ ID NO: 58) |
| | | 15B6 Vh at the | |
| | | C-terminus, | |
| | | and a | |
| | | [G₄S]₃ linker | |
| | | positioned | |
| | | in between | |
| | | the Vl and | |
| | | the Vh | |
| 10 | hu15B6 scFv | scFv with | QAVVTQEPSLTVSPGGTVTLTCASSTGAVTTGNYPNWFQEKPGQAFRGL |
| | (huVL1- | humanized mAb | IAGTNNRAPWVPARFSGSLIGDKAALTLSGVQPEDEAEYFCALWFSSHW |
| | lin-huVH2) | 15B6 Vl | VFGGGTKLTVLGGGGSGGGGSGGGGSQVQLVQSGAEVKKPGASVKVS |
| | | at the | CKASGYSFTGYYMHWVRQAPGQGLEWMGRINPYTGVPSYKHKFQGRV |
| | | N-terminus, | TMTVDKSTSTAYMELSSLRSEDTAVYYCARELGGYWGQGTTVTVSS |
| | | humanized mAb | (SEQ ID NO: 59) |
| | | 15B6 Vh at | |
| | | the C-terminus, | |
| | | and a | |
| | | [G₄S]₃ linker | |
| | | positioned | |
| | | in between | |
| | | the Vl and | |
| | | the Vh | |
| 11 | hu15B6 scFv | scFv with | QAVVTQEPSLTVSPGGTVTLTCASSTGAVTTGNYPNWFQQKPGQAFRG |
| | (huVL2- | humanized mAb | LIAGTNNKASWTPARFSGSLLGDKAALTLSGVQPEDEAEYYCALWFSSH |
| | lin-huVH1) | 15B6 Vl | WVFGGGTKLTVLGGGGSGGGGSGGGGSEVQLVQSGAEVKKPGASVKV |
| | | at the | SCKASGYSFTGYYMHWVRQAPGQGLEWIGRINPYTGVPSYKHKFQGRV |
| | | N-terminus, | TLTVDKSTSTAYMELSSLRSEDTAVYYCARELGGYWGQGTTVTVSS |
| | | humanized mAb | (SEQ ID NO: 60) |
| | | 15B6 Vh | |
| | | at the | |
| | | C-terminus, | |
| | | and a | |
| | | [G₄S]₃ linker | |
| | | positioned | |
| | | in between | |
| | | the Vl and | |
| | | the Vh | |
| 12 | hu15B6 scFv | scFv with | QAVVTQEPSLTVSPGGTVTLTCASSTGAVTTGNYPNWFQQKPGQAFRG |
| | (huVL2- | humanized | LIAGTNNKASWTPARFSGSLLGDKAALTLSGVQPEDEAEYYCALWFSSH |
| | lin-huVH2) | mAb 15B6 Vl | WVFGGGTKLTVLGGGGSGGGGSGGGGSQVQLVQSGAEVKKPGASVKV |
| | | at the N-terminus, | SCKASGYSFTGYYMHWVRQAPGQGLEWMGRINPYTGVPSYKHKFQGR |
| | | humanized mAb | VTMTVDKSTSTAYMELSSLRSEDTAVYYCARELGGYWGQGTTVTVSS |
| | | 15B6 Vh at | (SEQ ID NO: 61) |
| | | the C-terminus, | |
| | | and a | |
| | | [G₄S]₃ linker | |
| | | positioned | |
| | | in between | |
| | | th14e Vl and | |
| | | the Vh | |
| 13 | >h15B6 | scFv with | QTVVTQEPSLTVSPGGTVTLTCRSSTGAVTTGNYPNWFQQKPGQAPRAL |
| | (Vl-Lin-Vh)- | humanized | IAGTNNRAPWTPARFSGSLIGDKAALTLSGVQPEDEAEYYCALWFSSHW |
| | RD1 | mAb 15B6 Vl | VFGGGTKLTVLGGGGSGGGGGGGGSQVQLVQSGAEVKKPGASVKVS |
| | | at the N-terminus, | CKASGYSFTGYYMHWVRQAPGQGLEWMGRINPYTGVPSYAQKFQGRV |
| | | humanized mAb | TMTVDKSSSTAYMELSRLRSDDTAVYYCARELGGYWGQGTLVTVSS |
| | | 15B6 Vh at the | (SEQ ID NO: 62) |
| | | C-terminus, | |
| | | and a | |
| | | [G₄S]₃ linker | |
| | | positioned in | |
| | | between | |
| | | the Vl and | |
| | | the Vh | |
| 14 | > h15B6 | scFv with | QTVVTQEPSLTVSPGGTVTLTCRSSTGAVTTGNYPNWFQQKPGQAPRAL |
| | (Vl-Lin-Vh)- | humanized mAb | IAGTNNRAPGVPARFSGSLIGDKAALTLSGVQPEDEAEYYCALWFSSHW |
| | RD2 | 15B6 Vl | VFGGGTKLTVLGGGGSGGGGSGGGGSQVQLVQSGAEVKKPGASVKVS |
| | | at the | CKASGYSFTGYYMHWVRQAPGQGLEWMGRINPYTGVPSYAQKFQGRV |
| | | N-terminus, | TMTVDKSSSTAYMELSRLRSDDTAVYYCARELGGYWGQGTLVTVSS |
| | | humanized mAb | (SEQ ID NO: 63) |
| | | 15B6 Vh at the | |
| | | C-terminus, | |
| | | and a | |
| | | [G₄S]₃ linker | |
| | | positioned | |
| | | in between | |
| | | the Vl and | |
| | | the Vh | |
| 15 | > h15B6 | scFv with | QTVITQEPSLTVSPGGTVTLTCRSSTGAVTTGNYPNWFQQKPGQAPRALI |
| | (Vl-Lin-Vh)- | humanized | AGTNNRAPGVPARFSGSLIGDKAALTLSGVQPEDEAEYYCALWFSSHWV |
| | RD3 | mAb 15B6 Vl | FGGGTKLTVLGGGGSGGGGSGGGGSQVQLVQSGAEVKKPGASVKVSC |
| | | at the | KASGYSFTGYYMHWVRQAPGQGLEWMGRINPYTGVPSYAQKFQGRVT |
| | | N-terminus, | MTVDKSSSTAYMELSRLRSDDTAVYYCARELGGYWGQGTLVTVSS |
| | | humanized mAb | (SEQ ID NO: 64) |
| | | 15B6 Vh at the | |
| | | C-terminus, | |
| | | and a | |
| | | [G₄S]₃ linker | |
| | | positioned | |
| | | in between | |
| | | the Vl and | |
| | | the Vh | |
| 16 | > h15B6 | scFv with | QTVVTQEPSLTVSPGGTVTLTCRSSTGAVTTGNYPNWFQQKPGQAPRAII |
| | (Vl-Lin-Vh)- | humanized mAb | AGTNNRAPGVPARFSGSLIGDKAALTISGVQPEDEAEYYCALWFSSHWV |
| | RD6 | 15B6 Vl | FGGGTKLTVLGGGGSGGGGSGGGGSQVQLVQSGAEVKKPGASVKVSC |
| | | at the | KASGYSFTGYYMHWVRQAPGQGLEWMGRINPYTGVPSYAQKFQGRVT |
| | | N-terminus, | MTVDKSSSTAYMELSRLRSDDTAVYYCARELGGYWGQGTLVTVSS |
| | | humanized mAb | (SEQ ID NO: 65) |
| | | 15B6 Vh at the | |
| | | C-terminus, | |
| | | and a | |
| | | [G4S]3 linker | |
| | | positioned | |
| | | in between | |
| | | the Vl and | |
| | | the Vh | |
| P17 | > h15B6 | scFv with | QTVVTQEPSLTVSPGGTVTLTCRSSTGAVTTGNYPNWFQQKPGQAPRAL |
| | (Vl-Lin-Vh)- | humanized mAb | IAGTNNRAPGVPARFSGSLIGDKAALTLSGVQPEDEAEYYCALWFSSHW |
| | RD9 | 15B6 Vl | VFGGGTKLTVLGGGGSGGGGSGGGGSQVQLVQSGAEVKKPGASVKVS |
| | | at the | CKASGYSFTGYYMHWVRQAPGQGLEWMGRINPYTGVPSYAQKFQGRV |
| | | N-terminus, | TLTVDKSSSTAYMELSRLRSDDTAVYYCARELGGYWGQGTLVTVSS |
| | | humanized mAb | (SEQ ID NO: 66) |
| | | 15B6 Vh at the | |
| | | C-terminus, | |
| | | and a | |
| | | [G₄S]₃ linker | |
| | | positioned | |
| | | in between | |
| | | the Vl and | |
| | | the Vh | |
| 18 | > h15B6 | scFv with | QTVVTQEPSLTVSPGGTVTLTCRSSTGAVTTGNYPNWFQQKPGQAPRAL |
| | (Vl-Lin-Vh)- | humanized mAb | IAGTNNRAPGVPARFSGSLIGDKAALTLSGVQPEDEAEYYCALWFSSHW |
| | RD11 | 15B6 Vl | VFGGGTKLTVLGGGGSGGGGSGGGGSQVQLVQSGAEVKKPGASVKVS |
| | | at the | CKASGYSFTGYYMHWVRQVPGQGLEWMGRINPYTGVPSYAQKFQGRV |
| | | N-terminus, | TMTVDKSSSTAYMEISRLRSDDTAVYYCARELGGYWGQGTLVTVSS |
| | | humanized mAb | (SEQ ID NO: 67) |
| | | 15B6 Vh at the | |
| | | C-terminus, | |
| | | and a | |
| | | [G₄S]₃ linker | |
| | | positioned | |
| | | in between | |
| | | the Vl and | |
| | | the Vh | |
| 19 | > h15B6 | scFv with | QTVVTQEPSLTVSPGGTVTLTCRSSTGAVTTGNYPNWFQQKPGQAPRAII |
| | (Vl-Lin-Vh)- | humanized | AGTNNRAPGVPARFSGSLIGDKAALTISGVQPEDEAEYYCALWFSSHWV |
| | RD12 | mAb 15B6 Vl | FGGGTKLTVLGGGGSGGGGSGGGGSQVQLVQSGAEVKKPGASVKVSC |
| | | at the | KASGYSFTGYYMHWVRQVPGQGLEWMGRINPYTGVPSYAQKFQGRVT |
| | | N-terminus, | MTVDKSSSTAYMEISRLRSDDTAVYYCARELGGYWGQGTLVTVSS |
| | | humanized mAb | (SEQ ID NO: 68) |
| | | 15B6 Vh at the | |
| | | C-terminus, | |
| | | and a | |
| | | [G₄S]₃ linker | |
| | | positioned | |
| | | in between | |
| | | the Vl and | |
| | | the Vh | |
| 20 | > h15B6 | scFv with | QTVITQEPSLTVSPGGTVTLTCRSSTGAVTTGNYPNWFQQKPGQAPRAII |
| | (Vl-Lin-Vh)- | humanized | AGTNNRAPGVPARFSGSLIGDKAALTISGVQPEDEAEYYCALWFSSHWV |
| | RD13 | mAb 15B6 Vl | FGGGTKLTVLGGGGSGGGGSGGGGSQVQLVQSGAEVKKPGASVKISCK |
| | | at the | ASGYSFTGYYMHWVRQVPGQGLEWMGRINPYTGVPSYAQKFQGRVTLT |
| | | N-terminus, | VDKSSSTAYMEISRLRSDDTAVYYCARELGGYWGQGTLVTVSS |
| | | humanized mAb | (SEQ ID NO: 69) |
| | | 15B6 Vh at the | |
| | | C-terminus, | |
| | | and a | |
| | | [G₄S]₃ linker | |
| | | positioned | |
| | | in between | |
| | | the Vl and | |
| | | the Vh | |

TABLE 6

| CAR | CAR | | |
|---|---|---|---|
| Construct | Construct | | |
| No. | Name | CAR Description | CAR Sequence |
| 9 | hu15B6 | hu15B6 scFv (huVL1-lin- | MQAVVTQEPSLTVSPGGTVTLTCASSTGAV |
| | scFv | huVH1), a CD8 hinge | TTGNYPNWFQEKPGQAFRGLIAGTNNRAPW |
| | (huVL1-lin- | domain, a CD8 TM | VPARFSGSLIGDKAALTLSGVQPEDEAEYF |
| | huVH1) | domain, a 4-1BB | CALWFSSHWVFGGGTKLTVLGGGGSGGGGS |
| | | intracellular signaling | GGGGSEVQLVQSGAEVKKPGASVKVSCKAS |
| | | domain, and a CD3 zeta | GYSFTGYYMHWVRQAPGQGLEWIGRINPYT |
| | | intracellular signaling | GVPSYKHKFQGRVTLTVDKSTSTAYMELSS |
| | | domain | LRSEDTAVYYCARELGGYWGQGTTVTVSST |
| | | | STTTPAPRPPTPAPTIASQPLSLRPEACRP |
| | | | AAGGAVHTRGLDFACDIYIWAPLAGTCGVL |
| | | | LLSLVITKRGRKKLLYIFKQPFMRPVQTTQ |
| | | | EEDGCSCRFPEEEEGGCELRVKFSRSADAP |
| | | | AYQQGQNQLYNELNLGRREEYDVLDKRRGR |
| | | | DPEMGGKPRRKNPQEGLYNELQKDKMAEAY |
| | | | SEIGMKGERRRGKGHDGLYQGLSTATKDTY |
| | | | DALHMQALPPR |
| | | | (SEQ ID NO: 72) |
| 10 | hu15B6 | hu15B6 scFv (huVL1-lin- | MQAVVTQEPSLTVSPGGTVTLTCASSTGAV |
| | scFv | huVH2), a CD8 hinge | TTGNYPNWFQEKPGQAFRGLIAGTNNRAPW |
| | (huVL1-lin- | domain, a CD8 TM | VPARFSGSLIGDKAALTLSGVQPEDEAEYF |
| | huVH2) | domain, a 4-1BB | CALWFSSHWVFGGGTKLTVLGGGGSGGGGS |
| | | intracellular signaling | GGGGSQVQLVQSGAEVKKPGASVKVSCKAS |
| | | domain, and a CD3 zeta | GYSFTGYYMHWVRQAPGQGLEWMGRINPYT |
| | | intracellular signaling | GVPSYKHKFQGRVTMTVDKSTSTAYMELSS |
| | | domain | LRSEDTAVYYCARELGGYWGQGTTVTVSST |
| | | | STTTPAPRPPTPAPTIASQPLSLRPEACRP |
| | | | AAGGAVHTRGLDFACDIYIWAPLAGTCGVL |
| | | | LLSLVITKRGRKKLLYIFKQPFMRPVQTTQ |
| | | | EEDGCSCRFPEEEEGGCELRVKFSRSADAP |
| | | | AYQQGQNQLYNELNLGRREEYDVLDKRRGR |
| | | | DPEMGGKPRRKNPQEGLYNELQKDKMAEAY |
| | | | SEIGMKGERRRGKGHDGLYQGLSTATKDTY |
| | | | DALHMQALPPR |
| | | | (SEQ ID NO: 73) |
| 11 | hu15B6 | hu15B6 scFv (huVL2-lin- | MQAVVTQEPSLTVSPGGTVTLTCASSTGAV |
| | scFv | huVH1), a CD8 hinge | TTGNYPNWFQQKPGQAFRGLIAGTNNKASW |
| | (huVL2-lin- | domain, a CD8 TM | TPARFSGSLLGDKAALTLSGVQPEDEAEYY |
| | huVH1) | domain, a 4-1BB | CALWFSSHWVFGGGTKLTVLGGGGSGGGGS |
| | | intracellular signaling | GGGGSEVQLVQSGAEVKKPGASVKVSCKAS |
| | | domain, and a CD3 zeta | GYSFTGYYMHWVRQAPGQGLEWIGRINPYT |
| | | intracellular signaling | GVPSYKHKFQGRVTLTVDKSTSTAYMELSS |
| | | domain | LRSEDTAVYYCARELGGYWGQGTTVTVSST |
| | | | STTTPAPRPPTPAPTIASQPLSLRPEACRP |
| | | | AAGGAVHTRGLDFACDIYIWAPLAGTCGVL |
| | | | LLSLVITKRGRKKLLYIFKQPFMRPVQTTQ |
| | | | EEDGCSCRFPEEEEGGCELRVKFSRSADAP |
| | | | AYQQGQNQLYNELNLGRREEYDVLDKRRGR |
| | | | DPEMGGKPRRKNPQEGLYNELQKDKMAEAY |
| | | | SEIGMKGERRRGKGHDGLYQGLSTATKDTY |
| | | | DALHMQALPPR |
| | | | (SEQ ID NO: 74) |
| 12 | hu15B6 | hu15B6 scFv (huVL2-lin- | MQAVVTQEPSLTVSPGGTVTLTCASSTGAV |
| | scFv | huVH2), a CD8 hinge | TTGNYPNWFQQKPGQAFRGLIAGTNNKASW |
| | (huVL2-lin- | domain, a CD8 TM | TPARFSGSLLGDKAALTLSGVQPEDEAEYY |
| | huVH2) | domain, a 4-1BB | CALWFSSHWVFGGGTKLTVLGGGGSGGGGS |
| | | intracellular signaling | GGGGSQVQLVQSGAEVKKPGASVKVSCKAS |
| | | domain, and a CD3 zeta | GYSFTGYYMHWVRQAPGQGLEWMGRINPYT |
| | | intracellular signaling | GVPSYKHKFQGRVTMTVDKSTSTAYMELSS |
| | | domain | LRSEDTAVYYCARELGGYWGQGTTVTVSST |
| | | | STTTPAPRPPTPAPTIASQPLSLRPEACRP |
| | | | AAGGAVHTRGLDFACDIYIWAPLAGTCGVL |
| | | | LLSLVITKRGRKKLLYIFKQPFMRPVQTTQ |
| | | | EEDGCSCRFPEEEEGGCELRVKFSRSADAP |
| | | | AYQQGQNQLYNELNLGRREEYDVLDKRRGR |
| | | | DPEMGGKPRRKNPQEGLYNELQKDKMAEAY |
| | | | SEIGMKGERRRGKGHDGLYQGLSTATKDTY |
| | | | DALHMQALPPR |
| | | | (SEQ ID NO: 75) |

TABLE 7

| Construct | | Expressed by |
|---|---|---|
| No. | Fv Construct Name | 293T cells? |
| 7 | hu15B6 scFv (Vl-lin-Vh) (LMB100 Fr) | No |
| 8 | hu15B6 scFv (Vh-lin-Vl) (LMB100 Fr) | No |
| 9 | hu15B6 scFv (huVL1-lin-huVH1) | Yes |
| 10 | hu15B6 scFv (huVL1-lin-huVH2) | Yes |
| 11 | hu15B6 scFv (huVL2-lin-huVH1) | Yes |
| 12 | hu15B6 scFv (huVL2-lin-huVH2) | Yes |
| 13 | > h15B6(Vl-Lin-Vh)-RD1 | No |
| 14 | > h15B6(Vl-Lin-Vh)-RD2 | No |
| 15 | > h15B6(Vl-Lin-Vh)-RD3 | No |
| 16 | > h15B6(Vl-Lin-Vh)-RD6 | No |
| 17 | > h15B6(Vl-Lin-Vh)-RD9 | No |
| 18 | > h15B6(Vl-Lin-Vh)-RD11 | No |
| 19 | > h15B6(Vl-Lin-Vh)-RD12 | No |
| 20 | > h15B6(Vl-Lin-Vh)-RD13 | No |

### EXAMPLE 10

This example demonstrates that cells expressing a CAR comprising the Fv of Construct No 9, 10, 11, or 12 are effective in killing target cells.

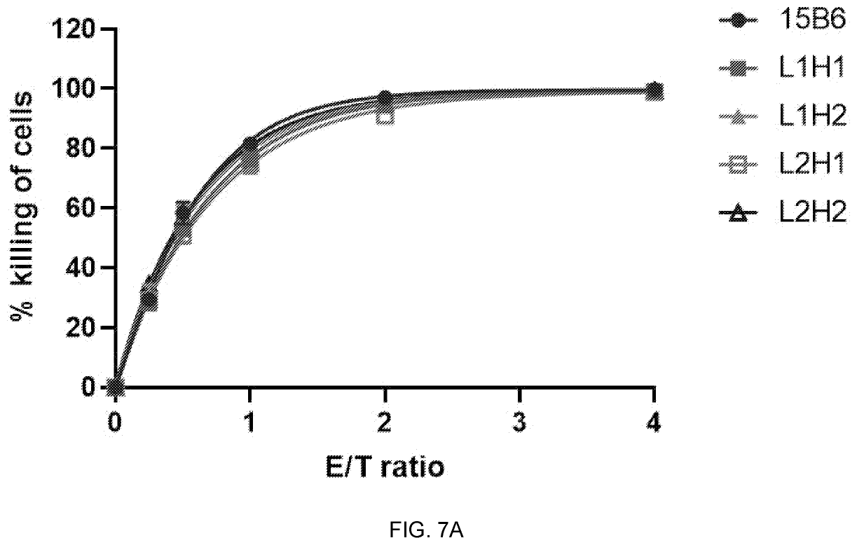

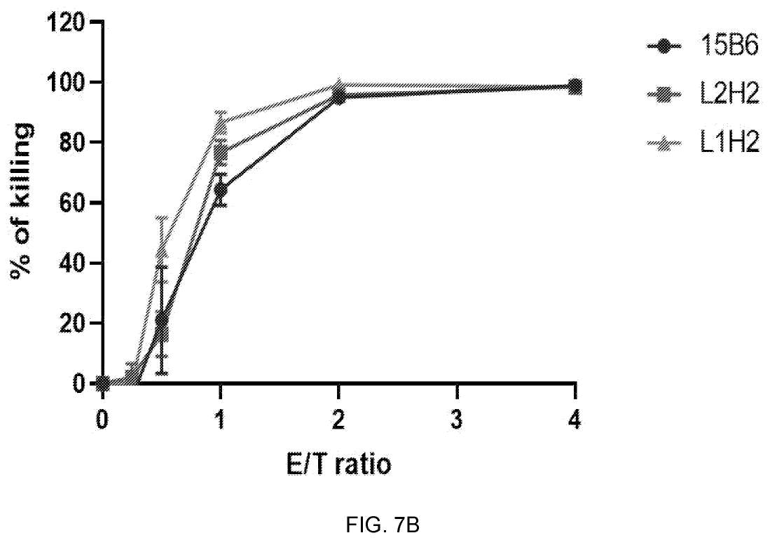

CAR-T vectors encoding CARs comprising the Fv of Construct No 9, 10, 11, or 12 of Table 5 were prepared. Each CAR further comprised a CD8 hinge domain, a CD8 TM domain, a 4-1BB intracellular signaling domain, and a CD3 zeta intracellular signaling domain. Healthy donor PBMC were independently transduced with the vectors to produce CAR-T cells. CAR T-cells were co-cultured with target OVCAR8 or RH29 cells. The results are shown in FIGS. 7A-7B.

Cells expressing a CAR comprising the Fv of Construct No 9, 10, 11, or 12 were effective in killing target cells.

### EXAMPLE 11

This example demonstrates the preparation of nucleic acid constructs encoding a mAb 15B6-based CAR and truncated human epidermal growth factor receptor (huEGFRt).

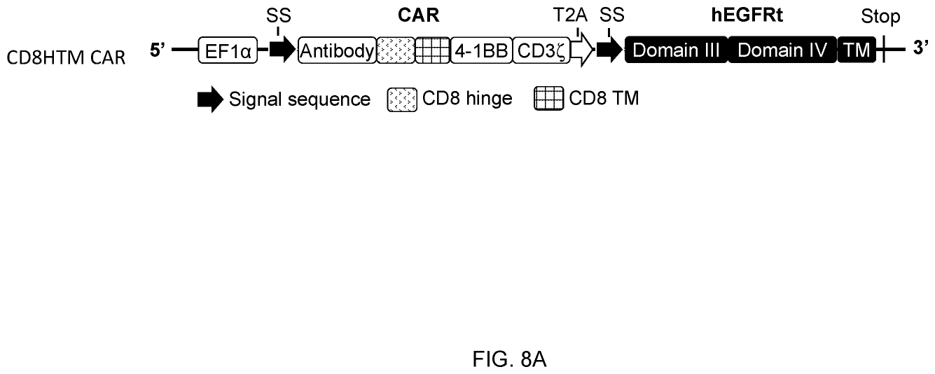

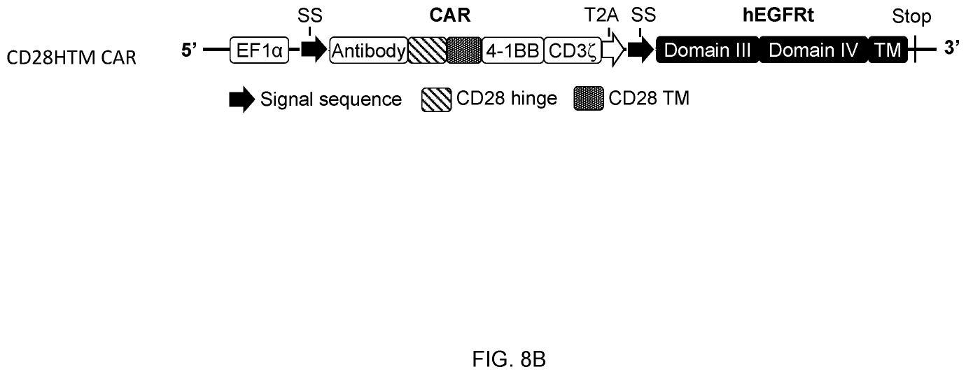

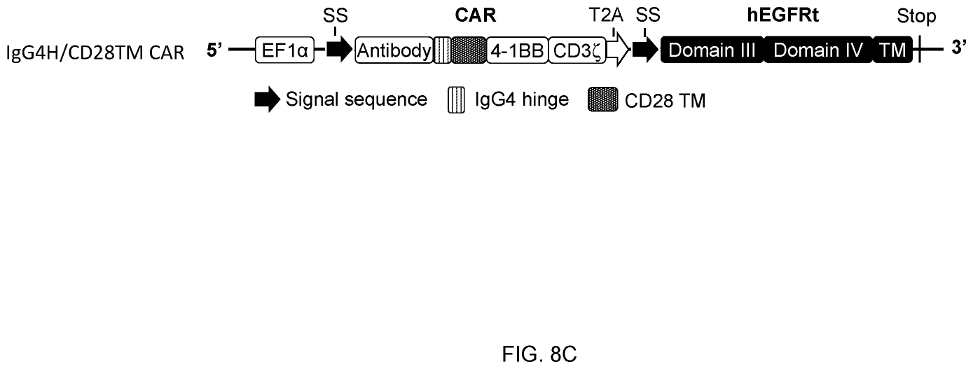

Nucleic acid constructs will be made which encode any of the mAb 15B6-based CARs described herein and a truncated human epidermal growth factor receptor (huEGFRt), as described in WO 2019/094482. FIG. 8A is a schematic showing the general structure of such a nucleic acid construct. Examples of full-length amino acid sequences encoded by such constructs are shown in Table 8.

TABLE 8
15B6 (VL-VH)-CD8HTM CAR
MLLLVTSLLLCELPHPAFLLIPHMQAVVTQESALT
TSPGETVTLTCRSSTGAVTTGNYPNWVQEKPDHLF
TGLIAGTNNRAPGVPARFSGSLIGDKAALTITGAQ
TEDEAIYFCALWFSSHWVFGGGTKLTVLGGGGSGG
GGSGGGGSEVQLQQSGPVLVKPGASVKISCKASGY
SFTGYYMHWVRQSLVKRLEWIGRINPYTGVPSYKH
NFKDKASLTVDKSSSTAYMELHSLTSEDSAVYYCA
RELGGYWGQGTTLTVSSTSTTTPAPRPPTPAPTI
ASQPLSLRPEACRPAAGGAVHTRGLDFACDIYIWA
PLAGTCGVLLLSLVITKRGRKKLLYIFKQPFMRPV
QTTQEEDGCSCRFPEEEEGGCELRVKFSRSADAPA
YQQGQNQLYNELNLGRREEYDVLDKRRGRDPEMGG
KPRRKNPQEGLYNELQKDKMAEAYSEIGMKGERRR
GKGHDGLYQGLSTATKDTYDALHMQALPPREGRGS
LLTCGDVEENPGPMLLLVTSLLLCELPHPAFLLIP
RKVCNGIGIGEFKDSLSINATNIKHFKNCTSISGD
LHILPVAFRGDSFTHTPPLDPQELDILKTVKEITG
FLLIQAWPENRTDLHAFENLEIIRGRTKQHGQFSL
AVVSLNITSLGLRSLKEISDGDVIISGNKNLCYAN
TINWKKLFGTSGQKTKIISNRGENSCKATGQVCHA
LCSPEGCWGPEPRDCVSCRNVSRGRECVDKCNLLE
GEPREFVENSECIQCHPECLPQAMNITCTGRGPDN
CIQCAHYIDGPHCVKTCPAGVMGENNTLVWKYADA
GHVCHLCHPNCTYGCTGPGLEGCPTNGPKIPSIAT
GMVGALLLLLVVALGIGLFM (SEQ ID NO: 76)
15B6 (VH-VL)-CD8HTM CAR
MLLLVTSLLLCELPHPAFLLIPHMEVQLQQSGPVL
VKPGASVKISCKASGYSFTGYYMHWVRQSLVKRLE
WIGRINPYTGVPSYKHNFKDKASLTVDKSSSTAYM
ELHSLTSEDSAVYYCARELGGYWGQGTTLTVSSGG
GGSGGGGSGGGGSQAVVTQESALTTSPGETVTLTC
RSSTGAVTTGNYPNWVQEKPDHLFTGLIAGTNNRA
PGVPARFSGSLIGDKAALTITGAQTEDEAIYFCAL
WFSSHWVFGGGTKLTVLTSTTTPAPRPPTPAPTIA
SQPLSLRPEACRPAAGGAVHTRGLDFACDIYIWAP
LAGTCGVLLLSLVITKRGRKKLLYIFKQPFMRPVQ
TTQEEDGCSCRFPEEEEGGCELRVKFSRSADAPAY
QQGQNQLYNELNLGRREEYDVLDKRRGRDPEMGGK
PRRKNPQEGLYNELQKDKMAEAYSEIGMKGERRRG
KGHDGLYQGLSTATKDTYDALHMQALPPREGRGSL
LTCGDVEENPGPMLLLVTSLLLCELPHPAFLLIPR
KVCNGIGIGEFKDSLSINATNIKHFKNCTSISGDL
HILPVAFRGDSFTHTPPLDPQELDILKTVKEITGF
LLIQAWPENRTDLHAFENLEIIRGRTKQHGQFSLA
VVSLNITSLGLRSLKEISDGDVIISGNKNLCYANT
INWKKLFGTSGQKTKIISNRGENSCKATGQVCHAL
CSPEGCWGPEPRDCVSCRNVSRGRECVDKCNLLEG
EPREFVENSECIQCHPECLPQAMNITCTGRGPDNC
IQCAHYIDGPHCVKTCPAGVMGENNTLVWKYADAG
HVCHLCHPNCTYGCTGPGLEGCPTNGPKIPSIATG
MVGALLLLLVVALGIGLFM (SEQ ID NO: 77)

The individual components of the full-length amino acid sequences of Table 8 are shown in Table 9.

TABLE 9

| Component of CAR | |
|---|---|
| construct of Table 8 | Amino acid sequence |
| GMCSFR signal sequence | MLLLVTSLLLCELPHPAFLLIP (SEQ ID NO: 78) |
| restriction | HM |
| enzyme site | |
| 15B6 (VL-VH) scFv | QAVVTQESALTTSPGETVTLTCRSSTGAVTTGNYPN |
| | WVQEKPDHLFTGLIAGTNNRAPGVPARFSGSLIGDK |
| | AALTITGAQTEDEAIYFCALWFSSHWVFGGGTKLT |
| | VLGGGGSGGGGSGGGGSEVQLQQSGPVLVKPGAS |
| | VKISCKASGYSFTGYYMHWVRQSLVKRLEWIGRIN |
| | PYTGVPSYKHNFKDKASLTVDKSSSTAYMELHSLT |
| | SEDSAVYYCARELGGYWGQGTTLTVSS (SEQ ID |
| | NO: 50) |
| 15B6 (VH-VL) scFv | EVQLQQSGPVLVKPGASVKISCKASGYSFTGYYMH |
| | WVRQSLVKRLEWIGRINPYTGVPSYKHNFKDKASL |
| | TVDKSSSTAYMELHSLTSEDSAVYYCARELGGYWG |
| | QGTTLTVSSGGGGSGGGGSGGGGSQAVVTQESALT |
| | TSPGETVTLTCRSSTGAVTTGNYPNWVQEKPDHLF |
| | TGLIAGTNNRAPGVPARFSGSLIGDKAALTITGAQT |
| | EDEAIYFCALWFSSHWVFGGGTKLTVL (SEQ ID |
| | NO: 79) |
| restriction | TS |
| enzyme site | |
| CD8α hinge | TTTPAPRPPTPAPTIASQPLSLRPEACRPAAGGAVHT |
| | RGLDFACD (SEQ ID NO: 80) |
| CD8α TM domain | IYIWAPLAGTCGVLLLSLVIT (SEQ ID NO: 81) |
| 4-1BB | KRGRKKLLYIFKQPFMRPVQTTQEEDGCSCRFPEEE |
| co-stimulatory | EGGCEL (SEQ ID NO: 82) |
| domain | |
| CDζ signaling | RVKFSRSADAPAYQQGQNQLYNELNLGRREEYDV |
| domain | LDKRRGRDPEMGGKPRRKNPQEGLYNELQKDKMA |
| | EAYSEIGMKGERRRGKGHDGLYQGLSTATKDTYD |
| | ALHMQALPPR (SEQ ID NO: 83) |
| T2A linker | EGRGSLLTCGDVEENPGP (SEQ ID NO: 84) |
| GMCSFR signal | MLLLVTSLLLCELPHPAFLLIP (SEQ ID NO: 78) |
| sequence | RKVCNGIGIGEFKDSLSINATNIKHFKNCTSISGDLHI |
| hEGFRt | LPVAFRGDSFTHTPPLDPQELDILKTVKEITGFLLIQA |
| | WPENRTDLHAFENLEIIRGRTKQHGQFSLAVVSLNI |
| | TSLGLRSLKEISDGDVIISGNKNLCYANTINWKKLFG |
| | TSGQKTKIISNRGENSCKATGQVCHALCSPEGCWGP |
| | EPRDCVSCRNVSRGRECVDKCNLLEGEPREFVENSE |
| | CIQCHPECLPQAMNITCTGRGPDNCIQCAHYIDGPH |
| | CVKTCPAGVMGENNTLVWKYADAGHVCHLCHPN |
| | CTYGCTGPGLEGCPTNGPKIPSIATGMVGALLLLLV |
| | VALGIGLFM (SEQ ID NO: 85) |

Nucleic acid constructs encoding CARs will also be made in which the CD8 hinge region shown in FIG. 8A is replaced with a CD28 hinge region and the CD8 TM domain shown in FIG. 8A is replaced with the CD28 TM domain. FIG. 8B is a schematic showing the general structure of an illustrative example of one of these CARs. Examples of full-length amino acid sequences encoded by such constructs are shown in Table 10.

TABLE 10
15B6 (VL-VH)-CD28HTM CAR
MLLLVTSLLLCELPHPAFLLIPHMQAVVTQESALT
TSPGETVTLTCRSSTGAVTTGNYPNWVQEKPDHLF
TGLIAGTNNRAPGVPARFSGSLIGDKAALTITGAQ
TEDEAIYFCALWFSSHWVFGGGTKLTVLGGGGSGG
GGSGGGGSEVQLQQSGPVLVKPGASVKISCKASGY
SFTGYYMHWVRQSLVKRLEWIGRINPYTGVPSYKH
NFKDKASLTVDKSSSTAYMELHSLTSEDSAVYYCA
RELGGYWGQGTTLTVSSTSIEVMYPPPYLDNEKSN
GTIIHVKGKHLCPSPLFPGPSKPFWVLVVVGGVLA
CYSLLVTVAFIIFWVKRGRKKLLYIFKQPFMRPVQ
TTQEEDGCSCRFPEEEEGGCELRVKFSRSADAPAY
QQGQNQLYNELNLGRREEYDVLDKRRGRDPEMGGK
PRRKNPQEGLYNELQKDKMAEAYSEIGMKGERRRG
KGHDGLYQGLSTATKDTYDALHMQALPPREGRGSL
LTCGDVEENPGPMLLLVTSLLLCELPHPAFLLIP
RKVCNGIGIGEFKDSLSINATNIKHFKNCTSISGD
LHILPVAFRGDSFTHTPPLDPQELDILKTVKEITG
FLLIQAWPENRTDLHAFENLEIIRGRTKQHGQFSL
AVVSLNITSLGLRSLKEISDGDVIISGNKNLCYAN
TINWKKLFGTSGQKTKIISNRGENSCKATGQVCHA
LCSPEGCWGPEPRDCVSCRNVSRGRECVDKCNLLE
GEPREFVENSECIQCHPECLPQAMNITCTGRGPDN
CIQCAHYIDGPHCVKTCPAGVMGENNTLVWKYADA
GHVCHLCHPNCTYGCTGPGLEGCPTNGPKIPSIAT
GMVGALLLLLVVALGIGLFM (SEQ ID NO: 86)
15B6 (VH-VL)-CD28HTM CAR
MLLLVTSLLLCELPHPAFLLIPHMEVQLQQSGPVL
VKPGASVKISCKASGYSFTGYYMHWVRQSLVKRLE
WIGRINPYTGVPSYKHNFKDKASLTVDKSSSTAYM
ELHSLTSEDSAVYYCARELGGYWGQGTTLTVSSGG
GGSGGGGSGGGGSQAVVTQESALTTSPGETVTLTC
RSSTGAVTTGNYPNWVQEKPDHLFTGLIAGTNNRA
PGVPARFSGSLIGDKAALTITGAQTEDEAIYFCAL
WFSSHWVFGGGTKLTVLTSIEVMYPPPYLDNEKSN
GTIIHVKGKHLCPSPLFPGPSKPFWVLVVVGGVLA
CYSLLVTVAFIIFWVKRGRKKLLYIFKQPFMRPVQ
TTQEEDGCSCRFPEEEEGGCELRVKFSRSADAPAY
QQGQNQLYNELNLGRREEYDVLDKRRGRDPEMGGK
PRRKNPQEGLYNELQKDKMAEAYSEIGMKGERRRG
KGHDGLYQGLSTATKDTYDALHMQALPPREGRGSL
LTCGDVEENPGPMLLLVTSLLLCELPHPAFLLIPR
KVCNGIGIGEFKDSLSINATNIKHFKNCTSISGDL
HILPVAFRGDSFTHTPPLDPQELDILKTVKEITGF
LLIQAWPENRTDLHAFENLEIIRGRTKQHGQFSLA
VVSLNITSLGLRSLKEISDGDVIISGNKNLCYANT
INWKKLFGTSGQKTKIISNRGENSCKATGQVCHAL
CSPEGCWGPEPRDCVSCRNVSRGRECVDKCNLLEG
EPREFVENSECIQCHPECLPQAMNITCTGRGPDNC
IQCAHYIDGPHCVKTCPAGVMGENNTLVWKYADAG
HVCHLCHPNCTYGCTGPGLEGCPTNGPKIPSIATG
MVGALLLLLVVALGIGLFM
(SEQ ID NO: 87)

The individual components of the full-length amino acid sequences of Table 10 are shown in Table 11.

TABLE 11

| Component of CAR | |
|---|---|
| construct of Table 10 | Amino acid sequence |
| GMCSFR signal | MLLLVTSLLLCELPHPAFLLIP |
| sequence | (SEQ ID NO: 78) |
| restriction enzyme | HM |
| site | |
| 15B6 (VL-VH) scFv | QAVVTQESALTTSPGETVTLTC |
| | RSSTGAVTTGNYPNWVQEKPDH |
| | LFTGLIAGTNNRAPGVPARFSG |
| | SLIGDKAALTITGAQTEDEAIY |
| | FCALWFSSHWVFGGGTKLTVLG |
| | GGGSGGGGSGGGGSEVQLQQSG |
| | PVLVKPGASVKISCKASGYSFT |
| | GYYMHWVRQSLVKRLEWIGRIN |
| | PYTGVPSYKHNFKDKASLTVDK |
| | SSSTAYMELHSLTSEDSAVYYC |
| | ARELGGYWGQGTTLTVSS |
| | (SEQ ID NO: 50) |
| 15B6 (VH-VL) scFv | EVQLQQSGPVLVKPGASVKISC |
| | KASGYSFTGYYMHWVRQSLVKR |
| | LEWIGRINPYTGVPSYKHNFKD |
| | KASLTVDKSSSTAYMELHSLTS |
| | EDSAVYYCARELGGYWGQGTTL |
| | TVSSGGGGSGGGGSGGGGSQAV |
| | VTQESALTTSPGETVTLTCRSS |
| | TGAVTTGNYPNWVQEKPDHLFT |
| | GLIAGTNNRAPGVPARFSGSLI |
| | GDKAALTITGAQTEDEAIYFCA |
| | LWFSSHWVFGGGTKLTVL |
| | (SEQ ID NO: 79) |
| restriction enzyme | TS |
| site | |
| CD28 hinge | IEVMYPPPYLDNEKSNGTIIHV |
| | KGKHLCPSPLFPGPSKP |
| | (SEQ ID NO: 88) |
| CD28 TM domain | FWVLVVVGGVLACYSLLVTVAF |
| | IIFWV |
| | (SEQ ID NO: 89) |
| 4-1BB co-stimulatory | KRGRKKLLYIFKQPFMRPV |
| domain | QTTQEEDGCSCRFPEEEEG |
| | GCEL (SEQ ID NO: 82) |
| CD3ζ signaling domain | RVKFSRSADAPAYQQGQNQLYN |
| | ELNLGRREEYDVLDKRRGRDPE |
| | MGGKPRRKNPQEGLYNELQKDK |
| | MAEAYSEIGMKGERRRGKGHDG |
| | LYQGLSTATKDTYDALHMQALP |
| | PR (SEQ ID NO: 83) |
| T2A linker | EGRGSLLTCGDVEENPGP |
| | (SEQ ID NO: 84) |
| GMCSFR signal | MLLLVTSLLLCELPHPAFLLIP |
| sequence | (SEQ ID NO: 78) |
| hEGFRt | RKVCNGIGIGEFKDSLSINATN |
| | IKHFKNCTSISGDLHILPVAFR |
| | GDSFTHTPPLDPQELDILKTVK |
| | EITGFLLIQAWPENRTDLHAFE |
| | NLEIIRGRTKQHGQFSLAVVSL |
| | NITSLGLRSLKEISDGDVIISG |
| | NKNLCYANTINWKKLFGTSGQK |
| | TKIISNRGENSCKATGQVCHAL |
| | CSPEGCWGPEPRDCVSCRNVSR |
| | GRECVDKCNLLEGEPREFVENS |
| | ECIQCHPECLPQAMNITCTGRG |
| | PDNCIQCAHYIDGPHCVKTCPA |
| | GVMGENNTLVWKYADAGHVCHL |
| | CHPNCTYGCTGPGLEGCPTNGP |
| | KIPSIATGMVGALLLLLVVALG |
| | IGLFM (SEQ ID NO: 85) |

CARs will be made in which the CD8 hinge region shown in FIG. 8A is replaced with an IgG4 hinge region and the CD8 TM domain shown in FIG. 8A is replaced with the CD28 TM domain. FIG. 8C is a schematic showing the general structure of an illustrative example of one of these CARs. Examples of full-length amino acid sequences encoded by such constructs are shown in Table 12.

TABLE 12
15B6 (VL-VH)-IgG4H-CD28 TM CAR
MLLLVTSLLLCELPHPAFLLIPHMQAVVTQESALT
TSPGETVTLTCRSSTGAVTTGNYPNWVQEKPDHLF
TGLIAGTNNRAPGVPARFSGSLIGDKAALTITGAQ
TEDEAIYFCALWFSSHWVFGGGTKLTVLGGGGSGG
GGSGGGGSEVQLQQSGPVLVKPGASVKISCKASGY
SFTGYYMHWVRQSLVKRLEWIGRINPYTGVPSYKH
NFKDKASLTVDKSSSTAYMELHSLTSEDSAVYYCA
RELGGYWGQGTTLTVSSTSESKYGPPCPPCPFWVL
VVVGGVLACYSLLVTVAFIIFWVKRGRKKLLYIFK
QPFMRPVQTTQEEDGCSCRFPEEEEGGCELRVKFS
RSADAPAYQQGQNQLYNELNLGRREEYDVLDKRRG
RDPEMGGKPRRKNPQEGLYNELQKDKMAEAYSEIG
MKGERRRGKGHDGLYQGLSTATKDTYDALHMQALP
PREGRGSLLTCGDVEENPGPMLLLVTSLLLCELPH
PAFLLIPRKVCNGIGIGEFKDSLSINATNIKHFKN
CTSISGDLHILPVAFRGDSFTHTPPLDPQELDILK
TVKEITGFLLIQAWPENRTDLHAFENLEIIRGRTK
QHGQFSLAVVSLNITSLGLRSLKEISDGDVIISGN
KNLCYANTINWKKLFGTSGQKTKIISNRGENSCKA
TGQVCHALCSPEGCWGPEPRDCVSCRNVSRGRECV
DKCNLLEGEPREFVENSECIQCHPECLPQAMNITC
TGRGPDNCIQCAHYIDGPHCVKTCPAGVMGENNTL
VWKY ADAGHVCHLCHPNCTYGCTGPGLEGCPTNG
PKIPSIATGMVGALLLLLVVALGIGLFM
(SEQ ID NO: 90)
15B6 (VH-VL)-IgG4H-CD28 TM CAR
MLLLVTSLLLCELPHPAFLLIPHMEVQLQQSGPVL
VKPGASVKISCKASGYSFTGYYMHWVRQSLVKRLE
WIGRINPYTGVPSYKHNFKDKASLTVDKSSSTAYM
ELHSLTSEDSAVYYCARELGGYWGQGTTLTVSSGG
GGSGGGGSGGGGSQAVVTQESALTTSPGETVTLTC
RSSTGAVTTGNYPNWVQEKPDHLFTGLIAGTNNRA
PGVPARFSGSLIGDKAALTITGAQTEDEAIYFCAL
WFSSHWVFGGGTKLTVLTSESKYGPPCPPCPFWVL
VVVGGVLACYSLLVTVAFIIFWVKRGRKKLLYIFK
QPFMRPVQTTQEEDGCSCRFPEEEEGGCELRVKFS
RSADAPAYQQGQNQLYNELNLGRREEYDVLDKRRG
RDPEMGGKPRRKNPQEGLYNELQKDKMAEAYSEIG
MKGERRRGKGHDGLYQGLSTATKDTYDALHMQALP
PREGRGSLLTCGDVEENPGPMLLLVTSLLLCELPH
PAFLLIPRKVCNGIGIGEFKDSLSINATNIKHFKN
CTSISGDLHILPVAFRGDSFTHTPPLDPQELDILK
TVKEITGFLLIQAWPENRTDLHAFENLEIIRGRTK
QHGQFSLAVVSLNITSLGLRSLKEISDGDVIISGN
KNLCYANTINWKKLFGTSGQKTKIISNRGENSCKA
TGQVCHALCSPEGCWGPEPRDCVSCRNVSRGRECV
DKCNLLEGEPREFVENSECIQCHPECLPQAMNITC
TGRGPDNCIQCAHYIDGPHCVKTCPAGVMGENNTL
VWKYADAGHVCHLCHPNCTYGCTGPGLEGCPTNGP
KIPSIATGMVGALLLLLVVALGIGLFM
(SEQ ID NO: 91)

The individual components of the full-length amino acid sequences of Table 12 are shown in Table 13.

TABLE 13

| Component of CAR | |
|---|---|
| construct of Table 12 | Amino acid sequence |
| GMCSFR signal | MLLLVTSLLLCELPHPAFLLIP |
| sequence | (SEQ ID NO: 78) |
| restriction enzyme | HM |
| site | |
| 15B6 (VL-VH) scFv | QAVVTQESALTTSPGETVTLTC |
| | RSSTGAVTTGNYPNWVQEKPDH |
| | LFTGLIAGTNNRAPGVPARFSG |
| | SLIGDKAALTITGAQTEDEAIY |
| | FCALWFSSHWVFGGGTKLTVLG |
| | GGGSGGGGSGGGGSEVQLQQSG |
| | PVLVKPGASVKISCKASGYSFT |
| | GYYMHWVRQSLVKRLEWIGRIN |
| | PYTGVPSYKHNFKDKASLTVDK |
| | SSSTAYMELHSLTSEDSAVYYC |
| | ARELGGYWGQGTTLTVSS |
| | (SEQ ID |
| | NO: 50) |
| 15B6 (VH-VL) scFv | EVQLQQSGPVLVKPGASVKISC |
| | KASGYSFTGYYMHWVRQSLVKR |
| | LEWIGRINPYTGVPSYKHNFKD |
| | KASLTVDKSSSTAYMELHSLTS |
| | EDSAVYYCARELGGYWGQGTTL |
| | TVSSGGGGSGGGGSGGGGSQAV |
| | VTQESALTTSPGETVTLTCRSS |
| | TGAVTTGNYPNWVQEKPDHLFT |
| | GLIAGTNNRAPGVPARFSGSLI |
| | GDKAALTITGAQTEDEAIYFCA |
| | LWFSSHWVFGGGTKLTVL |
| | (SEQ ID NO: 79) |
| restriction enzyme | TS |
| site | |
| IgG4 hinge | ESKYGPPCPPCP |
| | (SEQ ID NO: 92) |
| CD28 TM domain | FWVLVVVGGVLACYSLLVTVAF |
| | IIFWV |
| | (SEQ ID NO: 89) |
| 4-1BB co-stimulatory | KRGRKKLLYIFKQPFMRPVQTT |
| domain | QEEDGCSCRFPEEE |
| | EGGCEL |
| | (SEQ ID NO: 82) |
| CD3ζ signaling domain | RVKFSRSADAPAYQQGQNQLYN |
| | ELNLGRREEYDVLDKRRGRDPE |
| | MGGKPRRKNPQEGLYNELQKDK |
| | MAEAYSEIGMKGERRRGKGHDG |
| | LYQGLSTATKDTYD |
| | ALHMQALPPR |
| | (SEQ ID NO: 83) |
| T2A linker | EGRGSLLTCGDVEENPGP |
| | (SEQ ID NO: 84) |
| GMCSFR signal sequence | MLLLVTSLLLCELPHPAFLLIP |
| | (SEQ ID NO: 78) |
| hEGFRt | RKVCNGIGIGEFKDSLSINATN |
| | IKHFKNCTSISGDLHILPVAFR |
| | GDSFTHTPPLDPQELDILKTVK |
| | EITGFLLIQAWPENRTDLHAFE |
| | NLEIIRGRTKQHGQFSLAVVSL |
| | NITSLGLRSLKEISDGDVIISG |
| | NKNLCYANTINWKKLFGTSGQK |
| | TKIISNRGENSCKATGQVCHAL |
| | CSPEGCWGPEPRDCVSCRNVSR |
| | GRECVDKCNLLEGEPREFVENS |
| | ECIQCHPECLPQAMNITCTGRG |
| | PDNCIQCAHYIDGPHCVKTCPA |
| | GVMGENNTLVWKYADAGHVCHL |
| | CHPNCTYGCTGPGLEGCPTNGP |
| | KIPSIATGMVGALLLLLVVALG |
| | IGLFM |
| | (SEQ ID NO: 85) |

### EXAMPLE 12

This example demonstrates the preparation of nucleic acid constructs encoding a mAb 15B6-based CAR and a second CAR.

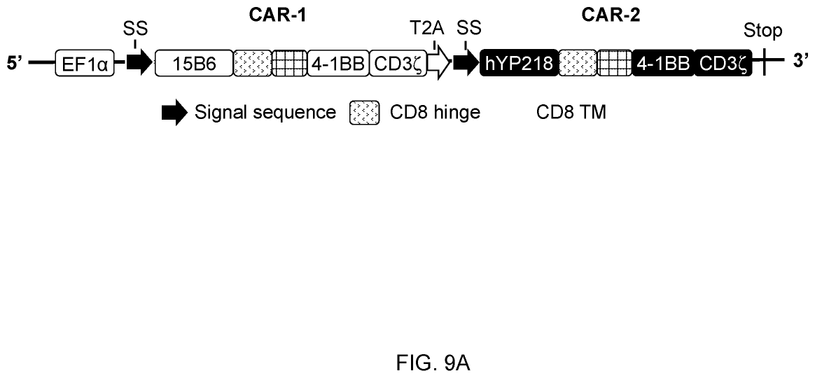

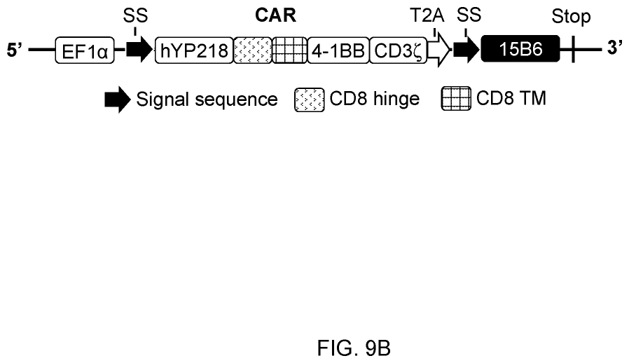

Bicistronic nucleic acid constructs will be made encoding first and second CARs. The first CAR will be a mAb 15B6-based CAR. The second CAR will bind to shed mesothelin. FIG. 9A is a schematic showing the general structure of an illustrative example of one of these bicistronic nucleic acid constructs. In FIG. 9A, CAR-2 includes the Fv of humanized mAb YP218. YP218 has very high affinity but binds to the shed region of mesothelin. It is contemplated that the 15B6-based CAR will slow shedding. Examples of full-length amino acid sequences encoded by such constructs are shown in Table 14.

TABLE 14
Signal peptide-15B6 Vl-lin-Vh-CAR
T2A signal peptide huYP218-CAR

MLLLVTSLLLCELPHPAFLLIPHMQAVVTQESALT
TSPGETVTLTCRSSTGAVTTGNYPNWVQEKPDHLF
TGLIAGTNNRAPGVPARFSGSLIGDKAALTITGAQ
TEDEAIYFCALWFSSHWVFGGGTKLTVLGGGGSGG
GGSGGGGSEVQLQQSGPVLVKPGASVKISCKASGY
SFTGYYMHWVRQSLVKRLEWIGRINPYTGVPSYKH
NFKDKASLTVDKSSSTAYMELHSLTSEDSAVYYCA
RELGGYWGQGTTLTVSSTSTTTPAPRPPTPAPTIA
SQPLSLRPEACRPAAGGAVHTRGLDFACDIYIWAP
LAGTCGVLLLSLVITKRGRKKLLYIFKQPFMRPVQ
TTQEEDGCSCRFPEEEEGGCELRVKFSRSADAPAY
QQGQNQLYNELNLGRREEYDVLDKRRGRDPEMGGK
PRRKNPQEGLYNELQKDKMAEAYSEIGMKGERRRG
KGHDGLYQGLSTATKDTYDALHMQALPPREGRGSL
LTCGDVEENPGPMLLLVTSLLLCELPHPAFLLIPE
VQLVESGGGLVQPGGSLRLSCAASGFDLGFYFYAC
WVRQAPGKGLEWVSCIYTAGSGSTYYASWAKGRFT
ISRDNSKNTLYLQMNSLRAEDTAVYYCARSTANTR
STYYLNLWGQGTLVTVSSGGGGSGGGGSGGGGSDI
QMTQSPSSLSASVGDRVTITCQASQRISSYLSWYQ
QKPGKVPKLLIYGASTLASGVPSRFSGSGSGTDFT
LTISSLQPEDVATYYCQSYAYFDSNNWHAFGGGTK
VEIKTSTTTPAPRPPTPAPTIASQPLSLRPEACRP
AAGGAVHTRGLDFACDIYIWAPLAGTCGVLLLSLV
ITKRGRKKLLYIFKQPFMRPVQTTQEEDGCSCRFP
EEEEGGCELRVKFSRSADAPAYQQGQNQLYNELNL
GRREEYDVLDKRRGRDPEMGGKPRRKNPQEGLYNE
LQKDKMAEAYSEIGMKGERRRGKGHDGLYQGLSTA
TKDTYDALHMQALPPR
(SEQ ID NO: 93)

The individual components of the full-length amino acid sequences of Table 14 are shown in Table 15.

TABLE 15

| Component of CAR | |
|---|---|
| construct of Table 14 | Amino acid sequence |
| GMCSFR signal | MLLLVTSLLLCELPHPAFLLIP |
| sequence | (SEQ ID NO: 78) |
| restriction enzyme | HM |
| site | |
| 15B6 Vl-lin-Vh-CAR | QAVVTQESALTTSPGETVTLTC |
| | RSSTGAVTTGNYPNWVQEKPDH |
| | LFTGLIAGTNNRAPGVPARFSG |
| | SLIGDKAALTITGAQTEDEAIY |
| | FCALWFSSHWVFGGGTKLTVLG |
| | GGGSGGGGSGGGGSEVQLQQSG |
| | PVLVKPGASVKISCKASGYSFT |
| | GYYMHWVRQSLVKRLEWIGRIN |
| | PYTGVPSYKHNFKDKASLTVDK |
| | SSSTAYMELHSLTSEDSAVYYC |
| | ARELGGYWGQGTTLTVSSTSTT |
| | TPAPRPPTPAPTIASQPLSLRP |
| | EACRPAAGGAVHTRGLDFACDI |
| | YIWAPLAGTCGVLLLSLVITKR |
| | GRKKLLYIFKQPFMRPVQTTQE |
| | EDGCSCRFPEEEEGGCELRVKF |
| | SRSADAPAYQQGQNQLYNELNL |
| | GRREEYDVLDKRRGRDPEMGGK |
| | PRRKNPQEGLYNELQKDKMAEA |
| | YSEIGMKGERRRGKGHDGLYQG |
| | LSTATKDTYDALHMQALPPR |
| | (SEQ ID NO: 94) |
| T2A linker | EGRGSLLTCGDVEENPGP |
| | (SEQ ID NO: 84) |
| GMCSFR signal | MLLLVTSLLLCELPHPAFLLIP |
| sequence | (SEQ ID NO: 78) |
| huYP218-CAR | EVQLVESGGGLVQPGGSLRLSC |
| | AASGFDLGFYFYACWVRQAPGK |
| | GLEWVSCIYTAGSGSTYYASWA |
| | KGRFTISRDNSKNTLYLQMNSL |
| | RAEDTAVYYCARSTANTRSTYY |
| | LNLWGQGTLVTVSSGGGGSGGG |
| | GSGGGGSDIQMTQSPSSLSASV |
| | GDRVTITCQASQRISSYLSWYQ |
| | QKPGKVPKLLIYGASTLASGVP |
| | SRFSGSGSGTDFTLTISSLQPE |
| | DVATYYCQSYAYFDSNNWHAFG |
| | GGTKVEIKTSTTTPAPRPPTPA |
| | PTIASQPLSLRPEACRPAAGGA |
| | VHTRGLDFACDIYIWAPLAGTC |
| | GVLLLSLVITKRGRKKLLYIFK |
| | QPFMRPVQTTQEEDGCSCRFPE |
| | EEEGGCELRVKFSRSADAPAYQ |
| | QGQNQLYNELNLGRREEYDVLD |
| | KRRGRDPEMGGKPRRKNPQEGL |
| | YNELQKDKMAEAYSEIGMKGER |
| | RRGKGHDGLYQGLSTATKDTYD |
| | ALHMQALPPR |
| | (SEQ ID NO: 95) |

### EXAMPLE 13

This example demonstrates the preparation of nucleic acid constructs encoding a mAb 15B6-based scFv and a CAR.

Bicistronic vectors will be made encoding a CAR and a 15B6 Fv. The CAR will include the Fv of humanized mAb YP218. FIG. 9B is a schematic showing the general structure of an illustrative example of one of these bicistronic vectors. This construct will use the YP218 Fv to bind to mesothelin and an Fv from 15B6 that will be secreted and reduce or prevent mesothelin shedding. Examples of full-length amino acid sequences encoded by such constructs are shown in Table 16.

TABLE 16
Signal peptide-huYP218-CAR T2A
signal peptide 15B6 Vl-lin-Vh

MLLLVTSLLLCELPHPAFLLIPHMEVQLVESGGGL
VQPGGSLRLSCAASGFDLGFYFYACWVRQAPGKGL
EWVSCIYTAGSGSTYYASWAKGRFTISRDNSKNTL
YLQMNSLRAEDTAVYYCARSTANTRSTYYLNLWGQ
GTLVTVSSGGGGSGGGGSGGGGSDIQMTQSPSSLS
ASVGDRVTITCQASQRISSYLSWYQQKPGKVPKLL
IYGASTLASGVPSRFSGSGSGTDFTLTISSLQPED
VATYYCQSYAYFDSNNWHAFGGGTKVEIKTSTTTP
APRPPTPAPTIASQPLSLRPEACRPAAGGAVHTRG
LDFACDIYIWAPLAGTCGVLLLSLVITKRGRKKLL
YIFKQPFMRPVQTTQEEDGCSCRFPEEEEGGCELR
VKFSRSADAPAYQQGQNQLYNELNLGRREEYDVLD
KRRGRDPEMGGKPRRKNPQEGLYNELOKDKMAEAY
SEIGMKGERRRGKGHDGLYQGLSTATKDTYDALHM
QALPPREGRGSLLTCGDVEENPGPMLLLVTSLLLC
ELPHPAFLLIPQAVVTQESALTTSPGETVTLTCRS
STGAVTTGNYPNWVQEKPDHLFTGLIAGTNNRAPG
VPARFSGSLIGDKAALTITGAQTEDEAIYFCALWF
SSHWVFGGGTKLTVLGGGGSGGGGSGGGGSEVQLQ
QSGPVLVKPGASVKISCKASGYSFTGYYMHWVRQS
LVKRLEWIGRINPYTGVPSYKHNFKDKASLTVDKS
SSTAYMELHSLTSEDSAVYYCARELGGYWGQGTTL
TVSS
(SEQ ID NO: 96)

The individual components of the full-length amino acid sequences of Table 16 are shown in Table 17.

TABLE 17

| Component of CAR | |
|---|---|
| construct of Table 16 | Amino acid sequence |
| GMCSFR signal sequence | MLLLVTSLLLCELPHPAFLLIP |
| | (SEQ ID NO: 78) |
| restriction enzyme site | HM |
| huYP218-CAR | EVQLVESGGGLVQPGGSLRLSC |
| | AASGFDLGFYFYACWVRQAPGK |
| | GLEWVSCIYTAGSGSTYYASWA |
| | KGRFTISRDNSKNTLYLQMNSL |
| | RAEDTAVYYCARSTANTRSTYY |
| | LNLWGQGTLVTVSSGGGGSGGG |
| | GSGGGGSDIQMTQSPSSLSASV |
| | GDRVTITCQASQRISSYLSWYQ |
| | QKPGKVPKLLIYGASTLASGVP |
| | SRFSGSGSGTDFTLTISSLQPE |
| | DVATYYCQSYAYFDSNNWHAFG |
| | GGTKVEIKTSTTTPAPRPPTPA |
| | PTIASQPLSLRPEACRPAAGGA |
| | VHTRGLDFACDIYIWAPLAGTC |
| | GVLLLSLVITKRGRKKLLYIFK |
| | QPFMRPVQTTQEEDGCSCRFPE |
| | EEEGGCELRVKFSRSADAPAYQ |
| | QGQNQLYNELNLGRREEYDVLD |
| | KRRGRDPEMGGKPRRKNPQEGL |
| | YNELQKDKMAEAYSEIGMKGER |
| | RRGKGHDGLYQGLSTATKDTYD |
| | ALHMQALPPR |
| | (SEQ ID NO: 95) |
| T2A linker | EGRGSLLTCGDVEENPGP |
| | (SEQ ID NO: 84) |
| GMCSFR signal sequence | MLLLVTSLLLCELPHPAFLLIP |
| | (SEQ ID NO: 78) |
| 15B6 Vl-lin-Vh | QAVVTQESALTTSPGETVTLTC |
| | RSSTGAVTTGNYPNWVQEKPDH |
| | LFTGLIAGTNNRAPGVPARFSG |
| | SLIGDKAALTITGAQTEDEAIY |
| | FCALWFSSHWVFGGGTKLTVLG |
| | GGGSGGGGSGGGGSEVQLQQSG |
| | PVLVKPGASVKISCKASGYSFT |
| | GYYMHWVRQSLVKRLEWIGRIN |
| | PYTGVPSYKHNFKDKASLTVDK |
| | SSSTAYMELHSLTSEDSAVYYC |
| | ARELGGYWGQGTTLTVSS |
| | (SEQ ID NO: 50) |

### EXAMPLE 14

This example demonstrates that 15B6 CAR-T cells provide complete regression of MD0887 PDC xenografts growing in NSG (NOD scid gamma) mice.

CAR-T cells (effector cells) were produced by transducing healthy human donor PBMC with a retroviral vector encoding a CAR comprising the antigen binding domain of mAb 15B6 (CAR Construct No. 1 of Table 3) (15B6 CAR-T cells). Mock-transfected T cells (no plasmid) was used as a control.

MD0887 PDC xenografts were transplanted into NSG mice on Day 0. This provides a human pancreatic cancer propagated in mice that retains all characteristics of original human tumor. A histology evaluation of the tumors in this mouse model of pancreatic ductal cancer (PDX Model MD0887) showed that the tumors were positive for mesothelin expression.

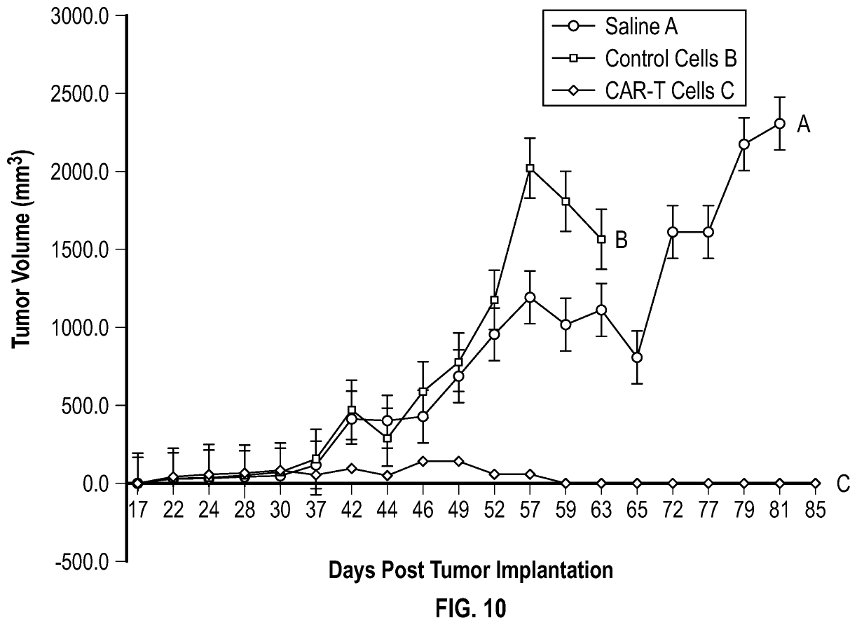

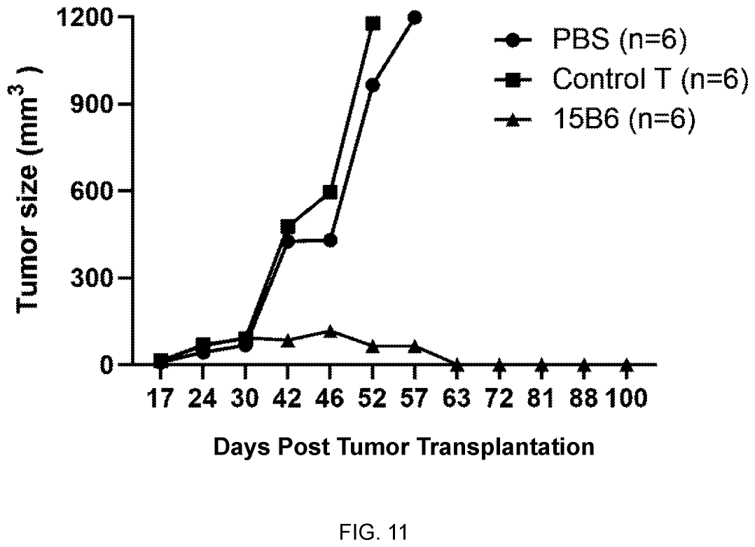

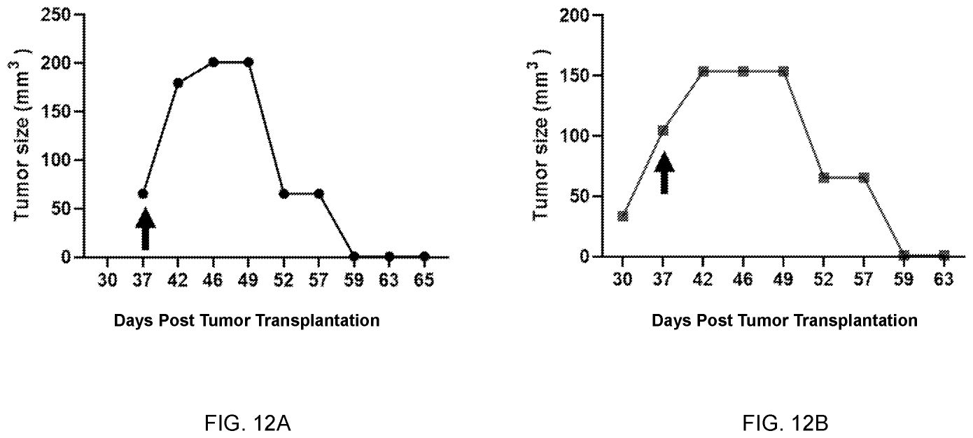

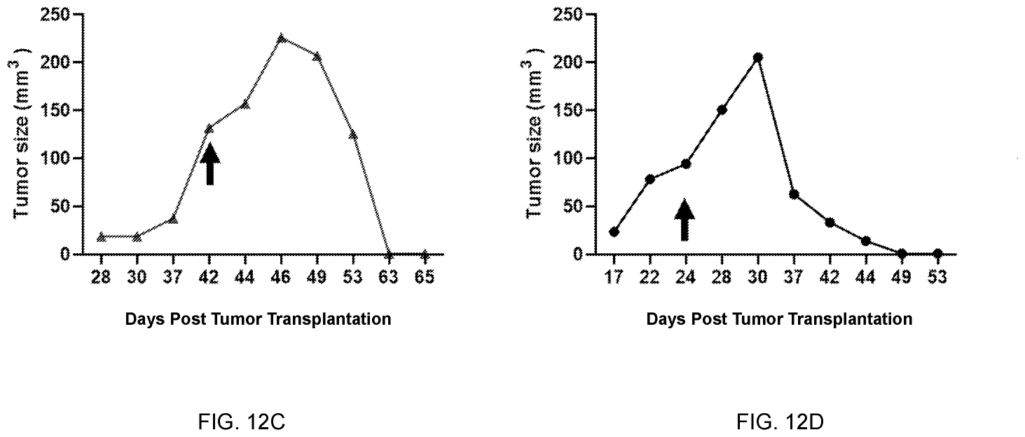

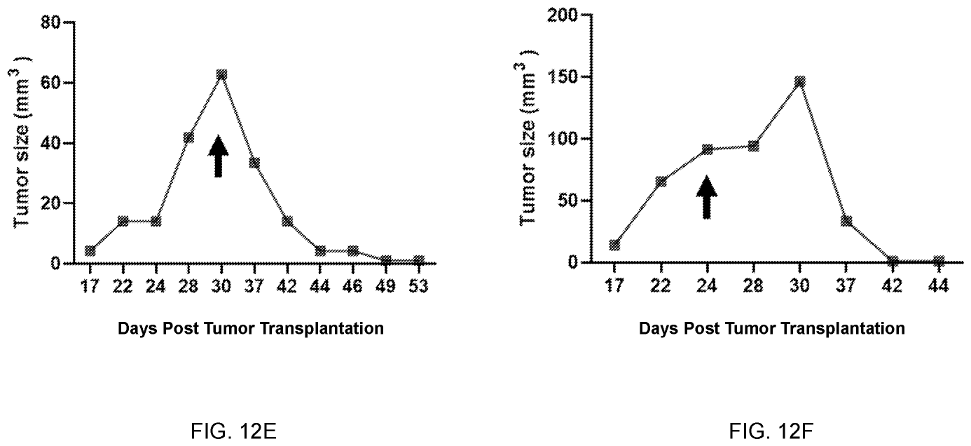

Mice were administered (i) saline, (ii) 10 million 15B6 CAR-T cells, or (iii) 10million control cells (IV) (dorsal or ventral) between Day 24 and Day 42, when tumors were 65 to 125 mm3. The average volume of the tumor was measured for up to 100 days after tumor transplantation. The results (average tumor volume) are shown in FIGS. 10-11. The results for each of one of six individual mice are shown in FIGS. 12A-12F, respectively. The 15B6 CAR T cells provided complete regression of human PDC xenografts growing in NSG mice. To the best of the inventors' knowledge, this is the first time that it has been shown that CAR-T cells are active in a mouse model of human cancer that retains all characteristics of original human tumor.

### EXAMPLE 15

This example demonstrates the structures of various BiTEs according to aspects of the invention.

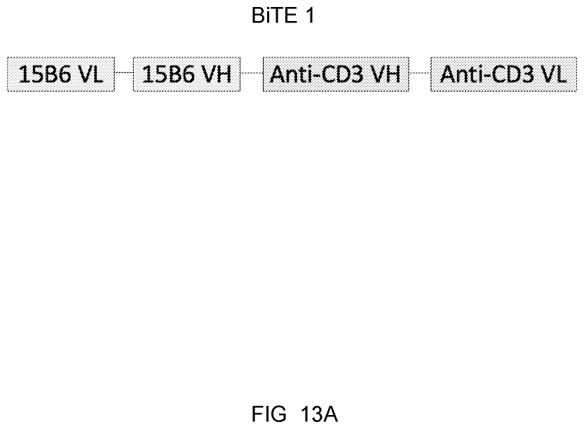

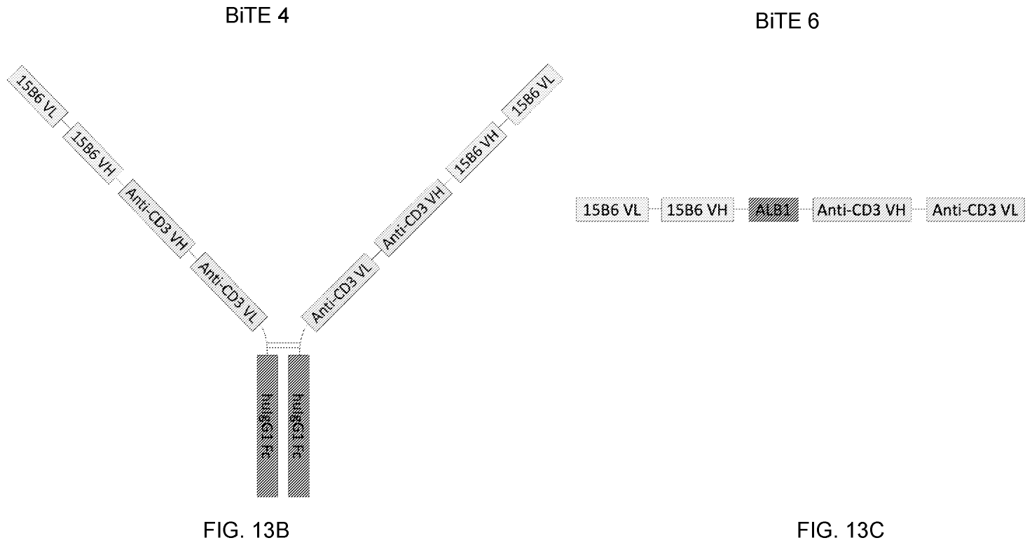

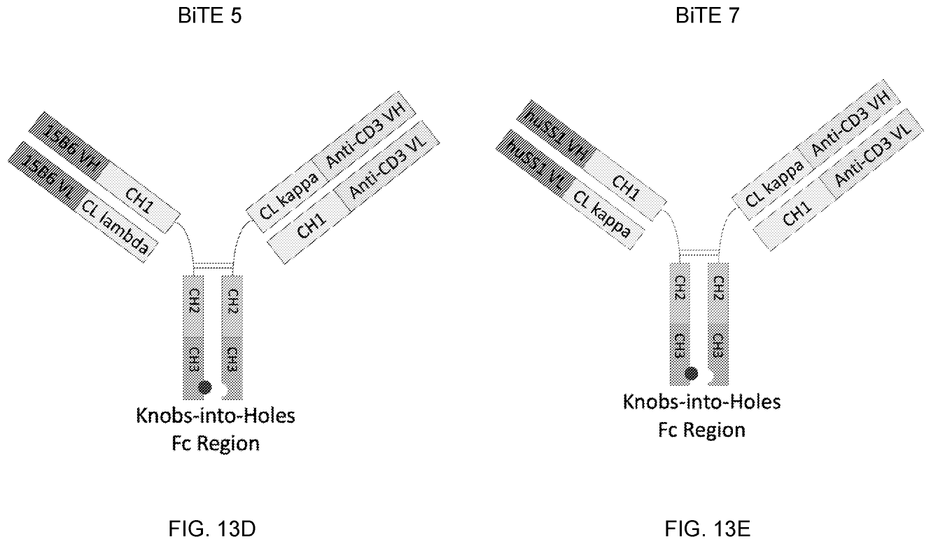

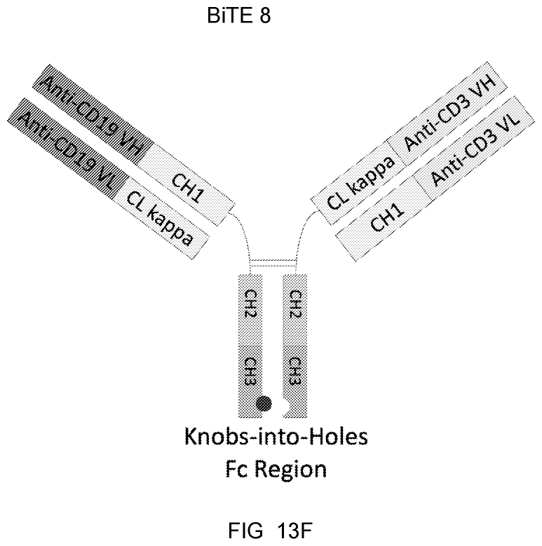

BiTEs in various formats, each comprising 15B6 Fv were prepared. The structures of the 15B6 Fv-based BiTEs are shown in FIGS. 13A-13D. The amino acid sequences of the BiTEs are set forth in Table 18.

TABLE 18

| Name | Amino Acid Sequence |
|---|---|
| BITE 1 | MGWSCIILFLVATATGVHSQ |
| | AVVTQESALTTSPGETVTLT |
| | CRSSTGAVTTGNYPNWVQEK |
| | PDHLFTGLIAGTNNRAPGVP |
| | ARFSGSLIGDKAALTITGAQ |
| | TEDEAIYFCALWFSSHWVFG |
| | GGTKLTVLGGGGSGGGGSGG |
| | GGSEVQLQQSGPVLVKPGAS |
| | VKISCKASGYSFTGYYMHWV |
| | RQSLVKRLEWIGRINPYTGV |
| | PSYKHNFKDKASLTVDKSSS |
| | TAYMELHSLTSEDSAVYYCA |
| | RELGGYWGQGTTLTVSSGGG |
| | GSEVQLVESGGGLVQPGRSL |
| | RLSCAASGFTFDDYTMHWVR |
| | QAPGKGLEWVSGISWNSGSI |
| | GYADSVKGRFTISRDNAKKS |
| | LYLQMNSLRAEDTALYYCAK |
| | DNSGYGHYYYGMDVWGQGTT |
| | VTVASGGGGSGGGGSGGGGS |
| | EIVMTQSPATLSVSPGERAT |
| | LSCRASQSVSSNLAWYQQKP |
| | GQAPRLLIYGASTRATGIPA |
| | RFSGSGSGTEFTLTISSLQS |
| | EDFAVYYCQHYINWPLTFGG |
| | GTKVEIKHHHHHH |
| | (SEQ ID NO: 97) |
| BITE 4 | MGWSCIILFLVATATGVHSQ |
| | AVVTQESALTTSPGETVTLT |
| | CRSSTGAVTTGNYPNWVQEK |
| | PDHLFTGLIAGTNNRAPGVP |
| | ARFSGSLIGDKAALTITGAQ |
| | TEDEAIYFCALWFSSHWVFG |
| | GGTKLTVLGGGGSGGGGSGG |
| | GGSEVQLQQSGPVLVKPGAS |
| | VKISCKASGYSFTGYYMHWV |
| | RQSLVKRLEWIGRINPYTGV |
| | PSYKHNFKDKASLTVDKSSS |
| | TAYMELHSLTSEDSAVYYCA |
| | RELGGYWGQGTTLTVSSGGG |
| | GSEVQLVESGGGLVQPGRSL |
| | RLSCAASGFTFDDYTMHWVR |
| | QAPGKGLEWVSGISWNSGSI |
| | GYADSVKGRFTISRDNAKKS |
| | LYLQMNSLRAEDTALYYCAK |
| | DNSGYGHYYYGMDVWGQGTT |
| | VTVASGGGGSGGGGSGGGGS |
| | EIVMTQSPATLSVSPGERAT |
| | LSCRASQSVSSNLAWYQQKP |
| | GQAPRLLIYGASTRATGIPA |
| | RFSGSGSGTEFTLTISSLQS |
| | EDFAVYYCQHYINWPLTFGG |
| | GTKVEIKDKTHTCPPCPAPE |
| | LLGGPSVFLFPPKPKDTLMI |
| | SRTPEVTCVVVDVSHEDPEV |
| | KFNWYVDGVEVHNAKTKPRE |
| | EQYNSTYRVVSVLTVLHQDW |
| | LNGKEYKCKVSNKALPAPIE |
| | KTISKAKGQPREPQVYTLPP |
| | SRDELTKNQVSLTCLVKGFY |
| | PSDIAVEWESNGQPENNYKT |
| | TPPVLDSDGSFFLYSKLTVD |
| | KSRWQQGNVFSCSVMHEALH |
| | NHYTQKSLSLSPGK |
| | (SEQ ID NO: 98) |
| BITE 6 | MGWSCIILFLVATATGVHSQ |
| | AVVTQESALTTSPGETVTLT |
| | CRSSTGAVTTGNYPNWVQEK |
| | PDHLFTGLIAGTNNRAPGVP |
| | ARFSGSLIGDKAALTITGAQ |
| | TEDEAIYFCALWFSSHWVFG |
| | GGTKLTVLGGGGSGGGGSGG |
| | GGSEVQLQQSGPVLVKPGAS |
| | VKISCKASGYSFTGYYMHWV |
| | RQSLVKRLEWIGRINPYTGV |
| | PSYKHNFKDKASLTVDKSSS |
| | TAYMELHSLTSEDSAVYYCA |
| | RELGGYWGQGTTLTVSSGGG |
| | GSGGGGSAVQLVESGGGLVQ |
| | PGNSLRLSCAASGFTFRSFG |
| | MSWVRQAPGKEPEWVSSISG |
| | SGSDTLYADSVKGRFTISRD |
| | NAKTTLYLQMNSLKPEDTAV |
| | YYCTIGGSLSRSSQGTQVTV |
| | SSGGGGSGGGGSEVQLVESG |
| | GGLVQPGRSLRLSCAASGFT |
| | FDDYTMHWVRQAPGKGLEWV |
| | SGISWNSGSIGYADSVKGRF |
| | TISRDNAKKSLYLQMNSLRA |
| | EDTALYYCAKDNSGYGHYYY |
| | GMDVWGQGTTVTVASGGGGS |
| | GGGGSGGGGSEIVMTQSPAT |
| | LSVSPGERATLSCRASQSVS |
| | SNLAWYQQKPGQAPRLLIYG |
| | ASTRATGIPARFSGSGSGTE |
| | FTLTISSLQSEDFAVYYCQH |
| | YINWPLTFGGGTKVEIKHHH |
| | HHH |
| | (SEQ ID NO: 99) |
| BITE 5 | MGWSCIILFLVATATGVHSE |
| Heavy | VQLQQSGPVLVKPGASVKIS |
| Chain 1 | CKASGYSFTGYYMHWVRQSL |
| | VKRLEWIGRINPYTGVPSYK |
| | HNFKDKASLTVDKSSSTAYM |
| | ELHSLTSEDSAVYYCARELG |
| | GYWGQGTTLTVSSASTKGPS |
| | VFPLAPSSKSTSGGTAALGC |
| | LVKDYFPEPVTVSWNSGALT |
| | SGVHTFPAVLQSSGLYSLSS |
| | VVTVPSSSLGTQTYICNVNH |
| | KPSNTKVDKKVEPKSCDKTH |
| | TCPPCPAPELLGGPSVFLFP |
| | PKPKDTLMISRTPEVTCVVV |
| | DVSHEDPEVKFNWYVDGVEV |
| | HNAKTKPREEQYNSTYRVVS |
| | VLTVLHQDWLNGKEYKCKVS |
| | NKALPAPIEKTISKAKGQPR |
| | EPQVYTLPPSRDELTKNQVS |
| | LWCLVKGFYPSDIAVEWESN |
| | GQPENNYKTTPPVLDSDGSF |
| | FLYSKLTVDKSRWQQGNVFS |
| | CSVMHEALHNHYTQKSLSLS |
| | PGK |
| | (SEQ ID NO: 100) |
| BITE 5 | MGWSCIILFLVATATGVHSQ |
| Light | AVVTQESALTTSPGETVTLT |
| Chain 1 | CRSSTGAVTTGNYPNWVQEK |
| | PDHLFTGLIAGTNNRAPGVP |
| | ARFSGSLIGDKAALTITGAQ |
| | TEDEAIYFCALWFSSHWVFG |
| | GGTKLTVLGQPKAAPSVTLF |
| | PPSSEELQANKATLVCLISD |
| | FYPGAVTVAWKADSSPVKAG |
| | VETTTPSKQSNNKYAASSYL |
| | SLTPEQWKSHRSYSCQVTHE |
| | GSTVEKTVAPTECS |
| | (SEQ ID NO: 101) |
| BITE 5 | MGWSCIILFLVATATGVHSE |
| Heavy | VQLVESGGGLVQPGRSLRLS |
| Chain 2 | CAASGFTFDDYTMHWVRQAP |
| | GKGLEWVSGISWNSGSIGYA |
| | DSVKGRFTISRDNAKKSLYL |
| | QMNSLRAEDTALYYCAKDNS |
| | GYGHYYYGMDVWGQGTTVTV |
| | ASRTVAAPSVFIFPPSDEQL |
| | KSGTASVVCLLNNFYPREAK |
| | VQWKVDNALQSGNSQESVTE |
| | QDSKDSTYSLSSTLTLSKAD |
| | YEKHKVYACEVTHQGLSSPV |
| | TKSFNRGECDKTHTCPPCPA |
| | PELLGGPSVFLFPPKPKDTL |
| | MISRTPEVTCVVVDVSHEDP |
| | EVKFNWYVDGVEVHNAKTKP |
| | REEQYNSTYRVVSVLTVLHQ |
| | DWLNGKEYKCKVSNKALPAP |
| | IEKTISKAKGQPREPQVYTL |
| | PPSRDELTKNQVSLSCAVKG |
| | FYPSDIAVEWESNGQPENNY |
| | KTTPPVLDSDGSFFLVSKLT |
| | VDKSRWQQGNVFSCSVMHEA |
| | LHNHYTQKSLSLSPGK |
| | (SEQ ID NO: 102) |
| BITE 5 | MGWSCIILFLVATATGVHSE |
| Light | IVMTQSPATLSVSPGERATL |
| Chain 2 | SCRASQSVSSNLAWYQQKPG |
| | QAPRLLIYGASTRATGIPAR |
| | FSGSGSGTEFTLTISSLQSE |
| | DFAVYYCQHYINWPLTFGGG |
| | TKVEIKASTKGPSVFPLAPS |
| | SKSTSGGTAALGCLVKDYFP |
| | EPVTVSWNSGALTSGVHTFP |
| | AVLQSSGLYSLSSVVTVPSS |
| | SLGTQTYICNVNHKPSNTKV |
| | DKKVEPKSC |
| | (SEQ ID NO: 103) |

The Fc region of BiTE 5 included knobs-into-holes (KiH) mutations. KiH is a well-validated heterodimerization technology for the third constant domain of an antibody. KiH engineering facilitates the construction of a bispecific antibody by making complementary mutations in the CH3 domain of each heavy chain (HC).

For comparison. BiTEs comprising humanized anti-mesothelin SS1 (huSS1) Fv or anti-CD19 Fv were also prepared. The structures of these are shown in FIGS. 13E-13F. The amino acid sequences are shown in Table 19.

TABLE 19

| Name | Amino Acid Sequence |
|---|---|
| BITE 7 | MGWSCIILFLVATATGVHSQ |
| Heavy Chain 1 | VQLVQSGAEVKKPGASVKVS |
| | CKASGYSFTGYTMNWVRQAP |
| | GQGLEWMGLITPYNGASSYN |
| | QKFRGKATMTVDTSTSTVYM |
| | ELSSLRSEDTAVYYCARGGY |
| | DGRGFDYWGQGTLVTVSSAS |
| | TKGPSVFPLAPSSKSTSGGT |
| | AALGCLVKDYFPEPVTVSWN |
| | SGALTSGVHTFPAVLQSSGL |
| | YSLSSVVTVPSSSLGTQTYI |
| | CNVNHKPSNTKVDKKVEPKS |
| | CDKTHTCPPCPAPELLGGPS |
| | VFLFPPKPKDTLMISRTPEV |
| | TCVVVDVSHEDPEVKFNWYV |
| | DGVEVHNAKTKPREEQYNST |
| | YRVVSVLTVLHQDWLNGKEY |
| | KCKVSNKALPAPIEKTISKA |
| | KGQPREPQVYTLPPSRDELT |
| | KNQVSLWCLVKGFYPSDIAV |
| | EWESNGQPENNYKTTPPVLD |
| | SDGSFFLYSKLTVDKSRWQQ |
| | GNVFSCSVMHEALHNHYTQK |
| | SLSLSPGK |
| | (SEQ ID NO: 104) |
| BITE 7 | MGWSCIILFLVATATGVHSD |
| Light Chain 1 | IQMTQSPSSLSASVGDRVTI |
| | TCSASSSVSYMHWYQQKSGK |
| | APKLLIYDTSKLASGVPSRF |
| | SGSGSGTDFTLTISSLQPED |
| | FATYYCQQWSKHPLTFGQGT |
| | KLEIKRTVAAPSVFIFPPSD |
| | EQLKSGTASVVCLLNNFYPR |
| | EAKVQWKVDNALQSGNSQES |
| | VTEQDSKDSTYSLSSTLTLS |
| | KADYEKHKVYACEVTHQGLS |
| | SPVTKSFNRGEC |
| | (SEQ ID NO: 105) |
| BITE 7 | MGWSCIILFLVATATGVHSE |
| Heavy Chain 2 | VQLVESGGGLVQPGRSLRLS |
| | CAASGFTFDDYTMHWVRQAP |
| | GKGLEWVSGISWNSGSIGYA |
| | DSVKGRFTISRDNAKKSLYL |
| | QMNSLRAEDTALYYCAKDNS |
| | GYGHYYYGMDVWGQGTTVTV |
| | ASRTVAAPSVFIFPPSDEQL |
| | KSGTASVVCLLNNFYPREAK |
| | VQWKVDNALQSGNSQESVTE |
| | QDSKDSTYSLSSTLTLSKAD |
| | YEKHKVYACEVTHQGLSSPV |
| | TKSFNRGECDKTHTCPPCPA |
| | PELLGGPSVFLFPPKPKDTL |
| | MISRTPEVTCVVVDVSHEDP |
| | EVKFNWYVDGVEVHNAKTKP |
| | REEQYNSTYRVVSVLTVLHQ |
| | DWLNGKEYKCKVSNKALPAP |
| | IEKTISKAKGQPREPQVYTL |
| | PPSRDELTKNQVSLSCAVKG |
| | FYPSDIAVEWESNGQPENNY |
| | KTTPPVLDSDGSFFLVSKLT |
| | VDKSRWQQGNVFSCSVMHEA |
| | LHNHYTQKSLSLSPGK |
| | (SEQ ID NO: 102) |
| BITE 7 | MGWSCIILFLVATATGVHSE |
| Light Chain 2 | IVMTQSPATLSVSPGERATL |
| | SCRASQSVSSNLAWYQQKPG |
| | QAPRLLIYGASTRATGIPAR |
| | FSGSGSGTEFTLTISSLQSE |
| | DFAVYYCQHYINWPLTFGGG |
| | TKVEIKASTKGPSVFPLAPS |
| | SKSTSGGTAALGCLVKDYFP |
| | EPVTVSWNSGALTSGVHTFP |
| | AVLQSSGLYSLSSVVTVPSS |
| | SLGTQTYICNVNHKPSNTKV |
| | DKKVEPKSC |
| | (SEQ ID NO: 103) |
| BITE 8 | MGWSCIILFLVATATGVHSQ |
| Heavy Chain 1 | VQLQQSGAELVRPGSSVKIS |
| | CKASGYAFSSYWMNWVKQRP |
| | GQGLEWIGQIWPGDGDTNYN |
| | GKFKGKATLTADESSSTAYM |
| | QLSSLASEDSAVYFCARRET |
| | TTVGRYYYAMDYWGQGTTVT |
| | VSSASTKGPSVFPLAPSSKS |
| | TSGGTAALGCLVKDYFPEPV |
| | TVSWNSGALTSGVHTFPAVL |
| | QSSGLYSLSSVVTVPSSSLG |
| | TQTYICNVNHKPSNTKVDKK |
| | VEPKSCDKTHTCPPCPAPEL |
| | LGGPSVFLFPPKPKDTLMIS |
| | RTPEVTCVVVDVSHEDPEVK |
| | FNWYVDGVEVHNAKTKPREE |
| | QYNSTYRVVSVLTVLHQDWL |
| | NGKEYKCKVSNKALPAPIEK |
| | TISKAKGQPREPQVYTLPPS |
| | RDELTKNQVSLWCLVKGFYP |
| | SDIAVEWESNGQPENNYKTT |
| | PPVLDSDGSFFLYSKLTVDK |
| | SRWQQGNVFSCSVMHEALHN |
| | HYTQKSLSLSPGK |
| | (SEQ ID NO: 108) |
| BITE 8 | MGWSCIILFLVATATGVHSD |
| Light Chain 1 | IQLTQSPASLAVSLGQRATI |
| | SCKASQSVDYDGDSYLNWYQ |
| | QIPGQPPKLLIYDASNLVSG |
| | IPPRFSGSGSGTDFTLNIHP |
| | VEKVDAATYHCQQSTEDPWT |
| | FGGGTKLEIKRTVAAPSVFI |
| | FPPSDEQLKSGTASVVCLLN |
| | NFYPREAKVQWKVDNALQSG |
| | NSQESVTEQDSKDSTYSLSS |
| | TLTLSKADYEKHKVYACEVT |
| | HQGLSSPVTKSFNRGEC |
| | (SEQ ID NO: 109) |
| BITE 8 | MGWSCIILFLVATATGVHSE |
| Heavy Chain 2 | VQLVESGGGLVQPGRSLRLS |
| | CAASGFTFDDYTMHWVRQAP |
| | GKGLEWVSGISWNSGSIGYA |
| | DSVKGRFTISRDNAKKSLYL |
| | QMNSLRAEDTALYYCAKDNS |
| | GYGHYYYGMDVWGQGTTVTV |
| | ASRTVAAPSVFIFPPSDEQL |
| | KSGTASVVCLLNNFYPREAK |
| | VQWKVDNALQSGNSQESVTE |
| | QDSKDSTYSLSSTLTLSKAD |
| | YEKHKVYACEVTHQGLSSPV |
| | TKSFNRGECDKTHTCPPCPA |
| | PELLGGPSVFLFPPKPKDTL |
| | MISRTPEVTCVVVDVSHEDP |
| | EVKFNWYVDGVEVHNAKTKP |
| | REEQYNSTYRVVSVLTVLHQ |
| | DWLNGKEYKCKVSNKALPAP |
| | IEKTISKAKGQPREPQVYTL |
| | PPSRDELTKNQVSLSCAVKG |
| | FYPSDIAVEWESNGQPENNY |
| | KTTPPVLDSDGSFFLVSKLT |
| | VDKSRWQQGNVFSCSVMHEA |
| | LHNHYTQKSLSLSPGK |
| | (SEQ ID NO: 102) |
| BITE 8 | MGWSCIILFLVATATGVHSE |
| Light Chain 2 | IVMTQSPATLSVSPGERATL |
| | SCRASQSVSSNLAWYQQKPG |
| | QAPRLLIYGASTRATGIPAR |
| | FSGSGSGTEFTLTISSLQSE |
| | DFAVYYCQHYINWPLTFGGG |
| | TKVEIKASTKGPSVFPLAPS |
| | SKSTSGGTAALGCLVKDYFP |
| | EPVTVSWNSGALTSGVHTFP |
| | AVLQSSGLYSLSSVVTVPSS |
| | SLGTQTYICNVNHKPSNTKV |
| | DKKVEPKSC |
| | (SEQ ID NO: 103) |

### EXAMPLE 16

This example demonstrates that BiTE 5 kills multiple different mesothelin-expressing cancer cell lines.

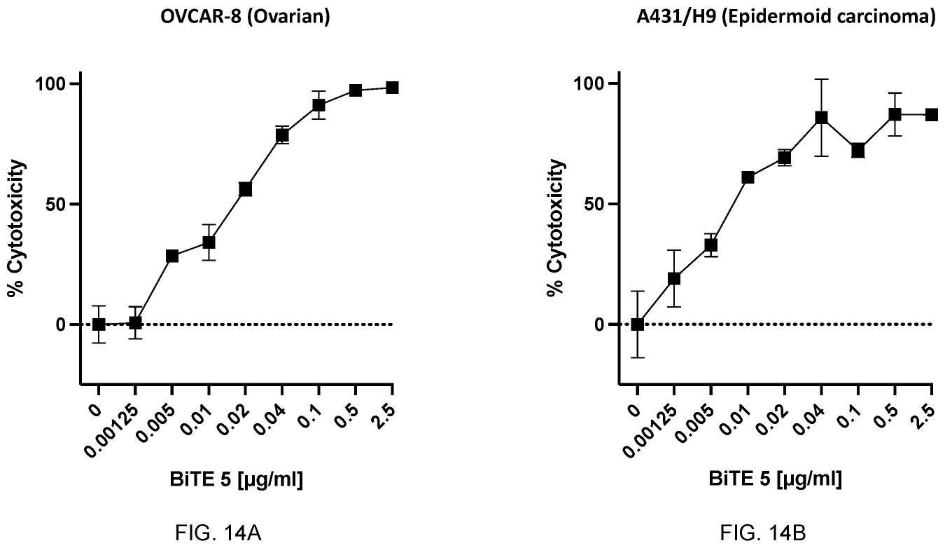

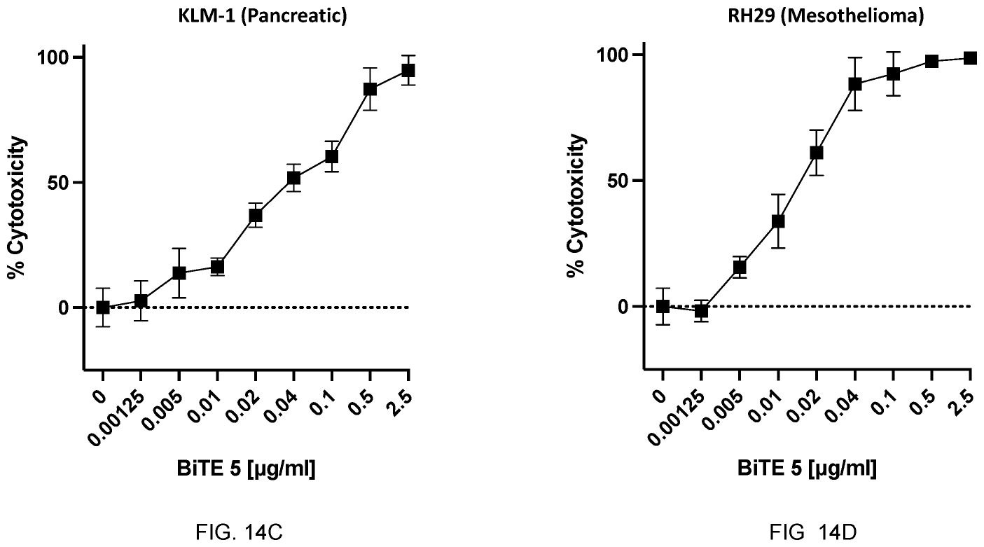

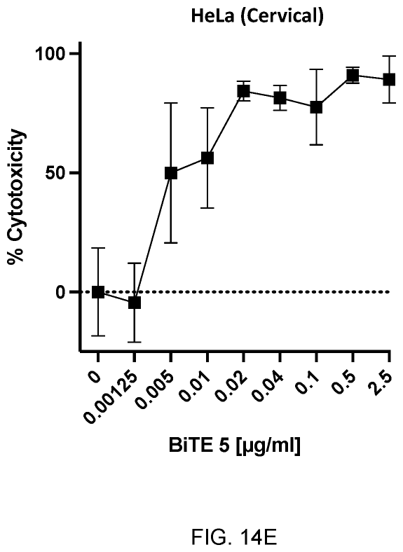

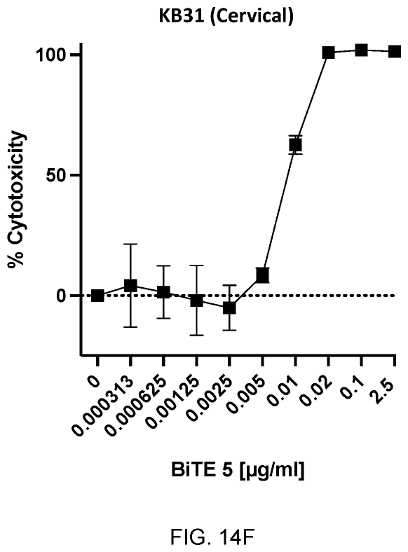

PBMCs and various dosages of BiTE 5 of Example 15 were independently co-cultured with target mesothelin-expressing cancer cell lines OVCAR-8 (ovarian), A431/H9 (epidermoid carcinoma), KLM-1 (pancreatic), RH29 (mesothelioma), or HeLa (cervical). The cancer cell lines also expressed luciferase. Cancer cells were seeded in a 96-well plate (2,000 cells/well) on Day 0. Media was removed, and human donor PBMCs (10:1 effector to target (E/T) ratio) and various dosages of BiTE 5 of Example 15 (4 replicates per dose) were added in fresh media on Day 1. Media was removed, and viability was assessed on Day 4 with the luciferase assay. The results are shown in FIGS. 14A-14E. BiTE 5 killed the mesothelin-expressing cancer cell lines.

T-cells and various dosages of BiTE 5 of Example 15 were also co-cultured with the mesothelin-expressing cancer cell line KB31 (cervical). The KB31 cells were seeded in a 96-well plate (3,000 cells/well) on Day 0. The media was removed, and human donor T-cells (isolated from PBMCs: 10:1 E/T ratio) and various dosages of BITE 5 of Example 15 (2 replicates per dose) were added in fresh media on Day 1. The cells were washed with PBS on Day 4. Viability was assessed with the WST-8 assay. The results are shown in FIG. 14F. BiTE 5 killed the mesothelin-expressing cancer cell line KB31.

### EXAMPLE 17

This example demonstrates the cytotoxic activity of BiTE 5 based on mesothelin expression in cancer cell lines.

Luciferase-expressing target cells were seeded in a 96-well plate (2,000 cells/well) on Day 0. The target cells were mesothelin-positive pancreatic cancer cell line KLM-1 or mesothelin-negative knock-out cell line KLM-1 KO #2. The media was removed. Human donor PBMCs (30:1 E/T ratio) and various dosages of BiTE 5 of Example 15 (four replicates per dose) were added in fresh media on Day 1. Media was removed and viability was assessed on Day 4 with the luciferase assay. The results are shown in FIGS. 15A-15B. The level of cytotoxic activity provided by BiTE 5 depended on mesothelin expression of the target cell.

### EXAMPLE 18

This example demonstrates that BiTE 5 effectively inhibits the growth of KB31 tumor in NSG mice.

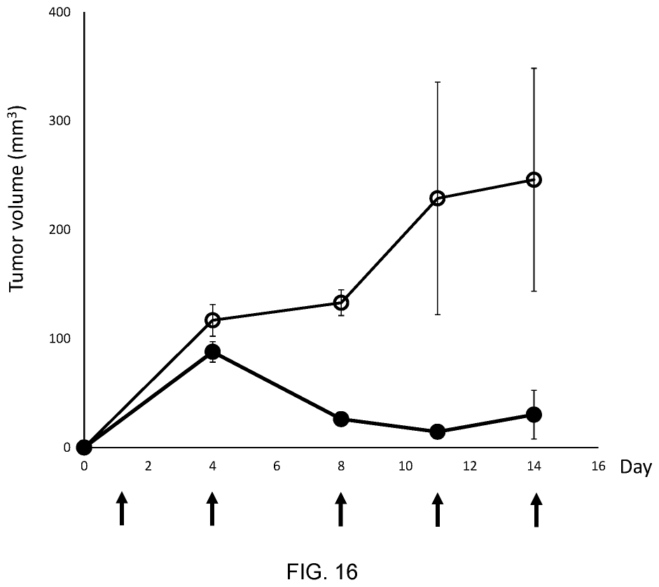

KB31 and T cells from a healthy donor were co-implanted subcutaneously into NSG mice on Day 0 at an E: T of 2:1. Mice were treated with BiTE 5 of Example 15 (2.5 mg/kg) or PBS on Days 1, 4, 8, 11, and 14. Tumor volume was measured up to Day 14. The results are shown in FIG. 16. BiTE 5 effectively inhibited the growth of KB31 tumor in NSG mice.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred aspects of this invention are described herein, including the best mode known to the inventors for carrying out the invention. Variations of those preferred aspects may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A polypeptide which specifically binds to human mesothelin₅₈₂₋₅₉₈ (IPNGYLVLDLSMQEALS) (SEQ ID NO: 1) and which comprises:
(A) the light chain complementary determining region (VL CDR) 1 amino acid sequence of SEQ ID NO: 17;
the VL CDR2 amino acid sequence of SEQ ID NO: 19;
the VL CDR3 amino acid sequence of SEQ ID NO: 21;
the heavy chain complementary determining region (VH CDR) 1 amino acid sequence of SEQ ID NO: 24;
the VH CDR2 amino acid sequence of SEQ ID NO: 26; and
the VH CDR3 amino acid sequence of SEQ ID NO: 28;
(B) the VL CDR1 amino acid sequence of SEQ ID NO: 17;
the VL CDR2 amino acid sequence of SEQ ID NO: 19;
the VL CDR3 amino acid sequence of SEQ ID NO: 21;
the VH CDR1 amino acid sequence of SEQ ID NO: 31;
the VH CDR2 amino acid sequence of SEQ ID NO: 33; and
the VH CDR3 amino acid sequence of SEQ ID NO: 35;
(C) the VL CDR1 amino acid sequence of SEQ ID NO: 38;
the VL CDR2 amino acid sequence of SEQ ID NO: 40;
the VL CDR3 amino acid sequence of SEQ ID NO: 42;
the VH CDR1 amino acid sequence of SEQ ID NO: 24;
the VH CDR2 amino acid sequence of SEQ ID NO: 26; and
the VH CDR3 amino acid sequence of SEQ ID NO: 28; or (D) the VL CDR1 amino acid sequence of SEQ ID NO: 38;
the VL CDR2 amino acid sequence of SEQ ID NO: 40;
the VL CDR3 amino acid sequence of SEQ ID NO: 42;
the VH CDR1 amino acid sequence of SEQ ID NO: 31;
the VH CDR2 amino acid sequence of SEQ ID NO: 33; and
the VH CDR3 amino acid sequence of SEQ ID NO: 35.

2. The polypeptide of claim 1 comprising:
(A) the heavy chain variable region (VH) amino acid sequence of SEQ ID NO: 46 and the light chain variable region (VL) amino acid sequence of SEQ ID NO: 48;
(B) the VH amino acid sequence of SEQ ID NO: 47 and the VL amino acid sequence of SEQ ID NO: 48;
(C) the VH amino acid sequence of SEQ ID NO: 46 and the VL amino acid sequence of SEQ ID NO: 49; or
(D) the VH amino acid sequence of SEQ ID NO: 47 and the VL amino acid sequence of SEQ ID NO: 49.

3. The polypeptide of claim 1 comprising, in order from the amino terminus to the carboxyl terminus, the VL CDR1 amino acid sequence, the VL CDR2 amino acid sequence, the VL CDR3 amino acid sequence, the VH CDR1 amino acid sequence, the VH CDR2 amino acid sequence, and the VH CDR3 amino acid sequence.

4. A protein which specifically binds to human mesothelin₅₈₂₋₅₉₈ (IPNGYLVLDLSMQEALS) (SEQ ID NO: 1) and which comprises:
(A) a first polypeptide chain comprising the light chain complementary determining region (VL CDR) 1 amino acid sequence of SEQ ID NO: 17; the VL CDR2 amino acid sequence of SEQ ID NO: 19; and the VL CDR3 amino acid sequence of SEQ ID NO: 21; and
a second polypeptide chain comprising the heavy chain complementary determining region (VH CDR) 1 amino acid sequence of SEQ ID NO: 24; the VH CDR2 amino acid sequence of SEQ ID NO: 26; and the VH CDR3 amino acid sequence of SEQ ID NO: 28;
(B) a first polypeptide chain comprising the VL CDR1 amino acid sequence of SEQ ID NO: 17; the VL CDR2 amino acid sequence of SEQ ID NO: 19; and the VL CDR3 amino acid sequence of SEQ ID NO: 21; and
a second polypeptide chain comprising the VH CDR1 amino acid sequence of SEQ ID NO: 31; the VH CDR2 amino acid sequence of SEQ ID NO: 33; and the VH CDR3 amino acid sequence of SEQ ID NO: 35;
(C) a first polypeptide chain comprising the VL CDR1 amino acid sequence of SEQ ID NO: 38; the VL CDR2 amino acid sequence of SEQ ID NO: 40; and the VL CDR3 amino acid sequence of SEQ ID NO: 42; and
a second polypeptide chain comprising the VH CDR1 amino acid sequence of SEQ ID NO: 24; the VH CDR2 amino acid sequence of SEQ ID NO: 26; and the VH CDR3 amino acid sequence of SEQ ID NO: 28; or
(D) a first polypeptide chain comprising the VL CDR1 amino acid sequence of SEQ ID NO: 38; the VL CDR2 amino acid sequence of SEQ ID NO: 40; and the VL CDR3 amino acid sequence of SEQ ID NO: 42; and
a second polypeptide chain comprising the VH CDR1 amino acid sequence of SEQ ID NO: 31; the VH CDR2 amino acid sequence of SEQ ID NO: 33; and the VH CDR3 amino acid sequence of SEQ ID NO: 35.

5. The protein of claim 4, wherein:
(A) the first polypeptide chain comprises the light chain variable region (VL) amino acid sequence of SEQ ID NO: 48 and the second polypeptide chain comprises the heavy chain variable region (VH) amino acid sequence of SEQ ID NO: 46;
(B) the first polypeptide chain comprises the VL amino acid sequence of SEQ ID NO: 48 and the second polypeptide chain comprises the VH amino acid sequence of SEQ ID NO: 47; (C) the first polypeptide chain comprises the VL amino acid sequence of SEQ ID NO: 49 and the second polypeptide chain comprises the VH amino acid sequence of SEQ ID NO: 46; or
(D) the first polypeptide chain comprises the VL amino acid sequence of SEQ ID NO: 49 and the second polypeptide chain comprises the VH amino acid sequence of SEQ ID NO: 47.

6. An anti-mesothelin binding moiety comprising the polypeptide of claim 1, wherein the anti-mesothelin binding moiety is an antibody, Fab fragment (Fab), F(ab′)₂ fragment, diabody, triabody, tetrabody, multispecific antibody, single-chain variable region fragment (scFv), or disulfide-stabilized variable region fragment (dsFv).

7. The anti-mesothelin binding moiety of claim 6, wherein the anti-mesothelin binding moiety further comprises an agent which specifically binds to an immune cell.

8. The anti-mesothelin binding moiety of claim 7, wherein the agent which specifically binds to the immune cell is a T cell engager or an NK cell engager,
optionally wherein the agent which specifically binds to an immune cell is a bispecific T cell engager, a bispecific NK cell engager, a trispecific T cell engager, or a trispecific NK cell engager.

9. The anti-mesothelin binding moiety of claim 6, wherein the anti-mesothelin binding moiety is an scFv, and the scFv comprises the amino acid sequence of any one of SEQ ID NOs: 50-51 and 58-61.

10. The anti-mesothelin binding moiety of claim 8, wherein the anti-mesothelin binding moiety is a bi-specific T-cell engager (BiTE).

11. The anti-mesothelin binding moiety of claim 10, wherein the BiTE comprises (i) the amino acid sequence of SEQ ID NO: 97, (ii) the amino acid sequence of SEQ ID NO: 98, (iii) the amino acid sequence of SEQ ID NO: 99, or (iv) the amino acid sequences of all of SEQ ID NO: 100, SEQ ID NO: 101, SEQ ID NO: 102, and SEQ ID NO: 103.

12. A conjugate comprising (a) the polypeptide of claim 1, conjugated or fused to (b) an effector molecule, wherein the effector molecule is a drug, toxin, label, small molecule, or an antibody.

13. The conjugate according to claim 12, wherein the effector molecule is Pseudomonas exotoxin A (PE) or a variant thereof.

14. A chimeric antigen receptor (CAR) comprising an antigen binding domain, a transmembrane (TM) domain, and an intracellular T cell signaling domain, wherein the antigen binding domain has antigen specificity for human mesothelin₅₈₂₋₅₉₈ (IPNGYLVLDLSMQEALS) (SEQ ID NO: 1), and wherein the antigen binding domain comprises:
(A) the light chain complementary determining region (VL CDR) 1 amino acid sequence of SEQ ID NO: 17; the VL CDR2 amino acid sequence of SEQ ID NO: 19; the VL CDR3 amino acid sequence of SEQ ID NO: 21; the heavy chain complementary determining region (VH CDR) 1 amino acid sequence of SEQ ID NO: 24; the VH CDR2 amino acid sequence of SEQ ID NO: 26; and the VH CDR3 amino acid sequence of SEQ ID NO: 28;
(B) the VL CDR1 amino acid sequence of SEQ ID NO: 17; the VL CDR2 amino acid sequence of SEQ ID NO: 19; the VL CDR3 amino acid sequence of SEQ ID NO: 21; the VH CDR1 amino acid sequence of SEQ ID NO: 31; the VH CDR2 amino acid sequence of SEQ ID NO: 33; and the VH CDR3 amino acid sequence of SEQ ID NO: 35;
(C) the VL CDR1 amino acid sequence of SEQ ID NO: 38; the VL CDR2 amino acid sequence of SEQ ID NO: 40; the VL CDR3 amino acid sequence of SEQ ID NO: 42; the VH CDR1 amino acid sequence of SEQ ID NO: 24; the VH CDR2 amino acid sequence of SEQ ID NO: 26; and the VH CDR3 amino acid sequence of SEQ ID NO: 28;
(D) the VL CDR1 amino acid sequence of SEQ ID NO: 38; the VL CDR2 amino acid sequence of SEQ ID NO: 40; the VL CDR3 amino acid sequence of SEQ ID NO: 42; the VH CDR1 amino acid sequence of SEQ ID NO: 31; the VH CDR2 amino acid sequence of SEQ ID NO: 33; and the VH CDR3 amino acid sequence of SEQ ID NO: 35; or
(E) the VL CDR1 amino acid sequence of SEQ ID NO: 3; the VL CDR2 amino acid sequence of SEQ ID NO: 5; the VL CDR3 amino acid sequence of SEQ ID NO: 7; the VH CDR1 amino acid sequence of SEQ ID NO: 10; the VH CDR2 amino acid sequence of SEQ ID NO: 12; and the VH CDR3 amino acid sequence of SEQ ID NO: 14.

15. The CAR of claim 14 comprising:
(A) the heavy chain variable region (VH) amino acid sequence of SEQ ID NO: 46 and the light chain variable region (VL) amino acid sequence of SEQ ID NO: 48;
(B) the VH amino acid sequence of SEQ ID NO: 47 and the VL amino acid sequence of SEQ ID NO: 48;
(C) the VH amino acid sequence of SEQ ID NO: 46 and the VL amino acid sequence of SEQ ID NO: 49;
(D) the VH amino acid sequence of SEQ ID NO: 47 and the VL amino acid sequence of SEQ ID NO: 49; or (E) the VH amino acid sequence of SEQ ID NO: 44 and the VL amino acid sequence of SEQ ID NO: 45.

16. The CAR of claim 14 comprising, in order from the amino terminus to the carboxyl terminus, the VL CDR1 amino acid sequence, the VL CDR2 amino acid sequence, the VL CDR3 amino acid sequence, the VH CDR1 amino acid sequence, the VH CDR2 amino acid sequence, and the VH CDR3 amino acid sequence.

17. The CAR of claim 14, wherein the antigen binding domain comprises the amino acid sequence of any one of SEQ ID NOs: 50-51 and 58-61.

18. The CAR of claim 14, wherein the intracellular T cell signaling domain comprises the intracellular T cell signaling domain of any one of the following proteins: a CD3-zeta protein, a CD27 protein, a CD28 protein, a CD40 protein, a FcRγ protein, an inducible T-cell costimulatory protein (ICOS), a killer cell immunoglobulin-like receptor 2DS2 protein (KIR2DS2), a MYD88 protein, a OX40 protein, a 4-1BB protein, or any combination of the foregoing.

19. The CAR of claim 14, wherein the transmembrane domain comprises any one of the following: a CD3 zeta transmembrane domain, a CD4 transmembrane domain, a CD8 transmembrane domain, a CD28transmembrane domain, a ICOS transmembrane domain, or any combination of the foregoing.

20. The CAR of claim 14, further comprising a hinge domain of any one of the following proteins: a CD8 protein, a CD28 protein, a IgG1 protein, or a IgG4 protein.

21. The CAR of claim 14 comprising the amino acid sequence of any one of SEQ ID NOs: 70-77, 86-87, and 90-91.

22. A bispecific, biparatopic CAR comprising the CAR of claim 14, wherein the antigen binding domain of the CAR of claim 14 is a first antigen binding domain, and the bispecific, biparatopic CAR further comprises a second antigen binding domain having antigen specificity for a human mesothelin epitope other than human mesothelin₅₈₂₋₅₉₈ (IPNGYLVLDLSMQEALS) (SEQ ID NO: 1).

23. A nucleic acid comprising a nucleotide sequence encoding the polypeptide of claim 1.

24. A nucleic acid comprising a nucleotide sequence encoding a chimeric antigen receptor (CAR) construct comprising:
(a) a first CAR, wherein the first CAR is the CAR of claim 14;
(b) a second CAR comprising
a second antigen binding domain,
a second transmembrane domain, and
a second intracellular T cell signaling domain; and
(c) a cleavage sequence:
wherein the cleavage sequence is positioned between the first and second CARs, and
wherein the second CAR specifically binds to a human mesothelin epitope other than human mesothelin₅₈₂₋₅₉₈ (IPNGYLVLDLSMQEALS) (SEQ ID NO: 1).

25. The nucleic acid of claim 24, wherein the antigen binding domain of the second CAR comprises the antigen binding domain of mAb YP218 or humanized mAb YP218.

26. The nucleic acid of claim 24, wherein the nucleic acid comprises a nucleotide sequence encoding the amino acid sequence of SEQ ID NO: 93.

27. A nucleic acid comprising a nucleotide sequence encoding:
(a) the polypeptide of claim 1;
(b) a CAR comprising
an antigen binding domain,
a transmembrane domain, and
an intracellular T cell signaling domain; and
(c) a cleavage sequence;
wherein the cleavage sequence is positioned between the polypeptide of (a) and the CAR of (b), and
wherein the CAR of (b) specifically binds to a human mesothelin epitope other than human mesothelin₅₈₂₋₅₉₈ (IPNGYLVLDLSMQEALS) (SEQ ID NO: 1).

28. The nucleic acid of claim 27, wherein the polypeptide comprises the amino acid sequence of any one of SEQ ID NOs: 50-51 and 58-61.

29. The nucleic acid of claim 27, wherein the antigen binding domain of the CAR of (b) comprises the antigen binding domain of mAb YP218 or humanized mAb YP218.

30. The nucleic acid of claim 27, wherein the nucleic acid comprises a nucleotide sequence encoding the amino acid sequence of SEQ ID NO: 96.

31. A recombinant expression vector comprising the nucleic acid of claim 23.

32. An isolated host cell comprising the recombinant expression vector of claim 31.

33. A population of cells comprising at least one host cell of claim 32.

34. A polypeptide encoded by the nucleic acid of claim 24.

35. A pharmaceutical composition comprising the polypeptide of claim 1 and a pharmaceutically acceptable carrier.

36. (canceled)

37. A method of detecting the presence of cancer in a mammal, the method comprising:
(a) contacting a sample comprising one or more cells from the mammal with the polypeptide of claim 1, thereby forming a complex, and
(b) detecting the complex, wherein detection of the complex is indicative of the presence of cancer.

38-45. (canceled)

46. A method of reducing mesothelin shed from cell membranes, the method comprising administering to cells the polypeptide of claim 1 in an amount effective to reduce mesothelin shed from the cell membranes of the cells.

47. A method of treating or preventing cancer in a mammal, the method comprising administering to the mammal the polypeptide of claim 1 in an amount effective to treat or prevent cancer in the mammal.

48. A method of treating or preventing cancer in a mammal, the method comprising co-administering to the mammal:
(a) the polypeptide of claim 1 and
(b) a further agent that specifically binds to a human mesothelin epitope other than human mesothelin₅₈₂₋₅₉₈ (IPNGYLVLDLSMQEALS) (SEQ ID NO: 1) and inhibits the growth of mesothelin-expressing cells.

49. The method of claim 48, wherein the method comprises co-administering (a) and (b) sequentially.

50. The method of claim 48, wherein the method comprises co-administering (a) and (b) simultaneously.

51. The method of claim 48, wherein the further agent is selected from one or more of the following: a polypeptide, a protein, a conjugate, and a CAR.

52. The method of claim 48, wherein the further agent is selected from one or more of the following: an antibody, Fab fragment (Fab), F (ab') 2 fragment, diabody, triabody, tetrabody, multispecific antibody, single-chain variable region fragment (scFv), and disulfide-stabilized variable region fragment (dsFv).

53. The method of claim 48, wherein the further agent is a conjugate comprising (a) an anti-mesothelin binding moiety conjugated or fused to (b) an effector molecule. wherein the effector molecule is a drug. toxin. label. small molecule. or an antibody

54. The method of claim 48. wherein the further agent comprises the antigen binding domain of mAb YP218 or humanized mAb YP218.


SEQUENCE LISTING (109 sequences)

SEQ ID NO: 1 - protein, 17 aa, Homo sapiens
IPNGYLVLDLSMQEALS

SEQ ID NO: 2 - protein, 22 aa, Mus musculus
QAVVTQESALTTSPGETVTLTC

SEQ ID NO: 3 - protein, 14 aa, Mus musculus
RSSTGAVTTGNYPN

SEQ ID NO: 4 - protein, 15 aa, Mus musculus
WVQEKPDHLFTGLIA

SEQ ID NO: 5 - protein, 7 aa, Mus musculus
GTNNRAP

SEQ ID NO: 6 - protein, 32 aa, Mus musculus
GVPARFSGSLIGDKAALTITGAQTEDEAIYFC

SEQ ID NO: 7 - protein, 9 aa, Mus musculus
ALWFSSHWV

SEQ ID NO: 8 - protein, 10 aa, Mus musculus
FGGGTKLTVL

SEQ ID NO: 9 - protein, 30 aa, Mus musculus
EVQLQQSGPVLVKPGASVKISCKASGYSFT

SEQ ID NO: 10 - protein, 5 aa, Mus musculus
GYYMH

SEQ ID NO: 11 - protein, 14 aa, Mus musculus
WVRQSLVKRLEWIG

SEQ ID NO: 12 - protein, 17 aa, Mus musculus
RINPYTGVPSYKHNFKD

SEQ ID NO: 13 - protein, 32 aa, Mus musculus
KASLTVDKSSSTAYMELHSLTSEDSAVYYCAR

SEQ ID NO: 14 - protein, 5 aa, Mus musculus
ELGGY

SEQ ID NO: 15 - protein, 11 aa, Mus musculus
WGQGTTLTVSS

SEQ ID NO: 16 - protein, 22 aa, synthetic construct
  REGION 1..22: note = Synthetic
QAVVTQEPSLTVSPGGTVTLTC

SEQ ID NO: 17 - protein, 14 aa, synthetic construct
  REGION 1..14: note = Synthetic
ASSTGAVTTGNYPN

SEQ ID NO: 18 - protein, 15 aa, synthetic construct
  REGION 1..15: note = Synthetic
WFQEKPGQAFRGLIA

SEQ ID NO: 19 - protein, 7 aa, synthetic construct
  REGION 1..7: note = Synthetic
GTNNRAP

SEQ ID NO: 20 - protein, 32 aa, synthetic construct
  REGION 1..32: note = Synthetic
WVPARFSGSLIGDKAALTLSGVQPEDEAEYFC

SEQ ID NO: 21 - protein, 9 aa, synthetic construct
  REGION 1..9: note = Synthetic
ALWFSSHWV

SEQ ID NO: 22 - protein, 10 aa, synthetic construct
  REGION 1..10: note = Synthetic
FGGGTKLTVL

SEQ ID NO: 23 - protein, 30 aa, synthetic construct
  REGION 1..30: note = Synthetic
EVQLVQSGAEVKKPGASVKVSCKASGYSFT

SEQ ID NO: 24 - protein, 5 aa, synthetic construct
  REGION 1..5: note = Synthetic
GYYMH

SEQ ID NO: 25 - protein, 14 aa, synthetic construct
  REGION 1..14: note = Synthetic
WVRQAPGQGLEWIG

SEQ ID NO: 26 - protein, 17 aa, synthetic construct
  REGION 1..17: note = Synthetic
RINPYTGVPSYKHKFQG

SEQ ID NO: 27 - protein, 32 aa, synthetic construct
  REGION 1..32: note = Synthetic
RVTLTVDKSTSTAYMELSSLRSEDTAVYYCAR

SEQ ID NO: 28 - protein, 5 aa, synthetic construct
  REGION 1..5: note = Synthetic
ELGGY

SEQ ID NO: 29 - protein, 11 aa, synthetic construct
  REGION 1..11: note = Synthetic
WGQGTTVTVSS

SEQ ID NO: 30 - protein, 30 aa, synthetic construct
  REGION 1..30: note = Synthetic
QVQLVQSGAEVKKPGASVKVSCKASGYSFT

SEQ ID NO: 31 - protein, 5 aa, synthetic construct
  REGION 1..5: note = Synthetic
GYYMH

SEQ ID NO: 32 - protein, 14 aa, synthetic construct
  REGION 1..14: note = Synthetic
WVRQAPGQGLEWMG

SEQ ID NO: 33 - protein, 17 aa, synthetic construct
  REGION 1..17: note = Synthetic
RINPYTGVPSYKHKFQG

SEQ ID NO: 34 - protein, 32 aa, synthetic construct
  REGION 1..32: note = Synthetic
RVTMTVDKSTSTAYMELSSLRSEDTAVYYCAR

SEQ ID NO: 35 - protein, 5 aa, synthetic construct
  REGION 1..5: note = Synthetic
ELGGY

SEQ ID NO: 36 - protein, 11 aa, synthetic construct
  REGION 1..11: note = Synthetic
WGQGTTVTVSS

SEQ ID NO: 37 - protein, 22 aa, synthetic construct
  REGION 1..22: note = Synthetic
QAVVTQEPSLTVSPGGTVTLTC

SEQ ID NO: 38 - protein, 14 aa, synthetic construct
  REGION 1..14: note = Synthetic
ASSTGAVTTGNYPN

SEQ ID NO: 39 - protein, 15 aa, synthetic construct
  REGION 1..15: note = Synthetic
WFQQKPGQAFRGLIA

SEQ ID NO: 40 - protein, 7 aa, synthetic construct
  REGION 1..7: note = Synthetic
GTNNKAS

SEQ ID NO: 41 - protein, 32 aa, synthetic construct
  REGION 1..32: note = Synthetic
WTPARFSGSLLGDKAALTLSGVQPEDEAEYYC

SEQ ID NO: 42 - protein, 9 aa, synthetic construct
  REGION 1..9: note = Synthetic
ALWFSSHWV

SEQ ID NO: 43 - protein, 10 aa, synthetic construct
  REGION 1..10: note = Synthetic
FGGGTKLTVL

SEQ ID NO: 44 - protein, 114 aa, Mus musculus
EVQLQQSGPVLVKPGASVKISCKASGYSFTGYYMHWVRQSLVKRLEWIGRINPYTGVPSYKHNFKDKASLTVDKSSSTAYMELHSLTSEDSAVYYCARELGGYWGQGTTLTVSS

SEQ ID NO: 45 - protein, 109 aa, Mus musculus
QAVVTQESALTTSPGETVTLTCRSSTGAVTTGNYPNWVQEKPDHLFTGLIAGTNNRAPGVPARFSGSLIGDKAALTITGAQTEDEAIYFCALWFSSHWVFGGGTKLTVL

SEQ ID NO: 46 - protein, 114 aa, synthetic construct
  REGION 1..114: note = Synthetic
EVQLVQSGAEVKKPGASVKVSCKASGYSFTGYYMHWVRQAPGQGLEWIGRINPYTGVPSYKHKFQGRVTLTVDKSTSTAYMELSSLRSEDTAVYYCARELGGYWGQGTTVTVSS

SEQ ID NO: 47 - protein, 114 aa, synthetic construct
  REGION 1..114: note = Synthetic
QVQLVQSGAEVKKPGASVKVSCKASGYSFTGYYMHWVRQAPGQGLEWMGRINPYTGVPSYKHKFQGRVTMTVDKSTSTAYMELSSLRSEDTAVYYCARELGGYWGQGTTVTVSS

SEQ ID NO: 48 - protein, 109 aa, synthetic construct
  REGION 1..109: note = Synthetic
QAVVTQEPSLTVSPGGTVTLTCASSTGAVTTGNYPNWFQEKPGQAFRGLIAGTNNRAPWVPARFSGSLIGDKAALTLSGVQPEDEAEYFCALWFSSHWVFGGGTKLTVL

SEQ ID NO: 49 - protein, 109 aa, synthetic construct
  REGION 1..109: note = Synthetic
QAVVTQEPSLTVSPGGTVTLTCASSTGAVTTGNYPNWFQQKPGQAFRGLIAGTNNKASWTPARFSGSLLGDKAALTLSGVQPEDEAEYYCALWFSSHWVFGGGTKLTVL

SEQ ID NO: 50 - protein, 238 aa, synthetic construct
  REGION 1..238: note = Synthetic
QAVVTQESALTTSPGETVTLTCRSSTGAVTTGNYPNWVQEKPDHLFTGLIAGTNNRAPGVPARFSGSLIGDKAALTITGAQTEDEAIYFCALWFSSHWVFGGGTKLTVLGGGGSGGGGSGGGGSEVQLQQSGPVLVKPGASVKISCKASGYSFTGYYMHWVRQSLVKRLEWIGRINPYTGVPSYKHNFKDKASLTVDKSSSTAYMELHSLTSEDSAVYYCARELGGYWGQGTTLTVSS

SEQ ID NO: 51 - protein, 239 aa, synthetic construct
  REGION 1..239: note = Synthetic
MEVQLQQSGPVLVKPGASVKISCKASGYSFTGYYMHWVRQSLVKRLEWIGRINPYTGVPSYKHNFKDKASLTVDKSSSTAYMELHSLTSEDSAVYYCARELGGYWGQGTTLTVSSGGGGSGGGGSGGGGSQAVVTQESALTTSPGETVTLTCRSSTGAVTTGNYPNWVQEKPDHLFTGLIAGTNNRAPGVPARFSGSLIGDKAALTITGAQTEDEAIYFCALWFSSHWVFGGGTKLTVL

SEQ ID NO: 52 - protein, 238 aa, synthetic construct
  REGION 1..238: note = Synthetic
QAVVTQESALTTSPGETVTLTCRSSTGAVTTGNYPNWVQEKPDHLFTGLIAGTNNRAPGVPARFSGSLIGDKAALTITGAQTEDEAIYFCALWFSSHWVFGCGTKLTVLGGGGSGGGGSGGGGSEVQLQQSGPVLVKPGASVKISCKASGYSFTGYYMHWVRQSLVKCLEWIGRINPYTGVPSYKHNFKDKASLTVDKSSSTAYMELHSLTSEDSAVYYCARELGGYWGQGTTLTVSS

SEQ ID NO: 53 - protein, 239 aa, synthetic construct
  REGION 1..239: note = Synthetic
MEVQLQQSGPVLVKPGASVKISCKASGYSFTGYYMHWVRQSLVKCLEWIGRINPYTGVPSYKHNFKDKASLTVDKSSSTAYMELHSLTSEDSAVYYCARELGGYWGQGTTLTVSSGGGGSGGGGSGGGGSQAVVTQESALTTSPGETVTLTCRSSTGAVTTGNYPNWVQEKPDHLFTGLIAGTNNRAPGVPARFSGSLIGDKAALTITGAQTEDEAIYFCALWFSSHWVFGCGTKLTVL

SEQ ID NO: 54 - protein, 239 aa, synthetic construct
  REGION 1..239: note = Synthetic
QVQLQQSGPELEKPGASVKISCKASGYSFTGYYMHWVKQSHGKCLEWIGRINPYTGVPSYKHNFKDKATLTVDKSSSTAYMDLLSLTSEDSAVYFCARELGGYWGQGTTVTVSSGGGGSGGGGSGGGGSDIELTQSPAIMSASPGEKVTMTCRSSTGAVTTGNYPNWYQQKSGTSPKGLIAGTNNRAPGVPGRFSGSGSGNSYSLTISSVEAEDDATYYCALWFSSHWVFGCGTKLEIK

SEQ ID NO: 55 - protein, 114 aa, Mus musculus; identical to SEQ ID NO: 44

SEQ ID NO: 56 - protein, 239 aa, synthetic construct
  REGION 1..239: note = Synthetic
DIQMTQSPSSLSASVGDRVTITCRSSTGAVTTGNYPNWYQQKSGKAPKGLIAGTNNRAPGVPSRFSGSGSGTDFTLTISSLQPEDFATYYCALWFSSHWVFGQGTKLEIKGGGGSGGGGSGGGGSQVQLVQSGAEVKKPGASVKVSCKASGYSFTGYYMHWVRQAPGQGLEWIGRINPYTGVPSYKHNFKDKATMTVDTSTSTVYMELSSLRSEDTAVYYCARELGGYWGQGTLVTVSS

SEQ ID NO: 57 - protein, 239 aa, synthetic construct
  REGION 1..239: note = Synthetic
QVQLVQSGAEVKKPGASVKVSCKASGYSFTGYYMHWVRQAPGQGLEWIGRINPYTGVPSYKHNFKDKATMTVDTSTSTVYMELSSLRSEDTAVYYCARELGGYWGQGTLVTVSSGGGGSGGGGSGGGGSDIQMTQSPSSLSASVGDRVTITCRSSTGAVTTGNYPNWYQQKSGKAPKGLIAGTNNRAPGVPSRFSGSGSGTDFTLTISSLQPEDFATYYCALWFSSHWVFGQGTKLEIK

SEQ ID NO: 58 - protein, 238 aa, synthetic construct
  REGION 1..238: note = Synthetic
QAVVTQEPSLTVSPGGTVTLTCASSTGAVTTGNYPNWFQEKPGQAFRGLIAGTNNRAPWVPARFSGSLIGDKAALTLSGVQPEDEAEYFCALWFSSHWVFGGGTKLTVLGGGGSGGGGSGGGGSEVQLVQSGAEVKKPGASVKVSCKASGYSFTGYYMHWVRQAPGQGLEWIGRINPYTGVPSYKHKFQGRVTLTVDKSTSTAYMELSSLRSEDTAVYYCARELGGYWGQGTTVTVSS

SEQ ID NO: 59 - protein, 238 aa, synthetic construct
  REGION 1..238: note = Synthetic
QAVVTQEPSLTVSPGGTVTLTCASSTGAVTTGNYPNWFQEKPGQAFRGLIAGTNNRAPWVPARFSGSLIGDKAALTLSGVQPEDEAEYFCALWFSSHWVFGGGTKLTVLGGGGSGGGGSGGGGSQVQLVQSGAEVKKPGASVKVSCKASGYSFTGYYMHWVRQAPGQGLEWMGRINPYTGVPSYKHKFQGRVTMTVDKSTSTAYMELSSLRSEDTAVYYCARELGGYWGQGTTVTVSS

SEQ ID NO: 60 - protein, 238 aa, synthetic construct
  REGION 1..238: note = Synthetic
QAVVTQEPSLTVSPGGTVTLTCASSTGAVTTGNYPNWFQQKPGQAFRGLIAGTNNKASWTPARFSGSLLGDKAALTLSGVQPEDEAEYYCALWFSSHWVFGGGTKLTVLGGGGSGGGGSGGGGSEVQLVQSGAEVKKPGASVKVSCKASGYSFTGYYMHWVRQAPGQGLEWIGRINPYTGVPSYKHKFQGRVTLTVDKSTSTAYMELSSLRSEDTAVYYCARELGGYWGQGTTVTVSS

SEQ ID NO: 61 - protein, 238 aa, synthetic construct
  REGION 1..238: note = Synthetic
QAVVTQEPSLTVSPGGTVTLTCASSTGAVTTGNYPNWFQQKPGQAFRGLIAGTNNKASWTPARFSGSLLGDKAALTLSGVQPEDEAEYYCALWFSSHWVFGGGTKLTVLGGGGSGGGGSGGGGSQVQLVQSGAEVKKPGASVKVSCKASGYSFTGYYMHWVRQAPGQGLEWMGRINPYTGVPSYKHKFQGRVTMTVDKSTSTAYMELSSLRSEDTAVYYCARELGGYWGQGTTVTVSS

SEQ ID NO: 62 - protein, 238 aa, synthetic construct
  REGION 1..238: note = Synthetic
QTVVTQEPSLTVSPGGTVTLTCRSSTGAVTTGNYPNWFQQKPGQAPRALIAGTNNRAPWTPARFSGSLIGDKAALTLSGVQPEDEAEYYCALWFSSHWVFGGGTKLTVLGGGGSGGGGSGGGGSQVQLVQSGAEVKKPGASVKVSCKASGYSFTGYYMHWVRQAPGQGLEWMGRINPYTGVPSYAQKFQGRVTMTVDKSSSTAYMELSRLRSDDTAVYYCARELGGYWGQGTLVTVSS

SEQ ID NO: 63 - protein, 238 aa, synthetic construct
  REGION 1..238: note = Synthetic
QTVVTQEPSLTVSPGGTVTLTCRSSTGAVTTGNYPNWFQQKPGQAPRALIAGTNNRAPGVPARFSGSLIGDKAALTLSGVQPEDEAEYYCALWFSSHWVFGGGTKLTVLGGGGSGGGGSGGGGSQVQLVQSGAEVKKPGASVKVSCKASGYSFTGYYMHWVRQAPGQGLEWMGRINPYTGVPSYAQKFQGRVTMTVDKSSSTAYMELSRLRSDDTAVYYCARELGGYWGQGTLVTVSS

SEQ ID NO: 64 - protein, 238 aa, synthetic construct
  REGION 1..238: note = Synthetic
QTVITQEPSLTVSPGGTVTLTCRSSTGAVTTGNYPNWFQQKPGQAPRALIAGTNNRAPGVPARFSGSLIGDKAALTLSGVQPEDEAEYYCALWFSSHWVFGGGTKLTVLGGGGSGGGGSGGGGSQVQLVQSGAEVKKPGASVKVSCKASGYSFTGYYMHWVRQAPGQGLEWMGRINPYTGVPSYAQKFQGRVTMTVDKSSSTAYMELSRLRSDDTAVYYCARELGGYWGQGTLVTVSS

SEQ ID NO: 65 - protein, 238 aa, synthetic construct
  REGION 1..238: note = Synthetic
QTVVTQEPSLTVSPGGTVTLTCRSSTGAVTTGNYPNWFQQKPGQAPRAIIAGTNNRAPGVPARFSGSLIGDKAALTISGVQPEDEAEYYCALWFSSHWVFGGGTKLTVLGGGGSGGGGSGGGGSQVQLVQSGAEVKKPGASVKVSCKASGYSFTGYYMHWVRQAPGQGLEWMGRINPYTGVPSYAQKFQGRVTMTVDKSSSTAYMELSRLRSDDTAVYYCARELGGYWGQGTLVTVSS

SEQ ID NO: 66 - protein, 238 aa, synthetic construct
  REGION 1..238: note = Synthetic
QTVVTQEPSLTVSPGGTVTLTCRSSTGAVTTGNYPNWFQQKPGQAPRALIAGTNNRAPGVPARFSGSLIGDKAALTLSGVQPEDEAEYYCALWFSSHWVFGGGTKLTVLGGGGSGGGGSGGGGSQVQLVQSGAEVKKPGASVKVSCKASGYSFTGYYMHWVRQAPGQGLEWMGRINPYTGVPSYAQKFQGRVTLTVDKSSSTAYMELSRLRSDDTAVYYCARELGGYWGQGTLVTVSS

SEQ ID NO: 67 - protein, 238 aa, synthetic construct
  REGION 1..238: note = Synthetic
QTVVTQEPSLTVSPGGTVTLTCRSSTGAVTTGNYPNWFQQKPGQAPRALIAGTNNRAPGVPARFSGSLIGDKAALTLSGVQPEDEAEYYCALWFSSHWVFGGGTKLTVLGGGGSGGGGSGGGGSQVQLVQSGAEVKKPGASVKVSCKASGYSFTGYYMHWVRQVPGQGLEWMGRINPYTGVPSYAQKFQGRVTMTVDKSSSTAYMEISRLRSDDTAVYYCARELGGYWGQGTLVTVSS

SEQ ID NO: 68 - protein, 238 aa, synthetic construct
  REGION 1..238: note = Synthetic
QTVVTQEPSLTVSPGGTVTLTCRSSTGAVTTGNYPNWFQQKPGQAPRAIIAGTNNRAPGVPARFSGSLIGDKAALTISGVQPEDEAEYYCALWFSSHWVFGGGTKLTVLGGGGSGGGGSGGGGSQVQLVQSGAEVKKPGASVKVSCKASGYSFTGYYMHWVRQVPGQGLEWMGRINPYTGVPSYAQKFQGRVTMTVDKSSSTAYMEISRLRSDDTAVYYCARELGGYWGQGTLVTVSS

SEQ ID NO: 69 - protein, 238 aa, synthetic construct
  REGION 1..238: note = Synthetic
QTVITQEPSLTVSPGGTVTLTCRSSTGAVTTGNYPNWFQQKPGQAPRAIIAGTNNRAPGVPARFSGSLIGDKAALTISGVQPEDEAEYYCALWFSSHWVFGGGTKLTVLGGGGSGGGGSGGGGSQVQLVQSGAEVKKPGASVKISCKASGYSFTGYYMHWVRQVPGQGLEWMGRINPYTGVPSYAQKFQGRVTLTVDKSSSTAYMEISRLRSDDTAVYYCARELGGYWGQGTLVTVSS

SEQ ID NO: 70 - protein, 461 aa, synthetic construct
  REGION 1..461: note = Synthetic
MQAVVTQESALTTSPGETVTLTCRSSTGAVTTGNYPNWVQEKPDHLFTGLIAGTNNRAPGVPARFSGSLIGDKAALTITGAQTEDEAIYFCALWFSSHWVFGGGTKLTVLGGGGSGGGGSGGGGSEVQLQQSGPVLVKPGASVKISCKASGYSFTGYYMHWVRQSLVKRLEWIGRINPYTGVPSYKHNFKDKASLTVDKSSSTAYMELHSLTSEDSAVYYCARELGGYWGQGTTLTVSSTSTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 71 - protein, 461 aa, synthetic construct
  REGION 1..461: note = Synthetic
MEVQLQQSGPVLVKPGASVKISCKASGYSFTGYYMHWVRQSLVKRLEWIGRINPYTGVPSYKHNFKDKASLTVDKSSSTAYMELHSLTSEDSAVYYCARELGGYWGQGTTLTVSSGGGGSGGGGSGGGGSQAVVTQESALTTSPGETVTLTCRSSTGAVTTGNYPNWVQEKPDHLFTGLIAGTNNRAPGVPARFSGSLIGDKAALTITGAQTEDEAIYFCALWFSSHWVFGGGTKLTVLTSTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 72 - protein, 461 aa, synthetic construct
  REGION 1..461: note = Synthetic
MQAVVTQEPSLTVSPGGTVTLTCASSTGAVTTGNYPNWFQEKPGQAFRGLIAGTNNRAPWVPARFSGSLIGDKAALTLSGVQPEDEAEYFCALWFSSHWVFGGGTKLTVLGGGGSGGGGSGGGGSEVQLVQSGAEVKKPGASVKVSCKASGYSFTGYYMHWVRQAPGQGLEWIGRINPYTGVPSYKHKFQGRVTLTVDKSTSTAYMELSSLRSEDTAVYYCARELGGYWGQGTTVTVSSTSTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 73 - protein, 461 aa, synthetic construct
  REGION 1..461: note = Synthetic
MQAVVTQEPSLTVSPGGTVTLTCASSTGAVTTGNYPNWFQEKPGQAFRGLIAGTNNRAPWVPARFSGSLIGDKAALTLSGVQPEDEAEYFCALWFSSHWVFGGGTKLTVLGGGGSGGGGSGGGGSQVQLVQSGAEVKKPGASVKVSCKASGYSFTGYYMHWVRQAPGQGLEWMGRINPYTGVPSYKHKFQGRVTMTVDKSTSTAYMELSSLRSEDTAVYYCARELGGYWGQGTTVTVSSTSTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 74 - protein, 461 aa, synthetic construct
  REGION 1..461: note = Synthetic
MQAVVTQEPSLTVSPGGTVTLTCASSTGAVTTGNYPNWFQQKPGQAFRGLIAGTNNKASWTPARFSGSLLGDKAALTLSGVQPEDEAEYYCALWFSSHWVFGGGTKLTVLGGGGSGGGGSGGGGSEVQLVQSGAEVKKPGASVKVSCKASGYSFTGYYMHWVRQAPGQGLEWIGRINPYTGVPSYKHKFQGRVTLTVDKSTSTAYMELSSLRSEDTAVYYCARELGGYWGQGTTVTVSSTSTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 75 - protein, 461 aa, synthetic construct
  REGION 1..461: note = Synthetic
MQAVVTQEPSLTVSPGGTVTLTCASSTGAVTTGNYPNWFQQKPGQAFRGLIAGTNNKASWTPARFSGSLLGDKAALTLSGVQPEDEAEYYCALWFSSHWVFGGGTKLTVLGGGGSGGGGSGGGGSQVQLVQSGAEVKKPGASVKVSCKASGYSFTGYYMHWVRQAPGQGLEWMGRINPYTGVPSYKHKFQGRVTMTVDKSTSTAYMELSSLRSEDTAVYYCARELGGYWGQGTTVTVSSTSTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 76 - protein, 859 aa, synthetic construct
  REGION 1..859: note = Synthetic
MLLLVTSLLLCELPHPAFLLIPHMQAVVTQESALTTSPGETVTLTCRSSTGAVTTGNYPNWVQEKPDHLFTGLIAGTNNRAPGVPARFSGSLIGDKAALTITGAQTEDEAIYFCALWFSSHWVFGGGTKLTVLGGGGSGGGGSGGGGSEVQLQQSGPVLVKPGASVKISCKASGYSFTGYYMHWVRQSLVKRLEWIGRINPYTGVPSYKHNFKDKASLTVDKSSSTAYMELHSLTSEDSAVYYCARELGGYWGQGTTLTVSSTSTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPREGRGSLLTCGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 77 - protein, 859 aa, synthetic construct
  REGION 1..859: note = Synthetic
MLLLVTSLLLCELPHPAFLLIPHMEVQLQQSGPVLVKPGASVKISCKASGYSFTGYYMHWVRQSLVKRLEWIGRINPYTGVPSYKHNFKDKASLTVDKSSSTAYMELHSLTSEDSAVYYCARELGGYWGQGTTLTVSSGGGGSGGGGSGGGGSQAVVTQESALTTSPGETVTLTCRSSTGAVTTGNYPNWVQEKPDHLFTGLIAGTNNRAPGVPARFSGSLIGDKAALTITGAQTEDEAIYFCALWFSSHWVFGGGTKLTVLTSTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPREGRGSLLTCGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 78 - protein, 22 aa, Homo sapiens
MLLLVTSLLLCELPHPAFLLIP

SEQ ID NO: 79 - protein, 238 aa, synthetic construct
  REGION 1..238: note = Synthetic
EVQLQQSGPVLVKPGASVKISCKASGYSFTGYYMHWVRQSLVKRLEWIGRINPYTGVPSYKHNFKDKASLTVDKSSSTAYMELHSLTSEDSAVYYCARELGGYWGQGTTLTVSSGGGGSGGGGSGGGGSQAVVTQESALTTSPGETVTLTCRSSTGAVTTGNYPNWVQEKPDHLFTGLIAGTNNRAPGVPARFSGSLIGDKAALTITGAQTEDEAIYFCALWFSSHWVFGGGTKLTVL

SEQ ID NO: 80 - protein, 45 aa, Homo sapiens
TTTPAPRPPTPAPTIASQPLSLRPEACRPAAGGAVHTRGLDFACD

SEQ ID NO: 81 - protein, 21 aa, Homo sapiens
IYIWAPLAGTCGVLLLSLVIT

SEQ ID NO: 82 - protein, 42 aa, Homo sapiens
KRGRKKLLYIFKQPFMRPVQTTQEEDGCSCRFPEEEEGGCEL

SEQ ID NO: 83 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 84 - protein, 18 aa, synthetic construct
  REGION 1..18: note = Synthetic
EGRGSLLTCGDVEENPGP

SEQ ID NO: 85 - protein, 335 aa, synthetic construct
  REGION 1..335: note = Synthetic
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 86 - protein, 859 aa, synthetic construct
  REGION 1..859: note = Synthetic
MLLLVTSLLLCELPHPAFLLIPHMQAVVTQESALTTSPGETVTLTCRSSTGAVTTGNYPNWVQEKPDHLFTGLIAGTNNRAPGVPARFSGSLIGDKAALTITGAQTEDEAIYFCALWFSSHWVFGGGTKLTVLGGGGSGGGGSGGGGSEVQLQQSGPVLVKPGASVKISCKASGYSFTGYYMHWVRQSLVKRLEWIGRINPYTGVPSYKHNFKDKASLTVDKSSSTAYMELHSLTSEDSAVYYCARELGGYWGQGTTLTVSSTSIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPREGRGSLLTCGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 87 - protein, 859 aa, synthetic construct
  REGION 1..859: note = Synthetic
MLLLVTSLLLCELPHPAFLLIPHMEVQLQQSGPVLVKPGASVKISCKASGYSFTGYYMHWVRQSLVKRLEWIGRINPYTGVPSYKHNFKDKASLTVDKSSSTAYMELHSLTSEDSAVYYCARELGGYWGQGTTLTVSSGGGGSGGGGSGGGGSQAVVTQESALTTSPGETVTLTCRSSTGAVTTGNYPNWVQEKPDHLFTGLIAGTNNRAPGVPARFSGSLIGDKAALTITGAQTEDEAIYFCALWFSSHWVFGGGTKLTVLTSIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPREGRGSLLTCGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 88 - protein, 39 aa, Homo sapiens
IEVMYPPPYLDNEKSNGTIIHVKGKHLCPSPLFPGPSKP

SEQ ID NO: 89 - protein, 27 aa, Homo sapiens
FWVLVVVGGVLACYSLLVTVAFIIFWV

SEQ ID NO: 90 - protein, 832 aa, synthetic construct
  REGION 1..832: note = Synthetic
MLLLVTSLLLCELPHPAFLLIPHMQAVVTQESALTTSPGETVTLTCRSSTGAVTTGNYPNWVQEKPDHLFTGLIAGTNNRAPGVPARFSGSLIGDKAALTITGAQTEDEAIYFCALWFSSHWVFGGGTKLTVLGGGGSGGGGSGGGGSEVQLQQSGPVLVKPGASVKISCKASGYSFTGYYMHWVRQSLVKRLEWIGRINPYTGVPSYKHNFKDKASLTVDKSSSTAYMELHSLTSEDSAVYYCARELGGYWGQGTTLTVSSTSESKYGPPCPPCPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPREGRGSLLTCGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 91 - protein, 832 aa, synthetic construct
  REGION 1..832: note = Synthetic
MLLLVTSLLLCELPHPAFLLIPHMEVQLQQSGPVLVKPGASVKISCKASGYSFTGYYMHWVRQSLVKRLEWIGRINPYTGVPSYKHNFKDKASLTVDKSSSTAYMELHSLTSEDSAVYYCARELGGYWGQGTTLTVSSGGGGSGGGGSGGGGSQAVVTQESALTTSPGETVTLTCRSSTGAVTTGNYPNWVQEKPDHLFTGLIAGTNNRAPGVPARFSGSLIGDKAALTITGAQTEDEAIYFCALWFSSHWVFGGGTKLTVLTSESKYGPPCPPCPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPREGRGSLLTCGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 92 - protein, 12 aa, Homo sapiens
ESKYGPPCPPCP

SEQ ID NO: 93 - protein, 996 aa, synthetic construct
  REGION 1..996: note = Synthetic
MLLLVTSLLLCELPHPAFLLIPHMQAVVTQESALTTSPGETVTLTCRSSTGAVTTGNYPNWVQEKPDHLFTGLIAGTNNRAPGVPARFSGSLIGDKAALTITGAQTEDEAIYFCALWFSSHWVFGGGTKLTVLGGGGSGGGGSGGGGSEVQLQQSGPVLVKPGASVKISCKASGYSFTGYYMHWVRQSLVKRLEWIGRINPYTGVPSYKHNFKDKASLTVDKSSSTAYMELHSLTSEDSAVYYCARELGGYWGQGTTLTVSSTSTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPREGRGSLLTCGDVEENPGPMLLLVTSLLLCELPHPAFLLIPEVQLVESGGGLVQPGGSLRLSCAASGFDLGFYFYACWVRQAPGKGLEWVSCIYTAGSGSTYYASWAKGRFTISRDNSKNTLYLQMNSLRAEDTAVYYCARSTANTRSTYYLNLWGQGTLVTVSSGGGGSGGGGSGGGGSDIQMTQSPSSLSASVGDRVTITCQASQRISSYLSWYQQKPGKVPKLLIYGASTLASGVPSRFSGSGSGTDFTLTISSLQPEDVATYYCQSYAYFDSNNWHAFGGGTKVEIKTSTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 94 - protein, 460 aa, synthetic construct
  REGION 1..460: note = Synthetic
QAVVTQESALTTSPGETVTLTCRSSTGAVTTGNYPNWVQEKPDHLFTGLIAGTNNRAPGVPARFSGSLIGDKAALTITGAQTEDEAIYFCALWFSSHWVFGGGTKLTVLGGGGSGGGGSGGGGSEVQLQQSGPVLVKPGASVKISCKASGYSFTGYYMHWVRQSLVKRLEWIGRINPYTGVPSYKHNFKDKASLTVDKSSSTAYMELHSLTSEDSAVYYCARELGGYWGQGTTLTVSSTSTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 95 - protein, 472 aa, synthetic construct
  REGION 1..472: note = Synthetic
EVQLVESGGGLVQPGGSLRLSCAASGFDLGFYFYACWVRQAPGKGLEWVSCIYTAGSGSTYYASWAKGRFTISRDNSKNTLYLQMNSLRAEDTAVYYCARSTANTRSTYYLNLWGQGTLVTVSSGGGGSGGGGSGGGGSDIQMTQSPSSLSASVGDRVTITCQASQRISSYLSWYQQKPGKVPKLLIYGASTLASGVPSRFSGSGSGTDFTLTISSLQPEDVATYYCQSYAYFDSNNWHAFGGGTKVEIKTSTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 96 - protein, 774 aa, synthetic construct
  REGION 1..774: note = Synthetic
MLLLVTSLLLCELPHPAFLLIPHMEVQLVESGGGLVQPGGSLRLSCAASGFDLGFYFYACWVRQAPGKGLEWVSCIYTAGSGSTYYASWAKGRFTISRDNSKNTLYLQMNSLRAEDTAVYYCARSTANTRSTYYLNLWGQGTLVTVSSGGGGSGGGGSGGGGSDIQMTQSPSSLSASVGDRVTITCQASQRISSYLSWYQQKPGKVPKLLIYGASTLASGVPSRFSGSGSGTDFTLTISSLQPEDVATYYCQSYAYFDSNNWHAFGGGTKVEIKTSTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPREGRGSLLTCGDVEENPGPMLLLVTSLLLCELPHPAFLLIPQAVVTQESALTTSPGETVTLTCRSSTGAVTTGNYPNWVQEKPDHLFTGLIAGTNNRAPGVPARFSGSLIGDKAALTITGAQTEDEAIYFCALWFSSHWVFGGGTKLTVLGGGGSGGGGSGGGGSEVQLQQSGPVLVKPGASVKISCKASGYSFTGYYMHWVRQSLVKRLEWIGRINPYTGVPSYKHNFKDKASLTVDKSSSTAYMELHSLTSEDSAVYYCARELGGYWGQGTTLTVSS

SEQ ID NO: 97 - protein, 513 aa, synthetic construct
MGWSCIILFLVATATGVHSQAVVTQESALTTSPGETVTLTCRSSTGAVTTGNYPNWVQEKPDHLFTGLIAGTNNRAPGVPARFSGSLIGDKAALTITGAQTEDEAIYFCALWFSSHWVFGGGTKLTVLGGGGSGGGGSGGGGSEVQLQQSGPVLVKPGASVKISCKASGYSFTGYYMHWVRQSLVKRLEWIGRINPYTGVPSYKHNFKDKASLTVDKSSSTAYMELHSLTSEDSAVYYCARELGGYWGQGTTLTVSSGGGGSEVQLVESGGGLVQPGRSLRLSCAASGFTFDDYTMHWVRQAPGKGLEWVSGISWNSGSIGYADSVKGRFTISRDNAKKSLYLQMNSLRAEDTALYYCAKDNSGYGHYYYGMDVWGQGTTVTVASGGGGSGGGGSGGGGSEIVMTQSPATLSVSPGERATLSCRASQSVSSNLAWYQQKPGQAPRLLIYGASTRATGIPARFSGSGSGTEFTLTISSLQSEDFAVYYCQHYINWPLTFGGGTKVEIKHHHHHH

SEQ ID NO: 98 - protein, 734 aa, synthetic construct
MGWSCIILFLVATATGVHSQAVVTQESALTTSPGETVTLTCRSSTGAVTTGNYPNWVQEKPDHLFTGLIAGTNNRAPGVPARFSGSLIGDKAALTITGAQTEDEAIYFCALWFSSHWVFGGGTKLTVLGGGGSGGGGSGGGGSEVQLQQSGPVLVKPGASVKISCKASGYSFTGYYMHWVRQSLVKRLEWIGRINPYTGVPSYKHNFKDKASLTVDKSSSTAYMELHSLTSEDSAVYYCARELGGYWGQGTTLTVSSGGGGSEVQLVESGGGLVQPGRSLRLSCAASGFTFDDYTMHWVRQAPGKGLEWVSGISWNSGSIGYADSVKGRFTISRDNAKKSLYLQMNSLRAEDTALYYCAKDNSGYGHYYYGMDVWGQGTTVTVASGGGGSGGGGSGGGGSEIVMTQSPATLSVSPGERATLSCRASQSVSSNLAWYQQKPGQAPRLLIYGASTRATGIPARFSGSGSGTEFTLTISSLQSEDFAVYYCQHYINWPLTFGGGTKVEIKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - protein, 643 aa, synthetic construct
MGWSCIILFLVATATGVHSQAVVTQESALTTSPGETVTLTCRSSTGAVTTGNYPNWVQEKPDHLFTGLIAGTNNRAPGVPARFSGSLIGDKAALTITGAQTEDEAIYFCALWFSSHWVFGGGTKLTVLGGGGSGGGGSGGGGSEVQLQQSGPVLVKPGASVKISCKASGYSFTGYYMHWVRQSLVKRLEWIGRINPYTGVPSYKHNFKDKASLTVDKSSSTAYMELHSLTSEDSAVYYCARELGGYWGQGTTLTVSSGGGGSGGGGSAVQLVESGGGLVQPGNSLRLSCAASGFTFRSFGMSWVRQAPGKEPEWVSSISGSGSDTLYADSVKGRFTISRDNAKTTLYLQMNSLKPEDTAVYYCTIGGSLSRSSQGTQVTVSSGGGGSGGGGSEVQLVESGGGLVQPGRSLRLSCAASGFTFDDYTMHWVRQAPGKGLEWVSGISWNSGSIGYADSVKGRFTISRDNAKKSLYLQMNSLRAEDTALYYCAKDNSGYGHYYYGMDVWGQGTTVTVASGGGGSGGGGSGGGGSEIVMTQSPATLSVSPGERATLSCRASQSVSSNLAWYQQKPGQAPRLLIYGASTRATGIPARFSGSGSGTEFTLTISSLQSEDFAVYYCQHYINWPLTFGGGTKVEIKHHHHHH

SEQ ID NO: 100 - protein, 463 aa, synthetic construct
MGWSCIILFLVATATGVHSEVQLQQSGPVLVKPGASVKISCKASGYSFTGYYMHWVRQSLVKRLEWIGRINPYTGVPSYKHNFKDKASLTVDKSSSTAYMELHSLTSEDSAVYYCARELGGYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 101 - protein, 234 aa, synthetic construct
MGWSCIILFLVATATGVHSQAVVTQESALTTSPGETVTLTCRSSTGAVTTGNYPNWVQEKPDHLFTGLIAGTNNRAPGVPARFSGSLIGDKAALTITGAQTEDEAIYFCALWFSSHWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 102 - protein, 476 aa, synthetic construct
MGWSCIILFLVATATGVHSEVQLVESGGGLVQPGRSLRLSCAASGFTFDDYTMHWVRQAPGKGLEWVSGISWNSGSIGYADSVKGRFTISRDNAKKSLYLQMNSLRAEDTALYYCAKDNSGYGHYYYGMDVWGQGTTVTVASRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 103 - protein, 229 aa, synthetic construct
MGWSCIILFLVATATGVHSEIVMTQSPATLSVSPGERATLSCRASQSVSSNLAWYQQKPGQAPRLLIYGASTRATGIPARFSGSGSGTEFTLTISSLQSEDFAVYYCQHYINWPLTFGGGTKVEIKASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 104 - protein, 468 aa, synthetic construct
MGWSCIILFLVATATGVHSQVQLVQSGAEVKKPGASVKVSCKASGYSFTGYTMNWVRQAPGQGLEWMGLITPYNGASSYNQKFRGKATMTVDTSTSTVYMELSSLRSEDTAVYYCARGGYDGRGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 105 - protein, 232 aa, synthetic construct
MGWSCIILFLVATATGVHSDIQMTQSPSSLSASVGDRVTITCSASSSVSYMHWYQQKSGKAPKLLIYDTSKLASGVPSRFSGSGSGTDFTLTISSLQPEDFATYYCQQWSKHPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 106-107 - skipped (2 entries)

SEQ ID NO: 108 - protein, 473 aa, synthetic construct
MGWSCIILFLVATATGVHSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 109 - protein, 237 aa, synthetic construct
MGWSCIILFLVATATGVHSDIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC